(19) United States
(12) Patent Application Publication
(10) Pub. No.: US 2022/0211832 A1
(43) Pub. Date: Jul. 7, 2022

(54) TREATMENT METHODS

(71) Applicant: Genocea Biosciences, Inc., Cambridge, MA (US)

(72) Inventors: Jessica Baker Flechtner, Sudbury, MA (US); Seth Vollmer Hetherington, Chapel Hill, NC (US); Thomas Charles Heineman, San Diego, CA (US); Lisa K. McNeil, Watertown, MA (US)

(21) Appl. No.: 17/610,643

(22) PCT Filed: May 15, 2020
(86) PCT No.: PCT/US2020/033277

§ 371 Date: Nov. 11, 2021

Related U.S. Application Data
(60) Provisional application No. 62/933,207, filed on Nov. 8, 2019.
(60) Provisional application No. 62/907,262, filed on Sep. 27, 2019.
(60) Provisional application No. 62/855,309, filed on May 31, 2019.
(60) Provisional application No. 62/848,527, filed on May 15, 2019.

(51) Int. Cl. A61K 39/00; G01N 33/50

(52) U.S. Cl. CPC A61K 39/0011; G01N 33/5047; A61K 2039/545; A61K 2039/55; A61K 2039/876; A61K 2039/70; A61K 2039/86; A61K 2039/868; A61K 2039/55561

(57) ABSTRACT

Methods and compositions for identifying tumor antigens of human lymphocytes, and for identifying subjects for cancer therapy, are provided herein. In some embodiments, the method comprises administering to the subject an immunogenic composition comprising one or more selected stimulatory antigens (e.g., one or more stimulatory antigens described herein) or immunogenic fragments thereof, wherein the immunogenic composition is administered according to a dosing regimen comprising an initial dose of the immunogenic composition and additional doses of the immunogenic composition, wherein after an initial dose is administered, an additional dose is administered 3 weeks following the initial dose, an additional dose is administered 6 weeks following the initial dose, an additional dose is administered 12 weeks following the initial dose, and an additional dose is administered 24 weeks following the initial dose.

### CROSS REFERENCE TO RELATED APPLICATIONS

This application claims the benefit of U.S. Provisional Application No. 62/848,527, filed May 15, 2019, U.S. Provisional Application No. 62/855,309, filed May 31, 2019, U.S. Provisional Application No. 62/907,262, filed Sep. 27, 2019, and U.S. Provisional Application No. 62/933,207, filed Nov. 8, 2019, the contents of each of which are hereby incorporated by reference herein in their entirety.

### BACKGROUND

Cancer is characterized by proliferation of abnormal cells. Many treatments include costly and painful surgeries and chemotherapies. Although there is a growing interest in cancer therapies that target cancerous cells using a patient's own immune system, such therapies have had limited success.

### SUMMARY

The present invention features, inter alia, methods of identifying and/or selecting antigens that improve, increase and/or stimulate immune control of a tumor or cancer and methods of administering the same.

Accordingly, one aspect the disclosure features a method of inducing an immune response in a subject. In some embodiments, the method comprises administering to the subject an immunogenic composition comprising one or more selected stimulatory antigens (e.g., one or more stimulatory antigens described herein) or immunogenic fragments thereof, wherein the immunogenic composition is administered according to a dosing regimen comprising an initial dose of the immunogenic composition and additional doses of the immunogenic composition, wherein after an initial dose is administered, an additional dose is administered 3 weeks following the initial dose, an additional dose is administered 6 weeks following the initial dose, an additional dose is administered 12 weeks following the initial dose, and an additional dose is administered 24 weeks following the initial dose.

In some embodiments, the immunogenic composition comprises one or more stimulatory antigens selected by a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject; b) contacting the bacterial cells or beads with antigen presenting cells (APCs) from the subject, wherein the APCs internalize the bacterial cells or beads; c) contacting the APCs with lymphocytes from the subject, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer.

In some embodiments, the method further comprises a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject; b) contacting the bacterial cells or beads with antigen presenting cells (APCs) from the subject, wherein the APCs internalize the bacterial cells or beads; c) contacting the APCs with lymphocytes from the subject, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer.

In some embodiments, the immunogenic composition does not comprise a selected inhibitory antigen (e.g., an inhibitory antigen described herein).

In some embodiments, the immunogenic composition does not comprise an inhibitory antigen selected by a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject; b) contacting the bacterial cells or beads with antigen presenting cells (APCs) from the subject, wherein the APCs internalize the bacterial cells or beads; c) contacting the APCs with lymphocytes from the subject, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting as one or more inhibitory antigens, from among the identified tumor antigens (i) one or more tumor antigens that increase level of expression and/or secretion of one or more immune mediators associated with at least one deleterious and/or non-beneficial response to cancer, and/or (ii) one or more tumor antigens that inhibit and/or suppress level of expression and/or secretion of one or more immune mediators associated with at least one beneficial response to cancer.

In some embodiments, the method further comprises; a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject; b) contacting the bacterial cells or beads with antigen presenting cells (APCs) from the subject, wherein the APCs internalize the bacterial cells or beads; c) contacting the APCs with lymphocytes from the subject, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and f) selecting as one or more inhibitory antigens, from among the identified tumor antigens (i) one or more tumor antigens that increase level of expression and/or secretion of one or more immune mediators associated with at least one deleterious and/or non-beneficial response to cancer, and/or (ii) one or more tumor antigens that inhibit and/or suppress level of expression and/or secretion of one or more immune mediators associated with at least one beneficial response to cancer.

In another aspect, the disclosure features a method of inducing an immune response in a subject. In some embodiments, the method comprises: a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject; b) contacting the bacterial cells or beads with antigen presenting cells (APCs) from the subject, wherein the APCs internalize the bacterial cells or beads; c) contacting the APCs with lymphocytes from the subject, under conditions suitable for activation of lymphocytes by a polypeptide presented by one or more APCs; d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators; e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer; and g) administering to the subject multiple doses of an immunogenic composition comprising one or more of the selected stimulatory antigens, or immunogenic fragments thereof, wherein after an initial dose is administered, a dose is administered 3 weeks following the initial dose, a dose is administered 6 weeks following the initial dose, a dose is administered 12 weeks following the initial dose, and a dose is administered 24 weeks following the initial dose.

In some embodiments, the immunogenic composition does not comprise a selected inhibitory antigen (e.g., an inhibitory antigen described herein). In some embodiments, the one or more of the identified tumor antigens is selected as an inhibitory antigen if (i) the one or more tumor antigens increase level of expression and/or secretion of one or more immune mediators associated with at least one deleterious and/or non-beneficial response to cancer, and/or (ii) the one or more tumor antigens inhibit and/or suppress level of expression and/or secretion of one or more immune mediators associated with at least one beneficial response to cancer.

In some embodiments, the method further comprises selecting as one or more inhibitory antigens, from among the identified tumor antigens (i) one or more tumor antigens that increase level of expression and/or secretion of one or more immune mediators associated with at least one deleterious and/or non-beneficial response to cancer, and/or (ii) one or more tumor antigens that inhibit and/or suppress level of expression and/or secretion of one or more immune mediators associated with at least one beneficial response to cancer.

In some embodiments, the library comprises bacterial cells or beads comprising at least 1, 3, 5, 10, 15, 20, 25, 30, 50, 100, 150, 250, 500, 750, 1000 or more different heterologous polypeptides, or portions thereof.

In some embodiments, the method further comprises determining whether one or more lymphocytes are activated by, or not responsive to, one or more tumor antigens comprises measuring a level of one or more immune mediators.

In some embodiments, the one or more immune mediators are selected from the group consisting of cytokines, soluble mediators, and cell surface markers expressed by the lymphocytes. In some embodiments, the one or more immune mediators are cytokines. In some embodiments, the one or more cytokines are selected from the group consisting of TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANK L), MIP3-alpha, and fractalkine.

In some embodiments, the one or more immune mediators are soluble mediators. In some embodiments, the one or more soluble mediators are selected from the group consisting of granzyme A, granzyme B, sFas, sFasL, perforin, and granulysin.

In some embodiments, the one or more immune mediators are cell surface markers. In some embodiments, the one or more cell surface markers are selected from the group consisting of CD107a, CD107b, CD25, CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), and KLRG1.

In some embodiments, the lymphocytes comprise CD4+ T cells. In some embodiments, the lymphocytes comprise CD8+ T cells. In some embodiments, the lymphocytes comprise NKT cells, gamma-delta T cells, or NK cells. In some embodiments, the lymphocytes comprise any combination of CD4+ T cells, CD8+ T cells, NKT cells, gamma-delta T cells, and NK cells.

In some embodiments, lymphocyte activation is determined by assessing a level of one or more expressed or secreted immune mediators that is at least 20%, 40%, 60%, 80%, 100%, 120%, 140%, 160%, 180%, or 200% higher or lower than a control level.

In some embodiments, lymphocyte activation is determined by assessing a level of one or more expressed or secreted immune mediators that is at least one, two, or three standard deviations greater or lower than the mean of a control level. In some embodiments, the lymphocyte activating is determined by assessing a level of one or more expressed or secreted immune mediators that is at least 1, 2, 3, 4 or 5 median absolute deviations (MADs) greater or lower than a median response level to a control.

In some embodiments, lymphocyte non-responsiveness is determined by assessing a level of one or more expressed or secreted immune mediators that is within 5%, 10%, 15%, or 20% of a control level. In some embodiments, lymphocyte non-responsiveness is determined by assessing a level of one or more expressed or secreted immune mediators that is less than one or two standard deviation higher or lower than the mean of a control level. In some embodiments, lymphocyte non-responsiveness is determined by assessing a level of one or more expressed or secreted immune mediators that is less than one or two median absolute deviation (MAD) higher or lower than a median response level to a control.

In some embodiments, a subject exhibits at least one measure or indication of clinical responsiveness to a cancer therapy. In some embodiments, a subject exhibits at least one measure or indication of failure of clinical responsiveness to a cancer therapy.

In some embodiments, the cancer therapy comprises immune checkpoint blockade therapy. In some embodiments, the immune checkpoint blockade therapy comprises administration of pembrolizumab, nivolumab, ipilimumab, atezolizumab, avelumab, durvalumab, tremelimumab, or cemiplimab. In some embodiments, the immune checkpoint blockade therapy comprises administration of two or more immune checkpoint inhibitors.

In some embodiments, the cancer therapy comprises immune suppression blockade therapy. In some embodiments, the immune suppression blockade therapy comprises administration of Vista (B7-H5, v-domain Ig suppressor of T cell activation) inhibitors, Lag-3 (lymphocyte-activation gene 3, CD223) inhibitors, IDO (indolemamine-pyrrole-2,3,-dioxygenase-1,2) inhibitors, or KIR receptor family (killer cell immunoglobulin-like receptor) inhibitors, CD47 inhibitors, or Tigit (T cell immunoreceptor with Ig and ITIM domain) inhibitors. In some embodiments, the immune suppression blockade therapy comprises administration of two or more immune suppression inhibitors.

In some embodiments, the cancer therapy comprises immune activation therapy. In some embodiments, the immune activation therapy comprises administration of CD40 agonists, GITR (glucocorticoid-induced TNF-R-related protein, CD357) agonists, OX40 (CD134) agonists, 4-1BB (CD137) agonists, ICOS (inducible T cell stimulator, CD278) agonists, IL-2 (interleukin 2) agonists, or interferon agonists. In some embodiments, the immune activation therapy comprises administration of two or more immune activators.

In some embodiments, the cancer therapy comprises adjuvant therapy. In some embodiments, the adjuvant therapy comprises administration of a TLR agonist (e.g., CpG or Poly I:C), STING agonist, non-specific stimulus of innate immunity, dendritic cells, GM-CSF, IL-12, IL-7, Flt-3, or other cytokines.

In some embodiments, the cancer therapy comprises oncolytic virus therapy. In some embodiments, the oncolytic viral therapy comprises administration of talimogene leherparepvec.

In some embodiments, the cancer therapy comprises administration of one or more chemotherapeutic agents. In some embodiments, the cancer therapy comprises radiation. In some embodiments, the cancer therapy comprises surgical excision.

In some embodiments, the cancer therapy comprises cell-based therapy. In some embodiments, the cell-based therapy comprises administration of dendritic cells, chimeric antigen receptor T (CAR-T) cells, T cell receptor-transduced cells, tumor infiltrating lymphocytes (TIL), or natural killer (NK) cells.

In some embodiments, the cancer therapy comprises localized hyperthermia or hypothermia.

In some embodiments, the cancer therapy comprises administration of one or more anti-tumor antibodies. In some embodiments, the anti-tumor antibodies comprise bi-specific antibodies.

In some embodiments, the cancer therapy comprises administration of one or more anti-angiogenic agents. In some embodiments, the cancer therapy comprises any combination of immune checkpoint blockade, immune suppression blockade, immune activation, adjuvant, oncolytic virus, chemotherapeutic, radiation, surgical, cell-based, hyperthermia, hypothermia, anti-tumor antibody, and anti-angiogenic therapies.

In some embodiments, the subject has or is at risk of cancer, and/or exhibits one or more signs or symptoms of cancer, and/or exhibits one or more risk factors for cancer. In some embodiments, the cancer is colorectal cancer, melanoma, bladder cancer, or lung cancer (e.g., non-small cell lung cancer).

In some embodiments, the immune response comprises activation of one or more lymphocytes. In some embodiments, the one or more lymphocytes comprise CD4+ T cells and/or CD8+ T cells and/or NKT cells, gamma-delta T cells, or NK cells. In some embodiments, the one or more lymphocytes comprise any combination of CD4+ T cells, CD8+ T cells, NKT cells, gamma-delta T cells, and NK cells.

In some embodiments, the immune response comprises an increased expression and/or secretion of one or more immune mediators relative to a control. In some embodiments, the one or more immune mediators are cytokines. In some embodiments, the cytokines are selected from TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANK L), MIP3-alpha, MCP1, and fractalkine.

In some embodiments, the immune mediators are soluble mediators. In some embodiments, the one or more soluble mediators are selected from granzyme A, granzyme B, sFas, sFasL, perform, and granulysin.

In some embodiments, the one or more immune mediators are cell surface markers, and the cell surface markers may be selected from CD107a, CD107b, CD25, CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), and KLRG1.

In some embodiments, a level of one or more expressed or secreted immune mediators that is at least 20%, 40%, 60%, 80%, 100%, 120%, 140%, 160%, 180%, or 200% higher than a control level indicates lymphocyte activation. In some embodiments, a level of one or more expressed or secreted immune mediators that is at least one, two, or three standard deviations higher than the mean of a control level indicates lymphocyte activation. In some embodiments, a level of one or more expressed or secreted immune mediators that is at least 1, 2, 3, 4 or 5 median absolute deviations (MADs) higher or lower than a median response level to a control indicates lymphocyte activation.

In some embodiments, the immune response comprises a humoral response and/or a cellular response and the humoral response may comprise an increase in magnitude of response or fold rise from baseline of antigen specific immunoglobulin G (IgG) levels and/or of antigen specific neutralizing antibody levels and/or may comprise a 4-fold or greater rise in IgG titer from baseline and/or may comprise a 2-fold or greater rise in 50% neutralizing antibody titer from baseline.

In some embodiments, the cellular response comprises secretion of granzyme B (GrB) and/or an increase in magnitude of response or fold rise from baseline of granzyme B (GrB) levels and/or an increase in IFN-gamma secretion for T cells.

In some embodiments, the selected stimulatory antigens comprise (i) a tumor antigen described herein (e.g., comprising an amino acid sequence described herein), (ii) a polypeptide having an amino acid sequence at least 90%, 91%, 92%, 93%, 94%, 95%, 96%, 97%, 98%, 99% identical to the amino acid sequence of a tumor antigen described herein, and/or (iii) a polypeptide comprising the amino acid sequence of a tumor antigen described herein having at least one deletion, insertion, and/or translocation. In some embodiments, the method further comprises administering to the subject a cancer therapy or combination of therapies.

### BRIEF DESCRIPTION OF THE DRAWINGS

The present teachings described herein will be more fully understood from the following description of various illustrative embodiments, when read together with the accompanying drawings. It should be understood that the drawings described below are for illustration purposes only and are not intended to limit the scope of the present teachings in any way.

FIG. 1 shows an exemplary dosing regimen represented by Schedule 1.

FIG. 2 shows representative results of in vitro stimulated FluoroSpot assays on CD4+ and CD8+ T cells enriched from PBMCs collected at baseline (prior to vaccination) and at Day 50 from each of 5 patients (patients A, B, C, E, and F).

FIG. 3 shows representative results of ex vivo FluoroSpot assays and in vitro stimulated FluoroSpot assays on CD4+ and CD8+ T cells enriched from PBMCs collected at baseline (prior to vaccination) and at Day 50 from a representative patient (patient E). Panels A and B: ex vivo FluoroSpot assays. Panel C: in vitro stimulated FluoroSpot assays.

FIG. 4 shows representative summary results of ex vivo FluoroSpot assays and in vitro stimulated FluoroSpot assays on total PBMC or PBMCs depleted of CD4+ or CD8+ T cells collected at baseline (prior to vaccination) and at Day 50 from patients A-H and K. Data are reported as the proportion of peptides positive by the DFReq test. Circles represent baseline, squares represent D50 time point. Panel A shows ex vivo FluoroSpot assays for patients A-H, and K. Panel B shows in vitro stimulated FluoroSpot assays for patients A-H, and K. Panel C shows the proportion of SLPs scored positive by any assay for patients A-H, and K.

FIG. 5 shows data for and the status of each patient and includes, for each patient, the tumor type, stage of cancer at diagnosis, period of time from diagnosis, prior therapies the patient received, the patient's calculated tumor mutational burden (TMB), the number of stimulatory and inhibitory neoantigens identified for each patient, and the number of peptides in the example vaccine administered. The graph indicates the status of each patient at different time points within the example vaccination regimen. The timing of example vaccination is indicated by the vertical arrows. The color of the horizontal bars indicates the stage of cancer at diagnosis. A blue horizontal arrow indicates that the patient has not yet completed the vaccination regimen (i.e., is within the dosing period). A black horizontal arrow indicates that the patient has completed the vaccination regimen (i.e., is past the treatment period or post vaccination schedule). A black circle indicates a status of “NED” or no evidence of disease.

FIG. 6 shows representative results of ex vivo dual-analyte FluoroSpot assays on CD4+ and CD8+ T cells enriched from PBMCs of three representative patients (patients A and E; low response patient H). Bulk PBMCs were isolated from the patients at baseline (prior to vaccination) and at the indicated timepoints over the course of their treatment. The secretion of IFNγ and Granzyme B (GrB) was quantified via ex vivo dual-analyte FluoroSpot after stimulation with overlapping peptide pools (OLPs) spanning the patient-specific SLPs used for immunization. In Panel A, data are expressed as mean (±SEM) spot forming cells (SFC) per million PBMCs to each of the four pools. Panel B shows the number of positive pools for each time point. The value above each bar represents the number of subjects contributing data. Grey=prior to vaccination; Blue=post-vaccination. Responses were determined by DFR(eq) test (P<0.05) and SFC greater than the assay LOD.

### DEFINITIONS

Activate: As used herein, a peptide presented by an antigen presenting cell (APC) “activates” a lymphocyte if lymphocyte activity is detectably modulated after exposure to the peptide presented by the APC under conditions that permit antigen-specific recognition to occur. Any indicator of lymphocyte activity can be evaluated to determine whether a lymphocyte is activated, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be systemic or local. In some embodiments, administration may be enteral or parenteral. In some embodiments, administration may be by injection (e.g., intramuscular, intravenous, or subcutaneous injection). In some embodiments, injection may involve bolus injection, drip, perfusion, or infusion. In some embodiments administration may be topical. Those skilled in the art will be aware of appropriate administration routes for use with particular therapies described herein, for example from among those listed on www.fda.gov, which include auricular (otic), buccal, conjunctival, cutaneous, dental, endocervical, endosinusial, endotracheal, enteral, epidural, extra-amniotic, extracorporeal, interstitial, intra-abdominal, intra-amniotic, intra-arterial, intra-articular, intrabiliary, intrabronchial, intrabursal, intracardiac, intracartilaginous, intracaudal, intracavernous, intracavitary, intracerebral, intracisternal, intracorneal, intracoronal, intracorporus cavernosum, intradermal, intranodal, intradiscal, intraductal, intraduodenal, intradural, intraepidermal, intraesophageal, intragastic, intragingival, intralesional, intraluminal, intralymphatic, intramedullary, intrameningeal, intramuscular, intraocular, intraovarian, intrapericardial, intraperitoneal, intrapleural, intraprostatic, intrapulmonary, intrasinal, intraspinal, intrasynovial, intratendinous, intratesticular, intrathecal, intrathoracic, intratubular, intratumor, intratympanic, intrauterine, intravascular, intravenous, intravenous bolus, intravenous drip, intraventricular, intravitreal, laryngeal, nasal, nasogastric, ophthalmic, oral, oropharyngeal, parenteral, percutaneous, periarticular, peridural, perineural, periodontal, rectal, respiratory (e.g., inhalation), retrobulbar, soft tissue, subarachnoid, subconjunctival, subcutaneous, sublingual, submucosal, topical, transdermal, transmucosal, transplacental, transtracheal, ureteral, urethral, or vaginal. In some embodiments, administration may involve electro-osmosis, hemodialysis, infiltration, iontophoresis, irrigation, and/or occlusive dressing. In some embodiments, administration may involve dosing that is intermittent (e.g., a plurality of doses separated in time) and/or periodic (e.g., individual doses separated by a common period of time) dosing. In some embodiments, administration may involve continuous dosing.

Antigen: The term “antigen”, as used herein, refers to a molecule (e.g., a polypeptide) that elicits a specific immune response. Antigen-specific immunological responses, also known as adaptive immune responses, are mediated by lymphocytes (e.g., T cells, B cells, NK cells) that express antigen receptors (e.g., T cell receptors, B cell receptors). In certain embodiments, an antigen is a T cell antigen, and elicits a cellular immune response. In certain embodiments, an antigen is a B cell antigen, and elicits a humoral (i.e., antibody) response. In certain embodiments, an antigen is both a T cell antigen and a B cell antigen. As used herein, the term “antigen” encompasses both a full-length polypeptide as well as a portion or immunogenic fragment of the polypeptide, and a peptide epitope within the polypeptides (e.g., a peptide epitope bound by a Major Histocompatibility Complex (MHC) molecule (e.g., MHC class I, or MHC class II)).

Antigen presenting cell: An “antigen presenting cell” or “APC” refers to a cell that presents peptides on MHC class I and/or MHC class II molecules for recognition by T cells. APC include both professional APC (e.g., dendritic cells, macrophages, B cells), which have the ability to stimulate naïve lymphocytes, and non-professional APC (e.g., fibroblasts, epithelial cells, endothelial cells, glial cells). In certain embodiments, APC are able to internalize (e.g., endocytose) members of a library (e.g., cells of a library of bacterial cells) that express heterologous polypeptides as candidate antigens.

Autolysin polypeptide: An “autolysin polypeptide” is a polypeptide that facilitates or mediates autolysis of a cell (e.g., a bacterial cell) that has been internalized by a eukaryotic cell. In some embodiments, an autolysin polypeptide is a bacterial autolysin polypeptide. Autolysin polypeptides include, and are not limited to, polypeptides whose sequences are disclosed in GenBank® under Acc. Nos. NP_388823.1, NP_266427.1, and P0AGC3.1.

Cancer: As used herein, the term “cancer” refers to a disease, disorder, or condition in which cells exhibit relatively abnormal, uncontrolled, and/or autonomous growth, so that they display an abnormally elevated proliferation rate and/or aberrant growth phenotype characterized by a significant loss of control of cell proliferation. In some embodiments, a cancer may be characterized by one or more tumors. Those skilled in the art are aware of a variety of types of cancer including, for example, adrenocortical carcinoma, astrocytoma, basal cell carcinoma, carcinoid, cardiac, cholangiocarcinoma, chordoma, chronic myeloproliferative neoplasms, craniopharyngioma, ductal carcinoma in situ, ependymoma, intraocular melanoma, gastrointestinal carcinoid tumor, gastrointestinal stromal tumor (GIST), gestational trophoblastic disease, glioma, histiocytosis, leukemia (e.g., acute lymphoblastic leukemia (ALL), acute myeloid leukemia (AML), chronic lymphocytic leukemia (CLL), chronic myelogenous leukemia (CML), hairy cell leukemia, myelogenous leukemia, myeloid leukemia), lymphoma (e.g., Burkitt lymphoma [non-Hodgkin lymphoma], cutaneous T cell lymphoma, Hodgkin lymphoma, mycosis fungoides, Sezary syndrome, AIDS-related lymphoma, follicular lymphoma, diffuse large B-cell lymphoma), melanoma, merkel cell carcinoma, mesothelioma, myeloma (e.g., multiple myeloma), myelodysplastic syndrome, papillomatosis, paraganglioma, pheochromacytoma, pleuropulmonary blastoma, retinoblastoma, sarcoma (e.g., Ewing sarcoma, Kaposi sarcoma, osteosarcoma, rhabdomyosarcoma, uterine sarcoma, vascular sarcoma), Wilms' tumor, and/or cancer of the adrenal cortex, anus, appendix, bile duct, bladder, bone, brain, breast, bronchus, central nervous system, cervix, colon, endometrium, esophagus, eye, fallopian tube, gall bladder, gastrointestinal tract, germ cell, head and neck, heart, intestine, kidney (e.g., Wilms' tumor), larynx, liver, lung (e.g., non-small cell lung cancer, small cell lung cancer), mouth, nasal cavity, oral cavity, ovary, pancreas, rectum, skin, stomach, testes, throat, thyroid, penis, pharynx, peritoneum, pituitary, prostate, rectum, salivary gland, ureter, urethra, uterus, vagina, or vulva.

Cytolysin polypeptide: A “cytolysin polypeptide” is a polypeptide that has the ability to form pores in a membrane of a eukaryotic cell. A cytolysin polypeptide, when expressed in host cell (e.g., a bacterial cell) that has been internalized by a eukaryotic cell, facilitates release of host cell components (e.g., host cell macromolecules, such as host cell polypeptides) into the cytosol of the internalizing cell. In some embodiments, a cytolysin polypeptide is bacterial cytolysin polypeptide. In some embodiments, a cytolysin polypeptide is a cytoplasmic cytolysin polypeptide. Cytolysin polypeptides include, and are not limited to, polypeptides whose sequences are disclosed in U.S. Pat. No. 6,004,815, and in GenBank® under Acc. Nos. NP_463733.1, NP 979614, NP 834769, YP_084586, YP 895748, YP_694620, YP_012823, NP 346351, YP_597752, BAB41212.2, NP_561079.1, YP_001198769, and NP_359331.1.

Cytoplasmic cytolysin polypeptide: A “cytoplasmic cytolysin polypeptide” is a cytolysin polypeptide that has the ability to form pores in a membrane of a eukaryotic cell, and that is expressed as a cytoplasmic polypeptide in a bacterial cell. A cytoplasmic cytolysin polypeptide is not significantly secreted by a bacterial cell. Cytoplasmic cytolysin polypeptides can be provided by a variety of means. In some embodiments, a cytoplasmic cytolysin polypeptide is provided as a nucleic acid encoding the cytoplasmic ccytolysin polypeptide. In some embodiments, a cytoplasmic cytolysin polypeptide is provided attached to a bead. In some embodiments, a cytoplasmic cytolysin polypeptide has a sequence that is altered relative to the sequence of a secreted cytolysin polypeptide (e.g., altered by deletion or alteration of a signal sequence to render it nonfunctional). In some embodiments, a cytoplasmic cytolysin polypeptide is cytoplasmic because it is expressed in a secretion-incompetent cell. In some embodiments, a cytoplasmic cytolysin polypeptide is cytoplasmic because it is expressed in a cell that does not recognize and mediate secretion of a signal sequence linked to the cytolysin polypeptide. In some embodiments, a cytoplasmic cytolysin polypeptide is a bacterial cytolysin polypeptide.

Heterologous: The term “heterologous”, as used herein to refer to genes or polypeptides, refers to a gene or polypeptide that does not naturally occur in the organism in which it is present and/or being expressed, and/or that has been introduced into the organism by the hand of man. In some embodiments, a heterologous polypeptide is a tumor antigen described herein.

Immune mediator: As used herein, the term “immune mediator” refers to any molecule that affects the cells and processes involved in immune responses. Immune mediators include cytokines, chemokines, soluble proteins, and cell surface markers.

Improve, increase, inhibit, stimulate, suppress, or reduce: As used herein, the terms “improve”, “increase”, “inhibit”, “stimulate”, “suppress”, “reduce”, or grammatical equivalents thereof, indicate values that are relative to a baseline or other reference measurement. In some embodiments, an appropriate reference measurement may be or comprise a measurement in a particular system (e.g., in a single individual) under otherwise comparable conditions absent presence of (e.g., prior to and/or after) a particular agent or treatment, or in presence of an appropriate comparable reference agent. The effect of a particular agent or treatment may be direct or indirect. In some embodiments, an appropriate reference measurement may be or may comprise a measurement in a comparable system known or expected to respond in a particular way, in presence of the relevant agent or treatment. In some embodiments, a peptide presented by an antigen presenting cell (APC) “stimulates” or is “stimulatory” to a lymphocyte if the lymphocyte is activated to a phenotype associated with beneficial responses, after exposure to the peptide presented by the APC under conditions that permit antigen-specific recognition to occur, as observed by, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers, relative to a control. In some embodiments, a peptide presented by an antigen presenting cell “suppresses”, “inhibits” or is “inhibitory” to a lymphocyte if the lymphocyte is activated to a phenotype associated with deleterious or non-beneficial responses, after exposure to the peptide presented by the APC under conditions that permit antigen-specific recognition to occur, as observed by, e.g., phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers, relative to a control.

Inhibitory Antigen: An “inhibitory antigen” is an antigen that inhibits, suppresses, impairs and/or reduces immune control of a tumor or cancer. In some embodiments, an inhibitory antigen promotes tumor growth, enables tumor growth, increases and/or enables tumor metastasis, and/or accelerates tumor growth. In some embodiments, an inhibitory antigen stimulates one or more lymphocyte responses that are deleterious or non-beneficial to a subject; and/or inhibits and/or suppresses one or more lymphocyte responses that are beneficial to a subject. In some embodiments, an inhibitory antigen is the target of one or more lymphocyte responses that are deleterious or non-beneficial to a subject; and/or inhibits and/or suppresses one or more lymphocyte responses that are beneficial to a subject.

Invasin polypeptide: An “invasin polypeptide” is a polypeptide that facilitates or mediates uptake of a cell (e.g., a bacterial cell) by a eukaryotic cell. Expression of an invasin polypeptide in a noninvasive bacterial cell confers on the cell the ability to enter a eukaryotic cell. In some embodiments, an invasin polypeptide is a bacterial invasin polypeptide. In some embodiments, an invasin polypeptide is a Yersinia invasin polypeptide (e.g., a Yersinia invasin polypeptide comprising a sequence disclosed in GenBank® under Acc. No. YP_070195.1).

Listeriolysin O (LLO): The terms “listeriolysin O” or “LLO” refer to a listeriolysin O polypeptide of Listeria monocytogenes and truncated forms thereof that retain pore-forming ability (e.g., cytoplasmic forms of LLO, including truncated forms lacking a signal sequence). In some embodiments, an LLO is a cytoplasmic LLO. Exemplary LLO sequences are shown in Table 1, below.

Polypeptide: The term “polypeptide”, as used herein, generally has its art-recognized meaning of a polymer of at least three amino acids. Those of ordinary skill in the art will appreciate, however, that the term “polypeptide” is intended to be sufficiently general as to encompass not only polypeptides having the complete sequence recited herein (or in a reference or database specifically mentioned herein), but also to encompass polypeptides that represent functional fragments (i.e., fragments retaining at least one activity) and immunogenic fragments of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides.

Primary cells: As used herein, “primary cells” refers to cells from an organism that have not been immortalized in vitro. In some embodiments, primary cells are cells taken directly from a subject (e.g., a human). In some embodiments, primary cells are progeny of cells taken from a subject (e.g., cells that have been passaged in vitro). Primary cells include cells that have been stimulated to proliferate in culture.

Response: As used herein, in the context of a subject (a patient or experimental organism), “response”, “responsive”, or “responsiveness” refers to an alteration in a subject's condition that occurs as a result of, or correlates with, treatment. In certain embodiments, a response is a beneficial response. In certain embodiments, a beneficial response can include stabilization of a subject's condition (e.g., prevention or delay of deterioration expected or typically observed to occur absent the treatment), amelioration (e.g., reduction in frequency and/or intensity) of one or more symptoms of the condition, and/or improvement in the prospects for cure of the condition, etc. In certain embodiments, for a subject who has cancer, a beneficial response can include: the subject has a positive clinical response to cancer therapy or a combination of therapies; the subject has a spontaneous response to a cancer; the subject is in partial or complete remission from cancer; the subject has cleared a cancer; the subject has not had a relapse, recurrence or metastasis of a cancer; the subject has a positive cancer prognosis; the subject has not experienced toxic responses or side effects to a cancer therapy or combination of therapies. In certain embodiments, for a subject who had cancer, the beneficial responses occurred in the past, or are ongoing.

In certain embodiments, a response is a deleterious or non-beneficial response. In certain embodiments, a deleterious or non-beneficial response can include deterioration of a subject's condition, lack of amelioration (e.g., no reduction in frequency and/or intensity) of one or more symptoms of the condition, and/or degradation in the prospects for cure of the condition, etc. In certain embodiments, for a subject who has cancer, a deleterious or non-beneficial response can include: the subject has a negative clinical response to cancer therapy or a combination of therapies; the subject is not in remission from cancer; the subject has not cleared a cancer; the subject has had a relapse, recurrence or metastasis of a cancer; the subject has a negative cancer prognosis; the subject has experienced toxic responses or side effects to a cancer therapy or combination of therapies. In certain embodiments, for a subject who had cancer, the deleterious or non-beneficial responses occurred in the past, or are ongoing.

As used herein, in the context of a cell, organ, tissue, or cell component, e.g., a lymphocyte, “response”, “responsive”, or “responsiveness” refers to an alteration in cellular activity that occurs as a result of, or correlates with, administration of or exposure to an agent, e.g. a tumor antigen. In certain embodiments, a beneficial response can include increased expression and/or secretion of immune mediators associated with positive clinical responses or outcomes in a subject. In certain embodiments, a beneficial response can include decreased expression and/or secretion of immune mediators associated with negative clinical response or outcomes in a subject. In certain embodiments, a deleterious or non-beneficial response can include increased expression and/or secretion of immune mediators associated with negative clinical responses or outcomes in a subject. In certain embodiments, a deleterious or non-beneficial response can include decreased expression and/or secretion of immune mediators associated with positive clinical responses or outcomes in a subject. In certain embodiments, a response is a clinical response. In certain embodiments, a response is a cellular response. In certain embodiments, a response is a direct response. In certain embodiments, a response is an indirect response. In certain embodiments, “non-response”, “non-responsive”, or “non-responsiveness” mean minimal response or no detectable response. In certain embodiments, a “minimal response” includes no detectable response. In certain embodiments, presence, extent, and/or nature of response can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray, CT scan, MRI, ultrasound, endoscopy, laparoscopy, presence or level of a particular marker in a sample, cytology, and/or histology. Where a response of interest is a response of a tumor to a therapy, ones skilled in the art will be aware of a variety of established techniques for assessing such response, including, for example, for determining tumor burden, tumor size, tumor stage, etc. Methods and guidelines for assessing response to treatment are discussed in Therasse et al., J. Natl. Cancer Inst., 2000, 92(3):205-216; and Seymour et al., Lancet Oncol., 2017, 18:e143-52. The exact response criteria can be selected in any appropriate manner, provided that when comparing groups of tumors, patients or experimental organism, and/or cells, organs, tissues, or cell components, the groups to be compared are assessed based on the same or comparable criteria for determining response rate. One of ordinary skill in the art will be able to select appropriate criteria.

Stimulatory Antigen: A “stimulatory antigen” is an antigen that improves, increases and/or stimulates immune control of a tumor or cancer. In some embodiments, a stimulatory antigen is the target of an immune response that reduces, kills, shrinks, resorbs, and/or eradicates tumor growth; does not enable tumor growth; decreases tumor metastasis, and/or decelerates tumor growth. In some embodiments, a stimulatory antigen inhibits and/or suppresses one or more lymphocyte responses that are deleterious or non-beneficial to a subject; and/or stimulates one or more lymphocyte responses that are beneficial to a subject.

Tumor: As used herein, the term “tumor” refers to an abnormal growth of cells or tissue. In some embodiments, a tumor may comprise cells that are precancerous (e.g., benign), malignant, pre-metastatic, metastatic, and/or non-metastatic. In some embodiments, a tumor is associated with, or is a manifestation of, a cancer. In some embodiments, a tumor may be a disperse tumor or a liquid tumor. In some embodiments, a tumor may be a solid tumor.

### DETAILED DESCRIPTION

Recent advances in immune checkpoint inhibitor therapies such as ipilimumab, nivolumab, and pembrolizumab for cancer immunotherapy have resulted in dramatic efficacy in subjects suffering from NSCLC, among other indications. Nivolumab and pembroluzimab have been approved by the Food and Drug Administration (FDA) and European Medicines Agency (EMA) for use in patients with advanced NSCLC who have previously been treated with chemotherapy. They have solidified the importance of T cell responses in control of tumors. Neoantigens, potential cancer rejection antigens that are entirely absent from the normal human genome, are postulated to be relevant to tumor control; however, attempts to define them and their role in tumor clearance has been hindered by the paucity of available tools to define them in a biologically relevant and unbiased way (Schumacher and Schreiber, 2015 Science 348:69-74, Gilchuk et al., 2015 Curr Opin Immunol 34:43-51)

Taking non-small cell lung carcinoma (NSCLC) as an example, whole exome sequencing of NSCLC tumors from patients treated with pembrolizumab showed that higher non-synonymous mutation burden in tumors was associated with improved objective response, durable clinical benefit, and progression-free survival (Rizvi et al., (2015) Science 348(6230): 124-8). In this study, the median non-synonymous mutational burden of the discovery cohort was 209 and of the validation cohort was 200. However, simply because a mutation was identified by sequencing, does not mean that the epitope it creates can be recognized by a T cell or serves as a protective antigen for T cell responses (Gilchuk et al., 2015 Curr Opin Immunol 34:43-51), making the use of the word neoantigen somewhat of a misnomer. With 200 or more potential targets of T cells in NSCLC, it is not feasible to test every predicted epitope to determine which of the mutations serve as neoantigens, and which neoantigens are associated with clinical evidence of tumor control. Recently, a study by McGranahan et al., showed that clonal neoantigen burden and overall survival in primary lung adenocarcinomas are related. However, even enriching for clonal neoantigens results in potential antigen targets ranging from 50 to approximately 400 (McGranahan et al., 2016 Science 351:1463-69). Similar findings have been described for melanoma patients who have responded to ipilimumab therapy (Snyder et al., 2015 NEJM; Van Allen et al., 2015 Science) and in patients with mismatch-repair deficient colorectal cancer who were treated with pembrolizumab (Le et al., 2015 NEJM).

In well-established tumors, activation of endogenous anti-tumor T cell responses is often insufficient to result in complete tumor regression. Moreover, T cells that have been educated in the context of the tumor microenvironment sometimes are sub-optimally activated, have low avidity, and ultimately fail to recognize the tumor cells that express antigen. In addition, tumors are complex and comprise numerous cell types with varying degrees of expression of mutated genes, making it difficult to generate polyclonal T cell responses that are adequate to control tumor growth. As a result, researchers in the field have proposed that it is important in cancer subjects to identify the mutations that are “potential tumor antigens” in addition to those that are confirmed in the cancer subject to be recognized by their T cells.

There are currently no reliable methods of identifying potential tumor antigens in a comprehensive way. Computational methods have been developed in an attempt to predict what is an antigen, however there are many limitations to these approaches. First, modeling epitope prediction and presentation needs to take into account the greater than 12,000 HLA alleles encoding MHC molecules, with each subject expressing as many as 14 of them, all with different epitope affinities. Second, the vast majority of predicted epitopes fail to be found presented by tumors when they are evaluated using mass spectrometry. Third, the predictive algorithms do not take into account T cell recognition of the antigen, and the majority of predicted epitopes are incapable of eliciting T cell responses even when they are present. Finally, the second arm of cellular immunity, the CD4+ T cell subset, is often overlooked; the majority of in silico tools focus on MHC class I binders. The tools for predicting MHC class II epitopes are under-developed and more variable.

Cancer immune therapies boost immune responses, mainly T cell responses, to kill cancer cells while sparing normal cells. The success of checkpoint blockade immunotherapies in producing durable remission in a significant subset of cancer patients has reinforced that immunotherapeutic interventions can result in tumor control. Additionally, they have demonstrated the importance of tumor reactive T cells in antitumor efficacy. Despite significant progress, however, checkpoint inhibitor therapy is effective in only 20%-30% of treated patients. Therefore, there remains a large unmet need for safe and effective immune therapies that might be applicable to a broader range of tumor types.

A hallmark of tumorigenesis is the accumulation of mutations in cancer cells. These mutations are found as both driver and passenger events and they are exclusively present in tumor but not in normal tissue. The mutated protein fragments that are presented by the peptide human leukocyte antigen (pHLA) complexes on the cell surface and recognizable by the immune system are known as neoantigens. Neoantigens may induce reactive T cells that can mediate the killing of cancer cells by the host immune system (1, 2). There is a substantial body of evidence supporting a critical role for neoantigens in anti tumor control by marking the cancer cells as non-self, which leads to immune system targeting for destruction:
Neoantigens represent dominant targets in tumor-infiltrating lymphocyte populations in patients benefiting from adoptive T cell therapy, and a neoantigen specific T cell population was sufficient to induce tumor regression in mouse and man (3, 4).
The widespread detection of spontaneously occurring neoantigen-specific T cells demonstrates that processing and presentation of multiple neoantigens on tumors occurs despite the current insensitivity of biochemical detection (5-7)
Checkpoint blockade therapy has revealed new and amplified neoantigen-specific T cell responses which, in the mouse, are central to disease control (7, 8).
A retrospective meta-analysis of 6 tumor types showed that overall survival was improved in patients predicted to have at least 1 immunogenic neoantigen epitope (9).
Memory cytotoxic T lymphocyte responses to mutated antigens are generated in patients with unexpected long-term survival or those who have undergone effective immunotherapy (10, 11).
A neoantigen-specific CD4 T cell product caused regression of a metastatic cholangiocarcinoma (12).

Because of the tumor exclusivity of neoantigens, they can serve as tumor-specific targets for T cell-mediated recognition and destruction of tumor cells. Cancer vaccines targeting neoantigens are expected to be effective in activating T cells that can recognize and kill tumors. Several clinical trials have been initiated to directly test neoantigen vaccines. Most solid tumors harbor over 100 non-synonymous mutations; however, not all mutated proteins are processed and presented to T cells by pHLA. So far, most neoantigen vaccines depend on algorithms to identify which mutations detected in tumors are the appropriate neoantigens for inclusion in the vaccine. The present disclosure provides methods and systems for the rapid identification of tumor antigens (e.g., tumor specific antigens (TSAs, or neoantigens), tumor associated antigens (TAAs), or cancer/testis antigens (CTAs)) that elicit T cell responses and particularly that elicit human T cell responses, as well as polypeptides that are potential tumor antigens. For purposes of this disclosure, “tumor antigens” includes both tumor antigens and potential tumor antigens. As described herein, methods of the present disclosure identified stimulatory tumor antigens that were not identified by known algorithms. Further, methods of the present disclosure identified suppressive and/or inhibitory tumor antigens that are not identifiable by known algorithms. Methods of the present disclosure also identified polypeptides that are potential tumor antigens, i.e., polypeptides that activate T cells of non-cancerous subjects, but not T cells of subjects suffering from cancer. The present disclosure also provides methods of selecting tumor antigens and potential tumor antigens, methods of using the selected tumor antigens and potential tumor antigens, immunogenic compositions comprising the selected tumor antigens and potential tumor antigens, and methods of manufacturing immunogenic compositions.

The present disclosure further provides methods for identifying stimulatory and/or inhibitory antigens in a particular subject suffering from cancer. Generally, potential tumor antigens may be identified from a tumor sample from the subject; a library of bacterial cells or beads comprising a plurality of tumor antigens may be generated, where each bacterial cell or bead of the library comprises a different tumor antigen; APCs from the patient can then be contacted with and internalize the bacterial cells or beads. The subject's T cells are then exposed to APCs expressing the potential antigens. Stimulatory and inhibitory antigens may then be identified based on the measuring T cell response to the different antigens.

### Library Generation

A library is a collection of members (e.g., cells or non-cellular particles, such as virus particles, liposomes, or beads (e.g., beads coated with polypeptides, such as in vitro translated polypeptides, e.g., affinity beads, e.g., antibody coated beads, or NTA-Ni beads bound to polypeptides of interest). According to the present disclosure, members of a library include (e.g., internally express or carry) polypeptides of interest described herein. In some embodiments, members of a library are cells that internally express polypeptides of interest described herein. In some embodiments, members of a library which are particles carry, and/or are bound to, polypeptides of interest. Use of a library in an assay system allows simultaneous evaluation in vitro of cellular responses to multiple candidate antigens. According to the present disclosure, a library is designed to be internalized by human antigen presenting cells so that peptides from library members, including peptides from internally expressed polypeptides of interest, are presented on MHC molecules of the antigen presenting cells for recognition by T cells.

Libraries can be used in assays that detect peptides presented by human MHC class I and MHC class II molecules. Polypeptides expressed by the internalized library members are digested in intracellular endocytic compartments (e.g., phagosomes, endosomes, lysosomes) of the human cells and presented on MHC class II molecules, which are recognized by human CD4⁺ T cells. In some embodiments, library members include a cytolysin polypeptide, in addition to a polypeptide of interest. In some embodiments, library members include an invasin polypeptide, in addition to the polypeptide of interest. In some embodiments, library members include an autolysin polypeptide, in addition to the polypeptide of interest. In some embodiments, library members are provided with cells that express a cytolysin polypeptide (i.e., the cytolysin and polypeptide of interest are not expressed in the same cell, and an antigen presenting cell is exposed to members that include the cytolysin and members that include the polypeptide of interest, such that the antigen presenting cell internalizes both, and such that the cytolysin facilitates delivery of polypeptides of interest to the MHC class I pathway of the antigen presenting cell). A cytolysin polypeptide can be constitutively expressed in a cell, or it can be under the control of an inducible expression system (e.g., an inducible promoter). In some embodiments, a cytolysin is expressed under the control of an inducible promoter to minimize cytotoxicity to the cell that expresses the cytolysin.

Once internalized by a human cell, a cytolysin polypeptide perforates intracellular compartments in the human cell, allowing polypeptides expressed by the library members to gain access to the cytosol of the human cell. Polypeptides released into the cytosol are presented on MHC class I molecules, which are recognized by CD8⁺ T cells.

A library can include any type of cell or particle that can be internalized by and deliver a polypeptide of interest (and a cytolysin polypeptide, in applications where a cytolysin polypeptide is desirable) to, antigen presenting cells for use in methods described herein. Although the term “cell” is used throughout the present specification to refer to a library member, it is understood that, in some embodiments, the library member is a non-cellular particle, such as a virus particle, liposome, or bead. In some embodiments, members of the library include polynucleotides that encode the polypeptide of interest (and cytolysin polypeptide), and can be induced to express the polypeptide of interest (and cytolysin polypeptide) prior to, and/or during internalization by antigen presenting cells.

In some embodiments, the cytolysin polypeptide is heterologous to the library cell in which it is expressed, and facilitates delivery of polypeptides expressed by the library cell into the cytosol of a human cell that has internalized the library cell. Cytolysin polypeptides include bacterial cytolysin polypeptides, such as listeriolysin O (LLO), streptolysin O (SLO), and perfringolysin O (PFO). Additional cytolysin polypeptides are described in U.S. Pat. No. 6,004,815. In certain embodiments, library members express LLO. In some embodiments, a cytolysin polypeptide is not significantly secreted by the library cell (e.g., less than 20%, 10%, 5%, or 1% of the cytolysin polypeptide produced by the cell is secreted). For example, the cytolysin polypeptide is a cytoplasmic cytolysin polypeptide, such as a cytoplasmic LLO polypeptide (e.g., a form of LLO which lacks the N-terminal signal sequence, as described in Higgins et al., Mol. Microbiol. 31(6):1631-1641,1999). Exemplary cytolysin polypeptide sequences are shown in Table 1. The listeriolysin O (Δ3-25) sequence shown in the second row of Table 1 has a deletion of residues 3-25, relative to the LLO sequence in shown in the first row of Table 1, and is a cytoplasmic LLO polypeptide. In some embodiments, a cytolysin is expressed constitutively in a library host cell. In other embodiments, a cytolysin is expressed under the control of an inducible promoter. Cytolysin polypeptides can be expressed from the same vector, or from a different vector, as the polypeptide of interest in a library cell.

TABLE 1
Exemplary Cytolysin Polypeptides

| | Polypeptide | |
|---|---|---|
| Polypeptide Name | Accession No. | |
| (species) | GI No. | Polypeptide Sequence |
| listeriolysin O | NP_463733.1 | MKKIMLVFITLILVSLPIAQQTEAKDASAFNKENSISSMAPPASP |
| (Listeria | GI: 16802248 | PASPKTPIEKKHADEIDKYIQGLDYNKNNVLVYHGDAVTNVPPRK |
| monocytogenes) | | GYKDGNEYIVVEKKKKSINQNNADIQVVNAISSLTYPGALVKANS |
| | | ELVENQPDVLPVKRDSLTLSIDLPGMTNQDNKIVVKNATKSNVNN |
| | | AVNTLVERWNEKYAQAYPNVSAKIDYDDEMAYSESQLIAKFGTAF |
| | | KAVNNSLNVNFGAISEGKMQEEVISFKQIYYNVNVNEPTRPSRFF |
| | | GKAVTKEQLQALGVNAENPPAYISSVAYGRQVYLKLSTNSHSTKV |
| | | KAAFDAAVSGKSVSGDVELTNIIKNSSFKAVIYGGSAKDEVQIID |
| | | GNLGDLRDILKKGATFNRETPGVPIAYTTNFLKDNELAVIKNNSE |
| | | YIETTSKAYTDGKINIDHSGGYVAQFNISWDEVNYDPEGNEIVQH |
| | | KNWSENNKSKLAHFTSSIYLPGNARNINVYAKECTGLAWEWWRTV |
| | | IDDRNLPLVKNRNISIWGTTLYPKYSNKVDNPIE |
| | | (SEQ ID NO: 1) |
| listeriolysin O | | MKDASAFNKENSISSMAPPASPPASPKTPIEKKHADEIDKYIQGL |
| (43-25) | | DYNKNNVLVYHGDAVTNVPPRKGYKDGNEYIVVEKKKKSINQNNA |
| | | DIQVVNAISSLTYPGALVKANSELVENQPDVLPVKRDSLTLSIDL |
| | | PGMTNQDNKIVVKNATKSNVNNAVNTLVERWNEKYAQAYPNVSAK |
| | | IDYDDEMAYSESQLIAKFGTAFKAVNNSLNVNFGAISEGKMQEEV |
| | | ISFKQIYYNVNVNEPTRPSRFFGKAVTKEQLQALGVNAENPPAYI |
| | | SSVAYGRQVYLKLSTNSHSTKVKAAFDAAVSGKSVSGDVELTNII |
| | | KNSSFKAVIYGGSAKDEVQIIDGNLGDLRDILKKGATFNRETPGV |
| | | PIAYTTNFLKDNELAVIKNNSEYIETTSKAYTDGKINIDHSGGYV |
| | | AQFNISWDEVNYDPEGNEIVQHKNWSENNKSKLAHFTSSIYLPGN |
| | | ARNINVYAKECTGLAWEWWRTVIDDRNLPLVKNRNISIWGTTLYP |
| | | KYSNKVDNPIE (SEQ ID NO: 2) |
| streptolysin O | BAB41212.2 | MSNKKTFKKYSRVAGLLTAALIIGNLVTANAESNKQNTASTETTT |
| (Streptococcus | GI: 71061060 | TSEQPKPESSELTIEKAGQKMDDMLNSNDMIKLAPKEMPLESAEK |
| pyogenes) | | EEKKSEDKKKSEEDHTEEINDKIYSLNYNELEVLAKNGETIENFV |
| | | PKEGVKKADKFIVIERKKKNINTTPVDISIIDSVTDRTYPAALQL |
| | | ANKGFTENKPDAVVTKRNPQKIHIDLPGMGDKATVEVNDPTYANV |
| | | STAIDNLVNQWHDNYSGGNTLPARTQYTESMVYSKSQIEAALNVN |
| | | SKILDGTLGIDFKSISKGEKKVMIAAYKQIFYTVSANLPNNPADV |
| | | FDKSVTFKDLQRKGVSNEAPPLFVSNVAYGRTVFVKLETSSKSND |
| | | VEAAFSAALKGTDVKTNGKYSDILENSSFTAVVLGGDAAEHNKVV |
| | | TKDFDVIRNVIKDNATFSRKNPAYPISYTSVFLKNNKIAGVNNRT |
| | | EYVETTSTEYTSGKINLSHQGAYVAQYEILWDEINYDDKGKEVIT |
| | | KRRWDNNWYSKTSPFSTVIPLGANSRNIRIMARECTGLAWEWWRK |
| | | VIDERDVKLSKEINVNISGSTLSPYGSITYK |
| | | (SEQ ID NO: 3) |
| perfringolysin O | NP_561079.1 | MIRFKKTKLIASIAMALCLFSQPVISFSKDITDKNQSIDSGISSL |
| (Clostridium | GI: 18309145 | SYNRNEVLASNGDKIESFVPKEGKKTGNKFIVVERQKRSLTTSPV |
| perfringens) | | DISIIDSVNDRTYPGALQLADKAFVENRPTILMVKRKPININIDL |
| | | PGLKGENSIKVDDPTYGKVSGAIDELVSKWNEKYSSTHTLPARTQ |
| | | YSESMVYSKSQISSALNVNAKVLENSLGVDFNAVANNEKKVMILA |
| | | YKQIFYTVSADLPKNPSDLFDDSVTFNDLKQKGVSNEAPPLMVSN |
| | | VAYGRTIYVKLETTSSSKDVQAAFKALIKNTDIKNSQQYKDIYEN |
| | | SSFTAVVLGGDAQEHNKVVTKDFDEIRKVIKDNATFSTKNPAYPI |
| | | SYTSVFLKDNSVAAVHNKTDYIETTSTEYSKGKINLDHSGAYVAQ |
| | | FEVAWDEVSYDKEGNEVLTHKTWDGNYQDKTAHYSTVIPLEANAR |
| | | NIRIKARECTGLAWEWWRDVISEYDVPLTNNINVSIWGTTLYPGS |
| | | SITYN (SEQ ID NO: 4) |
| Pneumolysin | NP_359331.1 | MANKAVNDFILAMNYDKKKLLTHQGESIENRFIKEGNQLPDEFVV |
| GI: 933687 | | IERKKRSLSTNTSDISVTATNDSRLYPGALLVVDETLLENNPTLL |
| (Streptococcus | | AVDRAPMTYSIDLPGLASSDSFLQVEDPSNSSVRGAVNDLLAKWH |
| pneumoniae) | | QDYGQVNNVPARMQYEKITAHSMEQLKVKFGSDFEKTGNSLDIDF |
| | | NSVHSGEKQIQIVNFKQIYYTVSVDAVKNPGDVFQDTVTVEDLKQ |
| | | RGISAERPLVYISSVAYGRQVYLKLETTSKSDEVEAAFEALIKGV |
| | | KVAPQTEWKQILDNTEVKAVILGGDPSSGARVVTGKVDMVEDLIQ |
| | | EGSRFTADHPGLPISYTTSFLRDNVVATFQNSTDYVETKVTAYRN |
| | | GDLLLDHSGAYVAQYYITWDELSYDHQGKEVLTPKAWDRNGQDLT |
| | | AHFTTSIPLKGNVRNLSVKIRECTGLAWEWWRTVYEKTDLPLVRK |
| | | RTISIWGTTLYPQVEDKVEND (SEQ ID NO: 5) |

In some embodiments, a library member (e.g., a library member which is a bacterial cell) includes an invasin that facilitates uptake by the antigen presenting cell. In some embodiments, a library member includes an autolysin that facilitates autolysis of the library member within the antigen presenting cell. In some embodiments, a library member includes both an invasin and an autolysin. In some embodiments, a library member which is an E. coli cell includes an invasin and/or an autolysin. In various embodiments, library cells that express an invasin and/or autolysin are used in methods that also employ non-professional antigen presenting cells or antigen presenting cells that are from cell lines. Isberg et al. (Cell, 1987, 50:769-778), Sizemore et al. (Science, 1995, 270:299-302) and Courvalin et al. (C.R. Acad. Sci. Paris, 1995, 318:1207-12) describe expression of an invasin to effect endocytosis of bacteria by target cells. Autolysins are described by Cao et al., Infect. Immun. 1998, 66(6): 2984-2986; Margot et al., J. Bacteriol. 1998, 180(3):749-752; Buist et al., Appl. Environ. Microbiol., 1997, 63(7):2722-2728; Yamanaka et al., FEMS Microbiol. Lett., 1997, 150(2): 269-275; Romero et al., FEMS Microbiol. Lett., 1993, 108(1):87-92; Betzner and Keck, Mol. Gen. Genet., 1989, 219(3): 489-491; Lubitz et al., J. Bacteriol., 1984, 159(1):385-387; and Tomasz et al., J. Bacteriol., 1988, 170(12): 5931-5934. In some embodiments, an autolysin has a feature that permits delayed lysis, e.g., the autolysin is temperature-sensitive or time-sensitive (see, e.g., Chang et al., 1995, J. Bact. 177, 3283-3294; Raab et al., 1985, J. Mol. Biol. 19, 95-105; Gerds et al., 1995, Mol. Microbiol. 17, 205-210). Useful cytolysins also include addiction (poison/antidote) autolysins, (see, e.g., Magnuson R, et al., 1996, J. Biol. Chem. 271(31), 18705-18710; Smith A S, et al., 1997, Mol. Microbiol. 26(5), 961-970).

In some embodiments, members of the library include bacterial cells. In certain embodiments, the library includes non-pathogenic, non-virulent bacterial cells. Examples of bacteria for use as library members include E. coli, mycobacteria, Listeria monocytogenes, Shigella flexneri, Bacillus subtilis, or Salmonella.

In some embodiments, members of the library include eukaryotic cells (e.g., yeast cells). In some embodiments, members of the library include viruses (e.g., bacteriophages). In some embodiments, members of the library include liposomes. Methods for preparing liposomes that include a cytolysin and other agents are described in Kyung-Dall et al., U.S. Pat. No. 5,643,599. In some embodiments, members of the library include beads. Methods for preparing libraries comprised of beads are described, e.g., in Lam et al., Nature 354: 82-84, 1991, U.S. Pat. Nos. 5,510,240 and 7,262,269, and references cited therein.

In certain embodiments, a library is constructed by cloning polynucleotides encoding polypeptides of interest, or portions thereof, into vectors that express the polypeptides of interest in cells of the library. The polynucleotides can be synthetically synthesized. The polynucleotides can be cloned by designing primers that amplify the polynucleotides. Primers can be designed using available software, such as Primer3Plus (available the following URL: bioinformatics.nl/cgi-bin/primer3plus/primer3plus.cgi; see Rozen and Skaletsky, In: Krawetz S, Misener S (eds) Bioinformatics Methods and Protocols: Methods in Molecular Biology. Humana Press, Totowa, N.J., pp. 365-386, 2000). Other methods for designing primers are known to those of skill in the art. In some embodiments, primers are constructed so as to produce polypeptides that are truncated, and/or lack hydrophobic regions (e.g., signal sequences or transmembrane regions) to promote efficient expression. The location of predicted signal sequences and predicted signal sequence cleavage sites in a given open reading frame (ORF) sequence can be determined using available software, see, e.g., Dyrlov et al., J. Mol. Biol., 340:783-795, 2004, and the following URL: cbs.dtu.dk/services/SignalP/). For example, if a signal sequence is predicted to occur at the N-terminal 20 amino acids of a given polypeptide sequence, a primer is designed to anneal to a coding sequence downstream of the nucleotides encoding the N-terminal 20 amino acids, such that the amplified sequence encodes a product lacking this signal sequence.

Primers can also be designed to include sequences that facilitate subsequent cloning steps. ORFs can be amplified directly from genomic DNA (e.g., genomic DNA of a tumor cell), or from polynucleotides produced by reverse transcription (RT-PCR) of mRNAs expressed by the tumor cell. RT-PCR of mRNA is useful, e.g., when the genomic sequence of interest contains intronic regions. PCR-amplified ORFs are cloned into an appropriate vector, and size, sequence, and expression of ORFs can be verified prior to use in immunological assays.

In some embodiments, a polynucleotide encoding a polypeptide of interest is linked to a sequence encoding a tag (e.g., an N-terminal or C-terminal epitope tag) or a reporter protein (e.g., a fluorescent protein). Epitope tags and reporter proteins facilitate purification of expressed polypeptides, and can allow one to verify that a given polypeptide is properly expressed in a library host cell, e.g., prior to using the cell in a screen. Useful epitope tags include, for example, a polyhistidine (His) tag, a V5 epitope tag from the P and V protein of paramyxovirus, a hemagglutinin (HA) tag, a myc tag, and others. In some embodiments, a polynucleotide encoding a polypeptide of interest is fused to a sequence encoding a tag which is a known antigenic epitope (e.g., an MHC class I- and/or MHC class II-restricted T cell epitope of a model antigen such as an ovalbumin), and which can be used to verify that a polypeptide of interest is expressed and that the polypeptide-tag fusion protein is processed and presented in antigen presentation assays. In some embodiments a tag includes a T cell epitope of a murine T cell (e.g., a murine T cell line). In some embodiments, a polynucleotide encoding a polypeptide of interest is linked to a tag that facilitates purification and a tag that is a known antigenic epitope. Useful reporter proteins include naturally occurring fluorescent proteins and their derivatives, for example, Green Fluorescent Protein (Aequorea Victoria) and Neon Green (Branchiostoma lanceolatum). Panels of synthetically derived fluorescent and chromogenic proteins are also available from commercial sources.

Polynucleotides encoding a polypeptide of interest are cloned into an expression vector for introduction into library host cells. Various vector systems are available to facilitate cloning and manipulation of polynucleotides, such as the Gateway® Cloning system (Invitrogen). As is known to those of skill in the art, expression vectors include elements that drive production of polypeptides of interest encoded by a polynucleotide in library host cells (e.g., promoter and other regulatory elements). In some embodiments, polypeptide expression is controlled by an inducible element (e.g., an inducible promoter, e.g., an IPTG- or arabinose-inducible promoter, or an IPTG-inducible phage T7 RNA polymerase system, a lactose (lac) promoter, a tryptophan (trp) promoter, a tac promoter, a trc promoter, a phage lambda promoter, an alkaline phosphatase (phoA) promoter, to give just a few examples; see Cantrell, Meth. in Mol. Biol., 235:257-276, Humana Press, Casali and Preston, Eds.). In some embodiments, polypeptides are expressed as cytoplasmic polypeptides. In some embodiments, the vector used for polypeptide expression is a vector that has a high copy number in a library host cell. In some embodiments, the vector used for expression has a copy number that is more than 25, 50, 75, 100, 150, 200, or 250 copies per cell. In some embodiments, the vector used for expression has a ColE1 origin of replication. Useful vectors for polypeptide expression in bacteria include pET vectors (Novagen), Gateway© pDEST vectors (Invitrogen), pGEX vectors (Amersham Biosciences), pPRO vectors (BD Biosciences), pBAD vectors (Invitrogen), pLEX vectors (Invitrogen), pMAL™ vectors (New England BioLabs), pGEMEX vectors (Promega), and pQE vectors (Qiagen). Vector systems for producing phage libraries are known and include Novagen T7Select® vectors, and New England Biolabs Ph.D.™ Peptide Display Cloning System.

In some embodiments, library host cells express (either constitutively, or when induced, depending on the selected expression system) a polypeptide of interest to at least 10%, 20%, 30%, 40%, 50%, 60%, or 70% of the total cellular protein. In some embodiments, the level a polypeptide available in or on a library member (e.g., cell, virus particle, liposome, bead) is such that antigen presenting cells exposed to a sufficient quantity of the library members are presented on MHC molecules polypeptide epitopes at a density that is comparable to the density presented by antigen presenting cells pulsed with purified peptides.

Methods for efficient, large-scale production of libraries are available. For example, site-specific recombinases or rare-cutting restriction enzymes can be used to transfer polynucleotides between expression vectors in the proper orientation and reading frame (Walhout et al., Meth. Enzymol. 328:575-592, 2000; Marsischky et al., Genome Res. 14.2020-202, 2004; Blommel et al., Protein Expr. Purif 47:562-570, 2006).

For production of liposome libraries, expressed polypeptides (e.g., purified or partially purified polypeptides) can be entrapped in liposomal membranes, e.g., as described in Wassef et al., U.S. Pat. No. 4,863,874; Wheatley et al., U.S. Pat. No. 4,921,757; Huang et al., U.S. Pat. No. 4,925,661; or Martin et al., U.S. Pat. No. 5,225,212.

A library can be designed to include full length polypeptides and/or portions of polypeptides. Expression of full length polypeptides maximizes epitopes available for presentation by a human antigen presenting cell, thereby increasing the likelihood of identifying an antigen. However, in some embodiments, it is useful to express portions of polypeptides, or polypeptides that are otherwise altered, to achieve efficient expression. For example, in some embodiments, polynucleotides encoding polypeptides that are large (e.g., greater than 1,000 amino acids), that have extended hydrophobic regions, signal peptides, transmembrane domains, or domains that cause cellular toxicity, are modified (e.g., by C-terminal truncation, N-terminal truncation, or internal deletion) to reduce cytotoxicity and permit efficient expression a library cell, which in turn facilitates presentation of the encoded polypeptides on human cells. Other types of modifications, such as point mutations or codon optimization, may also be used to enhance expression.

The number of polypeptides included in a library can be varied. For example, in some embodiments, a library can be designed to express polypeptides from at least 5%, 10%, 15%, 20%, 25%, 35%, 40%, 45%, 50%, 55%, 60%, 70%, 75%, 80%, 85%, 90%, 95%, 97%, 98%, 99%, or more, of ORFs in a target cell (e.g., tumor cell). In some embodiments, a library expresses at least 10, 15, 20, 25, 30, 40, 50, 75, 100, 150, 200, 250, 300, 350, 400, 450, 500, 550, 600, 650, 700, 750, 800, 850, 900, 950, 1000, 2500, 5000, 10,000, or more different polypeptides of interest, each of which may represent a polypeptide encoded by a single full length polynucleotide or portion thereof.

In some embodiments, assays may focus on identifying antigens that are secreted polypeptides, cell surface-expressed polypeptides, or virulence determinants, e.g., to identify antigens that are likely to be targets of both humoral and cell mediated immune responses.

In addition to polypeptides of interest, libraries can include tags or reporter proteins that allow one to easily purify, analyze, or evaluate MHC presentation, of the polypeptide of interest. In some embodiments, polypeptides expressed by a library include C-terminal tags that include both an MHC class I and an MHC class II-restricted T cell epitope from a model antigen, such as chicken ovalbumin (OVA). Library protein expression and MIIC presentation is validated using these epitopes. In some embodiments, the epitopes are OVA₂₄₇₋₂₆₅ and OVA₂₅₈₋₂₆₅ respectfully, corresponding to positions in the amino acid sequence found in GenBank® under Acc. No. NP_990483. Expression and presentation of linked ORFs can be verified with antigen presentation assays using T cell hybridomas (e.g., B3Z T hybridoma cells, which are H2-K^{b} restricted, and KZO T hybridoma cells, which are H2-A^{k} restricted) that specifically recognize these epitopes.

Sets of library members (e.g., bacterial cells) can be provided on an array (e.g., on a solid support, such as a 96-well plate) and separated such that members in each location express a different polypeptide of interest, or a different set of polypeptides of interest.

Methods of using library members for identifying T cell antigens are described in detail below. In addition to these methods, library members also have utility in assays to identify B cell antigens. For example, lysate prepared from library members that include polypeptides of interest can be used to screen a sample comprising antibodies (e.g., a serum sample) from a subject (e.g., a subject who has been exposed to an infectious agent of interest, a subject who has cancer, and/or a control subject), to determine whether antibodies present in the subject react with the polypeptide of interest. Suitable methods for evaluating antibody reactivity are known and include, e.g., ELISA assays.

### Polypeptides of Interest

In some embodiments, methods and compositions described herein can be used to identify and/or detect immune responses to a polypeptide of interest. In some embodiments, a polypeptide of interest is encoded by an ORF from a target tumor cell, and members of a library include (e.g., internally express or carry) ORFs from a target tumor cell. In some such embodiments, a library can be used in methods described herein to assess immune responses to one or more polypeptides of interest encoded by one or more ORFs. In some embodiments, methods of the disclosure identify one or more polypeptides of interest as stimulatory antigens (e.g., that stimulate an immune response, e.g., a T cell response, e.g., expression and/or secretion of one or more immune mediators). In some embodiments, methods of the disclosure identify one or more polypeptides of interest as antigens or potential antigens that have minimal or no effect on an immune response (e.g., expression and/or secretion of one or more immune mediators). In some embodiments, methods of the disclosure identify one or more polypeptides of interest as inhibitory and/or suppressive antigens (e.g., that inhibit, suppress, down-regulate, impair, and/or prevent an immune response, e.g., a T cell response, e.g., expression and/or secretion of one or more immune mediators). In some embodiments, methods of the disclosure identify one or more polypeptides of interest as tumor antigens or potential tumor antigens, e.g., tumor specific antigens (TSAs, or neoantigens), tumor associated antigens (TAAs), or cancer/testis antigens (CTAs).

In some embodiments, a polypeptide of interest is a putative tumor antigen, and methods and compositions described herein can be used to identify and/or detect immune responses to one or more putative tumor antigens. For example, members of a library include (e.g., internally express or carry) putative tumor antigens (e.g., a polypeptide previously identified (e.g., by a third party) as a tumor antigen, e.g., identified as a tumor antigen using a method other than a method of the present disclosure). In some embodiments, a putative tumor antigen is a tumor antigen described herein. In some such embodiments, such libraries can be used to assess whether and/or the extent to which such putative tumor antigen mediates an immune response. In some embodiments, methods of the disclosure identify one or more putative tumor antigens as stimulatory antigens. In some embodiments, methods of the disclosure identify one or more putative tumor antigens as antigens that have minimal or no effect on an immune response. In some embodiments, methods of the disclosure identify one or more putative tumor antigens as inhibitory and/or suppressive antigens.

In some embodiments, a polypeptide of interest is a pre-selected tumor antigen, and methods and compositions described herein can be used to identify and/or detect immune responses to one or more pre-selected tumor antigens. For example, in some embodiments, members of a library include (e.g., internally express or carry) one or more polypeptides identified as tumor antigens using a method of the present disclosure and/or using a method other than a method of the present disclosure. In some such embodiments, such libraries can be used to assess whether and/or the extent to which such tumor antigens mediate an immune response by an immune cell from one or more subjects (e.g., a subject who has cancer and/or a control subject) to obtain one or more response profiles described herein. In some embodiments, methods of the disclosure identify one or more pre-selected tumor antigens as stimulatory antigens for one or more subjects. In some embodiments, methods of the disclosure identify one or more pre-selected tumor antigens as antigens that have minimal or no effect on an immune response for one or more subjects. In some embodiments, methods of the disclosure identify one or more pre-selected tumor antigens as inhibitory and/or suppressive antigens for one or more subjects.

In some embodiments, a polypeptide of interest is a known tumor antigen, and methods and compositions described herein can be used to identify and/or detect immune responses to one or more known tumor antigens. For example, in some embodiments, members of a library include (e.g., internally express or carry) one or more polypeptides identified as a tumor antigen using a method of the present disclosure and/or using a method other than a method of the present disclosure. In some such embodiments, such libraries can be used to assess whether and/or the extent to which such tumor antigens mediate an immune response by an immune cell from one or more subjects (e.g., a subject who has cancer and/or a control subject) to obtain one or more response profiles described herein. In some embodiments, methods of the disclosure identify one or more known tumor antigens as stimulatory antigens for one or more subjects. In some embodiments, methods of the disclosure identify one or more known tumor antigens as antigens that have minimal or no effect on an immune response for one or more subjects. In some embodiments, methods of the disclosure identify one or more known tumor antigens as inhibitory and/or suppressive antigens for one or more subjects.

In some embodiments, a polypeptide of interest is a potential tumor antigen, and methods and compositions described herein can be used to identify and/or detect immune responses to one or more potential tumor antigens. For example, in some embodiments, members of a library include (e.g., internally express or carry) one or more polypeptides identified as being of interest, e.g., encoding mutations associated with a tumor, using a method of the present disclosure and/or using a method other than a method of the present disclosure. In some such embodiments, such libraries can be used to assess whether and/or the extent to which such polypeptides mediate an immune response by an immune cell from one or more subjects (e.g., a subject who has cancer and/or a control subject) to obtain one or more response profiles described herein. In some embodiments, methods of the disclosure identify one or more polypeptides as stimulatory antigens for one or more subjects. In some embodiments, methods of the disclosure identify one or more polypeptides as antigens that have minimal or no effect on an immune response for one or more subjects. In some embodiments, methods of the disclosure identify one or more polypeptides as inhibitory and/or suppressive antigens for one or more subjects.

### Tumor Antigens

Polypeptides of interest used in methods and systems described herein include tumor antigens and potential tumor antigens, e.g., tumor specific antigens (TSAs, or neoantigens), tumor associated antigens (TAAs), and/or cancer/testis antigens (CTAs). Exemplary tumor antigens include, e.g., MART-1/MelanA (MART-I or MLANA), gp100 (Pmel 17 or SILV), tyrosinase, TRP-1, TRP-2, MAGE-1, MAGE-3 (also known as HIP8), BAGE, GAGE-1, GAGE-2, p15, Calcitonin, Calretinin, Carcinoembryonic antigen (CEA), Chromogranin, Cytokeratin, Desmin, Epithelial membrane protein (EMA), Factor VIII, Glial fibrillary acidic protein (GFAP), Gross cystic disease fluid protein (GCDFP-15), HMB-45, Human chorionic gonadotropin (hCG), inhibin, lymphocyte marker, MART-1 (Melan-A), Myo D1, muscle-specific actin (MSA), neurofilament, neuron-specific enolase (NSE), placental alkaline phosphatase (PLAP), prostate-specific antigen, PTPRC (CD45), S100 protein, smooth muscle actin (SMA), synaptophysin, thyroglobulin, thyroid transcription factor-1, Tumor M2-PK, vimentin, p53, Ras, HER-2/neu, BCR-ABL, E2A-PRL, H4-RET, IGH-IGK, MYL-RAR, Epstein Barr virus antigens (e.g., EBNA1), human papillomavirus (HPV) antigen E6 or E7 (HPV_E6 or HPV_E7), TSP-180, MAGE-4, MAGE-5, MAGE-6, RAGE, NY-ESO-1 (also known as CTAGIB), erbB, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein (AFP), beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, MUC16, IL13Ra2, FRα, VEGFR2, Lewis Y, FAP, EphA2, CEACAM5, EGFR, CA6, CA9, GPNMB, EGP1, FOLR1, endothelial receptor, STEAPI, SLC44A4, Nectin-4, AGS-16, guanalyl cyclase C, MUC-1, CFC1B, integrin alpha 3 chain (of a3b1, a laminin receptor chain), TPS, CD19, CD20, CD22, CD30, CD31, CD72, CD180, CD171 (LICAM), CD123, CD133, CD138, CD37, CD70, CD79a, CD79b, CD56, CD74, CD166, CD71, CD34, CD99, CD117, CD80, CD28, CD13, CD15, CD25, CD10, CLL-1/CLEC12A, RORI, Glypican 3 (GPC3), Mesothelin, CD33/IL3Ra, c-Met, PSCA, PSMA, Glycolipid F77, EGFRvIII, BCMA, GD-2, PSAP, prostein (also known as P501S), PSMA, Survivin (also known as BIRC5), and MAGE-A3, MAGEA2, MAGEA4, MAGEA6, MAGEA9, MAGEA10, MAGEA12, BIRC5, CDH3, CEACAM3, CGB_isoform2, ELK4, ERBB2, HPSE1, HPSE2, KRAS_isoform1, KRAS_isoform2, MUC1, SMAD4, TERT,2. TERT.3, TGFBR2, EGAG9_isoform1, TP53, CGB_isoform1, IMPDH2, LCK, angiopoietin-1 (Ang1) (also known as ANGPT1), XIAP (also known as BIRC4), galectin-3 (also known as LGALS3), VEGF-A (also known as VEGF), ATP6S1 (also known as ATP6AP1), MAGE-A1, cIAP-1 (also known as BIRC2), macrophage migration inhibitory factor (MIF), galectin-9 (also known as LGALS9), progranulin PGRN (also known as granulin), OGFR, MLIAP (also known as BIRC7), TBX4 (also known as ICPPS, SPS or T-Box4), secretory leukocyte protein inhibitor (Slpi) (also known as antileukoproteinase), Ang2 (also known as ANGPT2), galectin-1 (also known as LGALS1), TRP-2 (also known as DCT), hTERT (telomerase reverse transcriptase) tyrosinase-related protein 1 (TRP-1, TYRP1), NOR-90/UBF-2 (also known as UBTF), LGMN, SPA17, PRTN3, TRRAP_1, TRRAP_2, TRRAP 3, TRRAP 4, MAGEC2, PRAME, SOX10, RAC1, HRAS, GAGE4, AR, CYP1B1, MMP8, TYR, PDGFRB, KLK3, PAX3, PAX5, ST3GAL5, PLACI, RhoC, MYCN, REG3A, CSAG2, CTAG2-1a, CTAG2-1b, PAGE4, BRAF, GRM3, ERBB4, KIT, MAPK1, MFI2, SART3, ST8SIA1, WDR46, AKAP-4, RGS5, FOSL1, PRM2, ACRBP, CTCFL, CSPG4, CCNB1, MSLN, WT1, SSX2, KDR, ANKRD30A, MAGED1, MAP3K9, XAGE1B, PREX2, CD276, TEK, AIM1, ALK, FOLH1, GRIN2A MAP3K5 and one or more isoforms of any preceding tumor antigens. Exemplary tumor antigens are provided in the accompanying list of sequences.

Tumor specific antigens (TSAs, or neoantigens) are tumor antigens that are not encoded in normal host genome (see, e.g., Yarchoan et al., Nat. Rev. Cancer. 2017 Feb. 24. doi: 10.1038/nrc.2016.154; Gubin et al., J. Clin. Invest. 125:3413-3421 (2015)). In some embodiments, TSAs arise from somatic mutations and/or other genetic alterations. In some embodiments, TSAs arise from missense or in-frame mutations. In some embodiments, TSAs arise from frame-shift mutations or loss-of-stop-codon mutations. In some embodiments, TSAs arise from insertion or deletion mutations. In some embodiments, TSAs arise from duplication or repeat expansion mutations. In some embodiments, TSAs arise from splice variants or improper splicing. In some embodiments, TSAs arise from gene fusions. In some embodiments, TSAs arise from translocations. In some embodiments, TSAs include oncogenic viral proteins. For example, as with Merkel cell carcinoma (MCC) associated with the Merkel cell polyomavirus (MCPyV) and cancers of the cervix, oropharynx and other sites associated with the human papillomavirus (HPV), TSAs include proteins encoded by viral open reading frames. For purposes of this disclosure, the terms “mutation” and “mutations” encompass all mutations and genetic alterations that may give rise to an antigen encoded in the genome of a cancer or tumor cell of a subject, but not in a normal or non-cancerous cell of the same subject. In some embodiments, TSAs are specific (personal) to a subject. In some embodiments, TSAs are shared by more than one subject, e.g., less than 1%, 1-3%, 1-5%, 1-10%, or more of subjects suffering from a cancer. In some embodiments, TSAs shared by more than one subject may be known or pre-selected.

In some embodiments, a TSA is encoded by an open reading frame from a virus. For example, a library can be designed to express polypeptides from one of the following viruses: an immunodeficiency virus (e.g., a human immunodeficiency virus (HIV), e.g., HIV-1, HIV-2), a hepatitis virus (e.g., hepatitis B virus (HBV), hepatitis C virus (HCV), hepatitis A virus, non-A and non-B hepatitis virus), a herpes virus (e.g., herpes simplex virus type I (HSV-1), HSV-2, Varicella-zoster virus, Epstein Barr virus, human cytomegalovirus, human herpesvirus 6 (HHV-6), HHV-7, HHV-8), a poxvirus (e.g., variola, vaccinia, monkeypox, Molluscum contagiosum virus), an influenza virus, a human papilloma virus, adenovirus, rhinovirus, coronavirus, respiratory syncytial virus, rabies virus, coxsackie virus, human T cell leukemia virus (types I, II and III), parainfluenza virus, paramyxovirus, poliovirus, rotavirus, rhinovirus, rubella virus, measles virus, mumps virus, adenovirus, yellow fever virus, Norwalk virus, West Nile virus, a Dengue virus, Severe Acute Respiratory Syndrome Coronavirus (SARS-CoV), bunyavirus, Ebola virus, Marburg virus, Eastern equine encephalitis virus, Venezuelan equine encephalitis virus, Japanese encephalitis virus, St. Louis encephalitis virus, Junin virus, Lassa virus, and Lymphocytic choriomeningitis virus. Libraries for other viruses can also be produced and used according to methods described herein.

Tumor specific antigens are known in the art, any of which can be used in methods described herein. In some embodiments, gene sequences encoding polypeptides that are potential or putative neoantigens are determined by sequencing the genome and/or exome of tumor tissue and healthy tissue from a subject having cancer using next generation sequencing technologies. In some embodiments, genes that are selected based on their frequency of mutation and ability to encode a potential or putative neoantigen are sequenced using next-generation sequencing technology. Next-generation sequencing applies to genome sequencing, genome resequencing, transcriptome profiling (RNA-Seq), DNA-protein interactions (ChIP-sequencing), and epigenome characterization (de Magalhaes et al. (2010) Ageing Research Reviews 9 (3): 315-323; Hall N (2007) J. Exp. Biol. 209 (Pt 9): 1518-1525; Church (2006) Sci. Am. 294 (1): 46-54; ten Bosch et al. (2008) Journal of Molecular Diagnostics 10 (6): 484-492; Tucker T et al. (2009) The American Journal of Human Genetics 85 (2): 142-154). Next-generation sequencing can be used to rapidly reveal the presence of discrete mutations such as coding mutations in individual tumors, e.g., single amino acid changes (e.g., missense mutations, in-frame mutations) or novel stretches of amino acids generated by frame-shift insertions, deletions, gene fusions, read-through mutations in stop codons, duplication or repeat expansion mutations, and translation of splice variants or improperly spliced introns, and translocations (e.g., “neoORFs”).

Another method for identifying potential or putative neoantigens is direct protein sequencing. Protein sequencing of enzymatic digests using multidimensional MS techniques (MSn) including tandem mass spectrometry (MS/MS)) can also be used to identify neoantigens. Such proteomic approaches can be used for rapid, highly automated analysis (see, e.g., Gevaert et al., Electrophoresis 21:1145-1154 (2000)). High-throughput methods for de novo sequencing of unknown proteins can also be used to analyze the proteome of a subject's tumor to identify expressed potential or putative neoantigens. For example, meta shotgun protein sequencing may be used to identify expressed potential or putative neoantigens (see e.g., Guthals et al. (2012) Molecular and Cellular Proteomics 11(10):1084-96).

Potential or putative neoantigens may also be identified using MHC multimers to identify neoantigen-specific T cell responses. For example, high-throughput analysis of neoantigen-specific T cell responses in patient samples may be performed using MHC tetramer-based screening techniques (see e.g., Hombrink et al. (2011) PLoS One; 6(8): e22523; Hadrup et al. (2009) Nature Methods, 6(7):520-26; van Rooij et al. (2013) Journal of Clinical Oncology, 31:1-4; and Heemskerk et al. (2013) EMBO Journal, 32(2):194-203).

In some embodiments, one or more known or pre-selected tumor specific antigens, or one or more potential or putative tumor specific antigens identified using one of these methods, can be included in a library described herein.

Tumor associated antigens (TAAs) include proteins encoded in a normal genome (see, e.g., Ward et al., Adv. Immunol. 130:25-74 (2016)). In some embodiments, TAAs are either normal differentiation antigens or aberrantly expressed normal proteins. Overexpressed normal proteins that possess growth/survival-promoting functions, such as Wilms tumor 1 (WT1) (Ohminami et al., Blood 95:286-293 (2000)) or Her2/neu (Kawashima et al., Cancer Res. 59:431-435 (1999)), are TAAs that directly participate in the oncogenic process. Post-translational modifications, such as phosphorylation, of proteins may also lead to formation of TAAs (Doyle, J. Biol. Chem. 281:32676-32683 (2006); Cobbold, Sci. Transl. Med. 5:203ra125 (2013)). TAAs are generally shared by more than one subject, e.g., less than 1%, 1-3%, 1-5%, 1-10%, 1-20%, or more of subjects suffering from a cancer. In some embodiments, TAAs are known or pre-selected tumor antigens. In some embodiments, with respect to an individual subject, TAAs are potential or putative tumor antigens. Cancer/testis antigens (CTAs) are expressed by various tumor types and by reproductive tissues (for example, testes, fetal ovaries and trophoblasts) but have limited or no detectable expression in other normal tissues in the adult and are generally not presented on normal reproductive cells, because these tissues do not express MHC class I molecules (see, e.g., Coulie et al., Nat. Rev. Cancer 14:135-146 (2014); Simpson et al., Nat. Rev. Cancer 5:615-625 (2005); Scanlan et al., Immunol. Rev. 188:22-32 (2002)).

### Human Cells for Antigen Presentation

Methods of the present disclosure utilize human antigen presenting cells. Human antigen presenting cells express ligands for antigen receptors and other immune activation molecules on human lymphocytes. Given differences in MHC peptide binding specificities and antigen processing enzymes between species, antigens processed and presented by human cells are more likely to be physiologically relevant human antigens in vivo than antigens identified in non-human systems. Accordingly, methods of identifying these antigens employ human cells to present candidate tumor antigen polypeptides. Any human cell that internalizes library members and presents polypeptides expressed by the library members on MHC molecules can be used as an antigen presenting cell according to the present disclosure. In some embodiments, human cells used for antigen presentation are primary human cells. The cells can include peripheral blood mononuclear cells (PBMC) of a human. In some embodiments, peripheral blood cells are separated into subsets (e.g., subsets comprising dendritic cells, macrophages, monocytes, B cells, or combinations thereof) prior to use in an antigen presentation assay. In some embodiments, a subset of cells that expresses MHC class II is selected from peripheral blood. In one example, a cell population including dendritic cells is isolated from peripheral blood. In some embodiments, a subset of dendritic cells is isolated (e.g., plasmacytoid, myeloid, or a subset thereof). Human dendritic cell markers include CD1c, CD1a, CD303, CD304, CD141, and CD209. Cells can be selected based on expression of one or more of these markers (e.g., cells that express CD303, CD1c, and CD141).

Dendritic cells can be isolated by positive selection from peripheral blood using commercially available kits (e.g., from Miltenyi Biotec Inc.). In some embodiments, the dendritic cells are expanded ex vivo prior to use in an assay. Dendritic cells can also be produced by culturing peripheral blood cells under conditions that promote differentiation of monocyte precursors into dendritic cells in vitro. These conditions typically include culturing the cells in the presence of cytokines such as GM-CSF and IL-4 (see, e.g., Inaba et al., Isolation of dendritic cells, Curr. Protoc. Immunol. May; Chapter 3: Unit 3.7, 2001). Procedures for in vitro expansion of hematopoietic stem and progenitor cells (e.g., taken from bone marrow or peripheral blood), and differentiation of these cells into dendritic cells in vitro, is described in U.S. Pat. No. 5,199,942, and U.S. Pat. Pub. 20030077263. Briefly, CD34⁺ hematopoietic stem and progenitor cells are isolated from peripheral blood or bone marrow and expanded in vitro in culture conditions that include one or more of Flt3-L, IL-1, IL-3, and c-kit ligand.

In some embodiments, immortalized cells that express human MHC molecules (e.g., human cells, or non-human cells that are engineered to express human MHC molecules) are used for antigen presentation. For example, assays can employ COS cells transfected with human MHC molecules or HeLa cells.

In some embodiments, both the antigen presenting cells and immune cells used in the method are derived from the same subject (e.g., autologous T cells and APC are used). In these embodiments, it can be advantageous to sequentially isolate subsets of cells from peripheral blood of the subject, to maximize the yield of cells available for assays. For example, one can first isolate CD4⁺ and CD8⁺ T cell subsets from the peripheral blood. Next, dendritic cells (DC) are isolated from the T cell-depleted cell population. The remaining T- and DC-depleted cells are used to supplement the DC in assays, or are used alone as antigen presenting cells. In some embodiments, DC are used with T- and DC-depleted cells in an assay, at a ratio of 1:2, 1:3, 1:4, or 1:5. In some embodiments, the antigen presenting cells and immune cells used in the method are derived from different subjects (e.g., heterologous T cells and APC are used).

Antigen presenting cells can be isolated from sources other than peripheral blood. For example, antigen presenting cells can be taken from a mucosal tissue (e.g., nose, mouth, bronchial tissue, tracheal tissue, the gastrointestinal tract, the genital tract (e.g., vaginal tissue), or associated lymphoid tissue), peritoneal cavity, lymph nodes, spleen, bone marrow, thymus, lung, liver, kidney, neuronal tissue, endocrine tissue, or other tissue, for use in screening assays. In some embodiments, cells are taken from a tissue that is the site of an active immune response (e.g., an ulcer, sore, or abscess). Cells may be isolated from tissue removed surgically, via lavage, or other means.

Antigen presenting cells useful in methods described herein are not limited to “professional” antigen presenting cells. In some embodiments, non-professional antigen presenting cells can be utilized effectively in the practice of methods of the present disclosure. Non-professional antigen presenting cells include fibroblasts, epithelial cells, endothelial cells, neuronal/glial cells, lymphoid or myeloid cells that are not professional antigen presenting cells (e.g., T cells, neutrophils), muscle cells, liver cells, and other types of cells.

Antigen presenting cells are cultured with library members that express a polypeptide of interest (and, if desired, a cytolysin polypeptide) under conditions in which the antigen presenting cells internalize, process and present polypeptides expressed by the library members on MHC molecules. In some embodiments, library members are killed or inactivated prior to culture with the antigen presenting cells. Cells or viruses can be inactivated by any appropriate agent (e.g., fixation with organic solvents, irradiation, freezing). In some embodiments, the library members are cells that express ORFs linked to a tag (e.g., a tag which comprises one or more known T cell epitopes) or reporter protein, expression of which has been verified prior to the culturing.

In some embodiments, antigen presenting cells are incubated with library members at 37° C. for between 30 minutes and 5 hours (e.g., for 45 min. to 1.5 hours). After the incubation, the antigen presenting cells can be washed to remove library members that have not been internalized. In certain embodiments, the antigen presenting cells are non-adherent, and washing requires centrifugation of the cells. The washed antigen presenting cells can be incubated at 37° C. for an additional period of time (e.g., 30 min. to 2 hours) prior to exposure to lymphocytes, to allow antigen processing. In some embodiments, it is desirable to fix and kill the antigen presenting cells prior to exposure to lymphocytes (e.g., by treating the cells with 1% paraformaldehyde).

The antigen presenting cell and library member numbers can be varied, so long as the library members provide quantities of polypeptides of interest sufficient for presentation on MHC molecules. In some embodiments, antigen presenting cells are provided in an array, and are contacted with sets of library cells, each set expressing a different polypeptide of interest. In certain embodiments, each location in the array includes 1×10³-1×10⁶ antigen presenting cells, and the cells are contacted with 1×10³-1×10⁸ library cells which are bacterial cells.

In any of the embodiments described herein, antigen presenting cells can be freshly isolated, maintained in culture, and/or thawed from frozen storage prior to incubation with library cells, or after incubation with library cells.

### Human Lymphocytes

In methods of the present disclosure, human lymphocytes are tested for antigen-specific reactivity to antigen presenting cells, e.g., antigen presenting cells that have been incubated with libraries expressing polypeptides of interest as described above. The methods of the present disclosure permit rapid identification of human antigens using pools of lymphocytes isolated from an individual, or progeny of the cells. The detection of antigen-specific responses does not rely on laborious procedures to isolate individual T cell clones. In some embodiments, the human lymphocytes are primary lymphocytes. In some embodiments, human lymphocytes are NKT cells, gamma-delta T cells, or NK cells. Just as antigen presenting cells may be separated into subsets prior to use in antigen presentation assays, a population of lymphocytes having a specific marker or other feature can be used. In some embodiments, a population of T lymphocytes is isolated. In some embodiments, a population of CD4⁺ T cells is isolated. In some embodiments, a population of CD8⁺ T cells is isolated. CD8⁺ T cells recognize peptide antigens presented in the context of MHC class I molecules. Thus, in some embodiments, the CD8⁺ T cells are used with antigen presenting cells that have been exposed to library host cells that co-express a cytolysin polypeptide, in addition to a polypeptide of interest. T cell subsets that express other cell surface markers may also be isolated, e.g., to provide cells having a particular phenotype. These include CLA (for skin-homing T cells), CD25, CD30, CD69, CD154 (for activated T cells), CD45RO (for memory T cells), CD294 (for Th2 cells), γ/δ TCR-expressing cells, CD3 and CD56 (for NK T cells). Other subsets can also be selected.

Lymphocytes can be isolated, and separated, by any means known in the art (e.g., using antibody-based methods such as those that employ magnetic bead separation, panning, or flow cytometry). Reagents to identify and isolate human lymphocytes and subsets thereof are well known and commercially available.

Lymphocytes for use in methods described herein can be isolated from peripheral blood mononuclear cells, or from other tissues in a human. In some embodiments, lymphocytes are taken from tumors, lymph nodes, a mucosal tissue (e.g., nose, mouth, bronchial tissue, tracheal tissue, the gastrointestinal tract, the genital tract (e.g., vaginal tissue), or associated lymphoid tissue), peritoneal cavity, spleen, thymus, lung, liver, kidney, neuronal tissue, endocrine tissue, peritoneal cavity, bone marrow, or other tissues. In some embodiments, cells are taken from a tissue that is the site of an active immune response (e.g., an ulcer, sore, or abscess). Cells may be isolated from tissue removed surgically, via lavage, or other means.

Lymphocytes taken from an individual can be maintained in culture or frozen until use in antigen presentation assays. In some embodiments, freshly isolated lymphocytes can be stimulated in vitro by antigen presenting cells exposed to library cells as described above. In some embodiments, these lymphocytes exhibit detectable stimulation without the need for prior non-antigen specific expansion. However, primary lymphocytes also elicit detectable antigen-specific responses when first stimulated non-specifically in vitro. Thus, in some embodiments, lymphocytes are stimulated to proliferate in vitro in a non-antigen specific manner, prior to use in an antigen presentation assay. Lymphocytes can also be stimulated in an antigen-specific manner prior to use in an antigen presentation assay. In some embodiments, cells are stimulated to proliferate by a library (e.g., prior to use in an antigen presentation assay that employs the library). Expanding cells in vitro provides greater numbers of cells for use in assays. Primary T cells can be stimulated to expand, e.g., by exposure to a polyclonal T cell mitogen, such as phytohemagglutinin or concanavalin, by treatment with antibodies that stimulate proliferation, or by treatment with particles coated with the antibodies. In some embodiments, T cells are expanded by treatment with anti-CD2, anti-CD3, and anti-CD28 antibodies. In some embodiments, T cells are expanded by treatment with interleukin-2. In some embodiments, lymphocytes are thawed from frozen storage and expanded (e.g., stimulated to proliferate, e.g., in a non-antigen specific manner or in an antigen-specific manner) prior to contacting with antigen presenting cells. In some embodiments, lymphocytes are thawed from frozen storage and are not expanded prior to contacting with antigen presenting cells. In some embodiments, lymphocytes are freshly isolated and expanded (e.g., stimulated to proliferate, e.g., in a non-antigen specific manner or in an antigen-specific manner) prior to contacting with antigen presenting cells.

### Antigen Presentation Assays

In antigen presentation assays, T cells are cultured with antigen presenting cells prepared according to the methods described above, under conditions that permit T cell recognition of peptides presented by MHC molecules on the antigen presenting cells. In some embodiments, T cells are incubated with antigen presenting cells at 37° C. for between 12-48 hours (e.g., for 24 hours). In some embodiments, T cells are incubated with antigen presenting cells at 37° C. for 3, 4, 5, 6, 7, or 8 days. Numbers of antigen presenting cells and T cells can be varied. In some embodiments, the ratio of T cells to antigen presenting cells in a given assay is 1:10, 1:5, 1:2, 1:1, 2:1, 5:1, 10:1, 20:1, 25:1, 30:1, 32:1, 35:1 or 40:1. In some embodiments, antigen presenting cells are provided in an array (e.g., in a 96-well plate), wherein cells in each location of the array have been contacted with sets of library cells, each set including a different polypeptide of interest. In certain embodiments, each location in the array includes 1×10³-1×10⁶ antigen presenting cells, and the cells are contacted with 1×10³-1×10⁶ T cells.

After T cells have been incubated with antigen presenting cells, cultures are assayed for activation. Lymphocyte activation can be detected by any means known in the art, e.g., T cell proliferation, phosphorylation or dephosphorylation of a receptor, calcium flux, cytoskeletal rearrangement, increased or decreased expression and/or secretion of immune mediators such as cytokines or soluble mediators, increased or decreased expression of one or more cell surface markers. In some embodiments, culture supernatants are harvested and assayed for increased and/or decreased expression and/or secretion of one or more polypeptides associated with activation, e.g., a cytokine, soluble mediator, cell surface marker, or other immune mediator. In some embodiments, the one or more cytokines are selected from TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANK L), MIP3-alpha, and fractalkine. In some embodiments, the one or more soluble mediators are selected from granzyme A, granzyme B, sFas, sFasL, perforin, and granulysin. In some embodiments, the one or more cell surface markers are selected from CD107a, CD107b, CD25, CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), and KLRG1. Cytokine secretion in culture supernatants can be detected, e.g., by ELISA, bead array, e.g., with a Luminex© analyzer. Cytokine production can also be assayed by RT-PCR of mRNA isolated from the T cells, or by ELISPOT analysis of cytokines released by the T cells. In some embodiments, proliferation of T cells in the cultures is determined (e.g., by detecting ³H thymidine incorporation). In some embodiments, target cell lysis is determined (e.g., by detecting T cell dependent lysis of antigen presenting cells labeled with Na₂⁵¹CrO₄). Target cell lysis assays are typically performed with CD8⁺ T cells. Protocols for these detection methods are known. See, e.g., Current Protocols In Immunology, John E. Coligan et al. (eds), Wiley and Sons, New York, N.Y., 2007. One of skill in the art understands that appropriate controls are used in these detection methods, e.g., to adjust for non-antigen specific background activation, to confirm the presenting capacity of antigen presenting cells, and to confirm the viability of lymphocytes.

In some embodiments, antigen presenting cells and lymphocytes used in the method are from the same individual. In some embodiments, antigen presenting cells and lymphocytes used in the method are from different individuals.

In some embodiments, antigen presentation assays are repeated using lymphocytes from the same individual that have undergone one or more previous rounds of exposure to antigen presenting cells, e.g., to enhance detection of responses, or to enhance weak initial responses. In some embodiments, antigen presentation assays are repeated using antigen presenting cells from the same individual that have undergone one or more previous rounds of exposure to a library, e.g., to enhance detection of responses, or to enhance weak initial responses. In some embodiments, antigen presentation assays are repeated using lymphocytes from the same individual that have undergone one or more previous rounds of exposure to antigen presenting cells, and antigen presenting cells from the same individual that have undergone one or more previous rounds of exposure to a library, e.g., to enhance detection of responses, or to enhance weak initial responses. In some embodiments, antigen presentation assays are repeated using antigen presenting cells and lymphocytes from different individuals, e.g., to identify antigens recognized by multiple individuals, or compare reactivities that differ between individuals.

### Methods of Identifying Tumor Antigens

One advantage of methods described herein is their ability to identify clinically relevant human antigens. Humans that have cancer may have lymphocytes that specifically recognize tumor antigens, which are the product of an adaptive immune response arising from prior exposure. In some embodiments, these cells are present at a higher frequency than cells from an individual who does not have cancer, and/or the cells are readily reactivated when re-exposed to the proper antigenic stimulus (e.g., the cells are “memory” cells). Thus, humans that have or have had cancer are particularly useful donors of cells for identifying antigens in vitro. The individual may be one who has recovered from cancer. In some embodiments, the individual has been recently diagnosed with cancer (e.g., the individual was diagnosed less than one year, three months, two months, one month, or two weeks, prior to isolation of lymphocytes and/or antigen presenting cells from the individual). In some embodiments, the individual was first diagnosed with cancer more than three months, six months, or one year prior to isolation of lymphocytes and/or antigen presenting cells.

In some embodiments, lymphocytes are screened against antigen presenting cells that have been contacted with a library of cells whose members express or carry polypeptides of interest, and the lymphocytes are from an individual who has not been diagnosed with cancer. In some embodiments, such lymphocytes are used to determine background (i.e., non-antigen-specific) reactivities. In some embodiments, such lymphocytes are used to identify antigens, reactivity to which exists in non-cancer individuals.

Cells from multiple donors (e.g., multiple subjects who have cancer) can be collected and assayed in methods described herein. In some embodiments, cells from multiple donors are assayed in order to determine if a given tumor antigen is reactive in a broad portion of the population, or to identify multiple tumor antigens that can be later combined to produce an immunogenic composition that will be effective in a broad portion of the population.

Antigen presentation assays are useful in the context of both infectious and non-infectious diseases. The methods described herein are applicable to any context in which a rapid evaluation of human cellular immunity is beneficial. In some embodiments, antigenic reactivity to polypeptides that are differentially expressed by neoplastic cells (e.g., tumor cells) is evaluated. Sets of nucleic acids differentially expressed by neoplastic cells have been identified using established techniques such as subtractive hybridization. Methods described herein can be used to identify antigens that were functional in a subject in which an anti-tumor immune response occurred. In other embodiments, methods are used to evaluate whether a subject has lymphocytes that react to a tumor antigen or set of tumor antigens.

In some embodiments, antigen presentation assays are used to examine reactivity to autoantigens in cells of an individual, e.g., an individual predisposed to, or suffering from, an autoimmune condition. Such methods can be used to provide diagnostic or prognostic indicators of the individual's disease state, or to identify autoantigens. For these assays, in some embodiments, libraries that include an array of human polypeptides are prepared. In some embodiments, libraries that include polypeptides from infectious agents which are suspected of eliciting cross-reactive responses to autoantigens are prepared. For examples of antigens from infectious agents thought to elicit cross-reactive autoimmune responses, see Barzilai et al., Curr Opin Rheumatol., 19(6):636-43, 2007; Ayada et al., Ann N Y Acad Sci., 1108:594-602, 2007; Drouin et al., Mol Immunol., 45(1):180-9, 2008; and Bach, J Autoimmun., 25 Suppl:74-80, 2005.

As discussed, the present disclosure includes methods in which polypeptides of interest are included in a library (e.g., expressed in library cells or carried in or on particles or beads). After members of the library are internalized by antigen presenting cells, the polypeptides of interest are proteolytically processed within the antigen presenting cells, and peptide fragments of the polypeptides are presented on MHC molecules expressed in the antigen presenting cells. The identity of the polypeptide that stimulates a human lymphocyte in an assay described herein can be determined from examination of the set of library cells that were provided to the antigen presenting cells that produced the stimulation. In some embodiments, it is useful to map the epitope within the polypeptide that is bound by MHC molecules to produce the observed stimulation. This epitope, or the longer polypeptide from which it is derived (both of which are referred to as an “antigen” herein) can form the basis for an immunogenic composition, or for an antigenic stimulus in future antigen presentation assays.

Methods for identifying peptides bound by MHC molecules are known. In some embodiments, epitopes are identified by generating deletion mutants of the polypeptide of interest and testing these for the ability to stimulate lymphocytes. Deletions that lose the ability to stimulate lymphocytes, when processed and presented by antigen presenting cells, have lost the peptide epitope. In some embodiments, epitopes are identified by synthesizing peptides corresponding to portions of the polypeptide of interest and testing the peptides for the ability to stimulate lymphocytes (e.g., in antigen presentation assays in which antigen presenting cells are pulsed with the peptides). Other methods for identifying MHC bound peptides involve lysis of the antigen presenting cells that include the antigenic peptide, affinity purification of the MHC molecules from cell lysates, and subsequent elution and analysis of peptides from the MHC (Falk, K. et al. Nature 351:290, 1991, and U.S. Pat. No. 5,989,565).

In other embodiments, it is useful to identify the clonal T cell receptors that have been expanded in response to the antigen. Clonal T cell receptors are identified by DNA sequencing of the T cell receptor repertoire (Howie et al, 2015 Sci Trans Med 7:301). By identifying TCR specificity and function, TCRs can be transfected into other cell types and used in functional studies or for novel immunotherapies. In other embodiments, it is useful to identify and isolate T cells responsive to a tumor antigen in a subject. The isolated T cells can be expanded ex vivo and administered to a subject for cancer therapy or prophylaxis.

### Methods of Identifying an Immune Response in a Subject

The disclosure provides methods of identifying one or more immune responses of a subject. In some embodiments, one or more immune responses of a subject are determined by a) providing a library described herein that includes a panel of tumor antigens (e.g., known tumor antigens, tumor antigens described herein, or tumor antigens, potential tumor antigens, and/or other polypeptides of interest identified using a method described herein); b) contacting the library with antigen presenting cells from the subject; c) contacting the antigen presenting cells with lymphocytes from the subject; and d) determining whether one or more lymphocytes are stimulated by, inhibited and/or suppressed by, activated by, or non-responsive to one or more tumor antigens presented by one or more antigen presenting cells. In some embodiments, the library includes about 1, 3, 5, 10, 15, 20, 25, 30, 40, 50, 60, 70, 80, 90, 100, or more tumor antigens.

In some embodiments, a subject is (i) a cancer subject who has not received a cancer therapy; (ii) a cancer subject who has not responded and/or is not responding and/or has responded negatively, clinically to a cancer therapy; or (iii) a subject who has not been diagnosed with a cancer.

In some embodiments, lymphocyte stimulation, non-stimulation, inhibition and/or suppression, activation, and/or non-responsiveness is determined by assessing levels of one or more expressed or secreted cytokines or other immune mediators described herein. In some embodiments, levels of one or more expressed or secreted cytokines that is at least 20%, 40%, 60%, 80%, 100%, 120%, 140%, 160%, 180%, 200% or more, higher than a control level indicates lymphocyte stimulation. In some embodiments, a level of one or more expressed or secreted cytokines that is at least 1, 2, 3, 4 or 5 standard deviations greater than the mean of a control level indicates lymphocyte stimulation. In some embodiments, a level of one or more expressed or secreted cytokines that is at least 1, 2, 3, 4 or 5 median absolute deviations (MADs) greater than a median response level to a control indicates lymphocyte stimulation. In some embodiments, a control is a negative control, for example, a clone expressing Neon Green (NG). In some embodiments, a level of one or more expressed or secreted cytokines that is at least 20%, 40%, 60%, 80%, 100%, 120%, 140%, 160%, 180%, 200% or more, lower than a control level indicates lymphocyte inhibition and/or suppression. In some embodiments, a level of one or more expressed or secreted cytokines that is at least 1, 2, 3, 4 or 5 standard deviations lower than the mean of a control level indicates lymphocyte inhibition and/or suppression. In some embodiments, a level of one or more expressed or secreted cytokines that is at least 1, 2, 3, 4 or 5 median absolute deviations (MADs) lower than a median response level to a control indicates lymphocyte inhibition and/or suppression. In some embodiments, a control is a negative control, for example, a clone expressing Neon Green (NG). In some embodiments, levels of one or more expressed or secreted cytokines that is at least 20%, 40%, 60%, 80%, 100%, 120%, 140%, 160%, 180%, 200% or more, higher or lower than a control level indicates lymphocyte activation. In some embodiments, a level of one or more expressed or secreted cytokines that is at least 1, 2, 3, 4 or 5 standard deviations greater or lower than the mean of a control level indicates lymphocyte activation. In some embodiments, a level of one or more expressed or secreted cytokines that is at least 1, 2, 3, 4 or 5 median absolute deviations (MADs) greater or lower than a median response level to a control indicates lymphocyte activation. In some embodiments, a control is a negative control, for example, a clone expressing Neon Green (NG). In some embodiments, a level of one or more expressed or secreted cytokines that is within about 20%, 15%, 10%, 5%, or less, of a control level indicates lymphocyte non-responsiveness or non-stimulation. In some embodiments, a level of one or more expressed or secreted cytokines that is less than 1 or 2 standard deviations higher or lower than the mean of a control level indicates lymphocyte non-responsiveness or non-stimulation. In some embodiments, a level of one or more expressed or secreted cytokines that is less than 1 or 2 median absolute deviations (MADs) higher or lower than a median response level to a control indicates lymphocyte non-responsiveness or non-stimulation. In some embodiments, a subject response profile can include a quantification, identification, and/or representation of a panel of different cytokines (e.g., 1, 2, 3, 4, 5, 6, 7, 8, 9, 10, 12, 14, 16, 18, 20, or more cytokines) and of the total number of tumor antigens (e.g., of all or a portion of different tumor antigens from the library) that stimulate, do not stimulate, inhibit and/or suppress, activate, or have no or minimal effect on production, expression or secretion of each member of the panel of cytokines.

### Methods of Identifying and Selecting Stimulatory and Inhibitory Tumor Antigens

In general, immune responses can be usefully defined in terms of their integrated, functional end-effects. Dhabar et al. (2014) have proposed that immune responses can be categorized as being immunoprotective, immunopathological, and immunoregulatory/inhibitory. While these categories provide useful constructs with which to organize ideas, an overall in vivo immune response is likely to consist of several types of responses with varying amounts of dominance from each category. Immunoprotective or beneficial responses are defined as responses that promote efficient wound healing, eliminate infections and cancer, and mediate vaccine-induced immunological memory. These responses are associated with cytokines and mediators such as IFN-gamma, IL-12, IL-2, Granzyme B, CD107, etc. Immunopathological or deleterious responses are defined as those that are directed against self (autoimmune disease like multiple sclerosis, arthritis, lupus) or innocuous antigens (asthma, allergies) and responses involving chronic, non-resolving inflammation. These responses can also be associated with molecules that are implicated in immunoprotective responses, but also include immune mediators such as TNF-alpha, IL-10, IL-13, IL-17, IL-4, IgE, histamine, etc. Immunoregulatory responses are defined as those that involve immune cells and factors that regulate (mostly down-regulate) the function of other immune cells. Recent studies suggest that there is an arm of the immune system that functions to inhibit immune responses. For example, regulatory CD4⁺CD25+FoxP3⁺ T cells, IL-10, and TGF-beta, among others have been shown to have immunoregulatory/inhibitory functions. The physiological function of these factors is to keep pro-inflammatory, allergic, and autoimmune responses in check, but they may also suppress anti-tumor immunity and be indicative of negative prognosis for cancer. In the context of tumors, the expression of co-stimulatory molecules often decreases, and the expression of co-inhibitory ligands increases. MHC molecules are often down-regulated on tumor cells, favoring their escape. The tumor micro-environment, including stromal cells, tumor associated immune cells, and other cell types, produce many inhibitory factors, such as, IL-10, TGF-β, and IDO. Inhibitory immune cells, including T regs, Tr1 cells, immature DCs (iDCs), pDCs, and MDSC can be found in the tumor microenvironment. (Y Li UT GSBS Thesis 2016). Examples of mediators and their immune effects are shown in Table 2.

TABLE 2
Immune Mediators

| | | | Beneficial Outcomes | | | Deleterious Outcomes | | |
|---|---|---|---|---|---|---|---|---|
| Cytokine | Function | Secreted by | Cancer | ID | Al | Cancer | ID | Al |
| TRAIL | Induces apoptosis of | Most cells | X | X | ? | X | ? | ? |
| | tumor cells, induces | | | | | | | |
| | immune suppressor | | | | | | | |
| | cells | | | | | | | |
| IFN- | Critical for innate | T cells, NK | X | X | ? | X | ? | X |
| gamma | and adaptive | cells, NKT | | | | | | |
| | immunity to | cells | | | | | | |
| | pathogens, inhibits | | | | | | | |
| | viral replication, | | | | | | | |
| | increases MHC Class | | | | | | | |
| | I expression | | | | | | | |
| IL-12 | Th1 differentiation; | DCs, macro- | X | X | ? | X | ? | X |
| | stimulates T cell | phages, | | | | | | |
| | growth, induces | neutron- | | | | | | |
| | IFN-gamma/TNF- | phils | | | | | | |
| | alpha secretion | | | | | | | |
| | from T cells, | | | | | | | |
| | enhances CTLs | | | | | | | |
| IL-2 | T cell proliferation, | T cells, APCs | X | X | X | ? | ? | ? |
| | differentiation into | | | | | | | |
| | effector and | | | | | | | |
| | memory T cells and | | | | | | | |
| | regulatory T cells | | | | | | | |
| TNF- | Induces fevers, | Macro- | X | X | ? | X | ? | X |
| alpha | apoptosis, | phages, | | | | | | |
| | inflammation, | APCs | | | | | | |
| | inhibits viral | | | | | | | |
| | replication | | | | | | | |
| MIP-1 | Chemotactic/pro- | Macro- | X | X | ? | ? | ? | X |
| alpha | inflammatory | phages, DCs, | | | | | | |
| | effects, activates | T cells | | | | | | |
| | granulocytes, | | | | | | | |
| | induces secretion of | | | | | | | |
| | IL-1/IL6/TNF-alpha | | | | | | | |
| MIP-1 | Chemotactic/pro- | Macro- | X | X | ? | ? | ? | X |
| beta | inflammatory | phages, DCs, | | | | | | |
| | effects, activates | T cells | | | | | | |
| | granulocytes, | | | | | | | |
| | induces secretion of | | | | | | | |
| | IL-1/IL6/TNF-alpha | | | | | | | |
| CXCL9 | T cell | APCs | X | X | ? | X | ? | X |
| | chemoattractant, | | | | | | | |
| | induced by IFN- | | | | | | | |
| | gamma | | | | | | | |
| CXCL10 | Chemoattractant for | APCs | X | X | ? | ? | ? | X |
| | T cells, | | | | | | | |
| | macrophages, NK | | | | | | | |
| | and DCs, promotes | | | | | | | |
| | T cell adhesion to | | | | | | | |
| | endothelial cells | | | | | | | |
| MCP-1 | Recruits monocytes, | most cells | X | X | ? | X | ? | X |
| | memory T cells and | | | | | | | |
| | DCS | | | | | | | |
| RANTES | Recruits T cells, | T cells | X | X | ? | ? | ? | X |
| | eosinophils, | | | | | | | |
| | basophils, induces | | | | | | | |
| | proliferation/activation | | | | | | | |
| | of NK cells, T cell | | | | | | | |
| | activation marker | | | | | | | |
| CXCL11 | Chemoattractant for | APCs | X | X | ? | ? | ? | X |
| | activated T cells | | | | | | | |
| IL-3 | Stimulates | T cells, APCs | X | X | ? | ? | ? | ? |
| | proliferation of | | | | | | | |
| | myeloid cells, | | | | | | | |
| | induces growth of T | | | | | | | |
| | cells | | | | | | | |
| IL-17 | Produced by Th17 | T cells | X | X | ? | X | ? | X |
| \| | cells, induces | | | | | | | |
| | production of IL6, | | | | | | | |
| | GCSF, GMCSF, IL1b, | | | | | | | |
| | TGF-beta, TNF- | | | | | | | |
| | alpha, chemokines | | | | | | | |
| IL-18 | Pro-inflammatory, | Macro- | X | X | ? | X | ? | X |
| | induces cell- | phages | | | | | | |
| | mediated immunity, | | | | | | | |
| | production of IFN- | | | | | | | |
| | gamma | | | | | | | |
| IL-21 | Induces | CD4 T cells | X | X | X | X | ? | ? |
| | proliferation, | | | | | | | |
| | upregulated in | | | | | | | |
| | Th2/Th17 TFh | | | | | | | |
| IL-22 | Cell-mediated | NK cells, T | X | X | ? | X | ? | X |
| | immunity, pro- | cells | | | | | | |
| | inflammatory | | | | | | | |
| IL-23 | Pro-inflammatory | APCs | X | X | ? | X | ? | X |
| IL-24 | Controls survival | Monocytes | X | X | ? | ? | ? | X |
| | and proliferation | macro- | | | | | | |
| | | phages, Th2 | | | | | | |
| | | cells | | | | | | |
| IL-27 | Induces | APCs, T cells | X | X | X | X | ? | X |
| | differentiation of T | | | | | | | |
| | cells, upregulates IL- | | | | | | | |
| | 10, can be pro-or | | | | | | | |
| | anti-inflammatory; | | | | | | | |
| | promotes Th1/Tr1, | | | | | | | |
| | inhibits Th2/Th17/ | | | | | | | |
| | regulatory T cells | | | | | | | |
| IL-32 | Pro-inflammatory, | T cells, NK | X | X | ? | X | ? | X |
| | increases secretion | cells | | | | | | |
| | of inflammatory | | | | | | | |
| | cytokines and | | | | | | | |
| | chemokines | | | | | | | |
| CSF | Induces myeloid | APCs | X | X | X | ? | ? | ? |
| | cells to proliferate | | | | | | | |
| | and differentiate | | | | | | | |
| GM-CSF | Promotes | T cells, | X | X | ? | ? | ? | X |
| | macrophage and | macro- | | | | | | |
| | Eosinophil | phages | | | | | | |
| | proliferation and | | | | | | | |
| | maturation, growth | | | | | | | |
| | factor | | | | | | | |
| TRANCE | Helps DC | T cells | ? | X | ? | X | ? | ? |
| | maturation/survival, | | | | | | | |
| | T cell activation | | | | | | | |
| | marker, anti- | | | | | | | |
| | apoptotic, | | | | | | | |
| | stimulates | | | | | | | |
| | osteoclast activity | | | | | | | |
| MIP-3 | Chemotactic for T | | X | X | ? | ? | ? | X |
| alpha | cells, DCs | | | | | | | |
| fractalkine | Chemotactic for T | Endothelial | X | X | ? | ? | ? | X |
| | cells and monocytes | cells | | | | | | |
| IL-4 | Stimulates B cells, | Th2 cells, | ? | X | ? | X | X | X |
| | Th2 proliferation, | basophils | | | | | | |
| | plasma cell | | | | | | | |
| | differentiation, IgE, | | | | | | | |
| | upregulates MHC | | | | | | | |
| | Class II expression, | | | | | | | |
| | decreases IFN- | | | | | | | |
| | gamma production | | | | | | | |
| IL-10 | Downregulates Th1 | Monocytes | X | ? | X | X | X | X |
| | cytokines/MHC | Th2 cells, | | | | | | |
| | Class II | regulatory T | | | | | | |
| | expression/Co- | cells | | | | | | |
| | stimulatory | | | | | | | |
| | molecule expression | | | | | | | |
| IL-5 | Stimulates B cells, Ig | Th2 cells, | ? | X | ? | X | X | X |
| | secretion, | mast cells | | | | | | |
| | eosinophil | | | | | | | |
| | activation | | | | | | | |
| IL-13 | Similar to IL4, | Th2 cells, NK | ? | X | ? | X | X | X |
| | induces IgE | cells, mast | | | | | | |
| | production, Th2 | cells, | | | | | | |
| | cytokine | eosinophils, | | | | | | |
| | | basophils | | | | | | |
| TGF-beta | Inhibits T cell | regulatory T | ? | ? | X | X | X | ? |
| | proliferation, | cells | | | | | | |
| | activity, function; | | | | | | | |
| | blocks effects of | | | | | | | |
| | pro-inflammatory | | | | | | | |
| | cytokines | | | | | | | |
| IL-1 beta | Induces fevers, pro- | Macro- | X | X | ? | X | ? | X |
| | inflammatory | phages | | | | | | |
| IL-6 | Pro-inflammatory, | T cells, | ? | X | ? | X | X | X |
| | drives osteoclast | macro- | | | | | | |
| | formation, drives | phages | | | | | | |
| | Th17 | | | | | | | |
| IL-8 | Recruits neutrophils | Macro- | ? | X | ? | X | ? | X |
| | to site of infection | phages, | | | | | | |
| | | epithelial | | | | | | |
| | | cells | | | | | | |
| IL-31 | Cell-mediated | Th2 cells, | X | X | ? | X | ? | X |
| | immunity, pro- | macro- | | | | | | |
| | inflammatory | phages, DCs | | | | | | |
| IL-15 | T cell proliferation | T cells, NK | X | X | X | ? | ? | ? |
| | and survival | cells | | | | | | |
| IL-9 | Th2 proliferation, | T cells, | ? | ? | X | X | X | ? |
| | cytokine secretion | neutrophils, | | | | | | |
| | | mast cells | | | | | | |

ID = Infectious disease
IA = Autoimmune disease

In some embodiments, a stimulatory antigen is a tumor antigen (e.g., a tumor antigen described herein) that stimulates one or more lymphocyte responses that are beneficial to the subject. In some embodiments, a stimulatory antigen is a tumor antigen (e.g., a tumor antigen described herein) that inhibits and/or suppresses one or more lymphocyte responses that are deleterious or non-beneficial to the subject. Examples of immune responses that may lead to beneficial anti-tumor responses (e.g., that may enhance immune control of a tumor) include but are not limited to 1) cytotoxic CD8⁺ T cells which can effectively kill cancer cells and release the mediators perform and/or granzymes to drive tumor cell death; and 2) CD4⁺ Th1 T cells which play an important role in host defense and can secrete IL-2, IFN-gamma and TNF-alpha. These are induced by IL-12, IL-2, and IFN gamma among other cytokines.

In some embodiments, an inhibitory antigen is a tumor antigen (e.g., a tumor antigen described herein) that stimulates one or more lymphocyte responses that are deleterious or non-beneficial to the subject. In some embodiments, an inhibitory antigen is a tumor antigen (e.g., a tumor antigen described herein) that inhibits and/or suppresses one or more lymphocyte responses that are beneficial to the subject. Examples of immune responses that may lead to deleterious or non-beneficial anti-tumor responses (e.g., that may impair or reduce control of a tumor) include but are not limited to 1) T regulatory cells which are a population of T cells that can suppress an immune response and secrete immunosuppressive cytokines such as TGF-beta and IL-10 and express the molecules CD25 and FoxP3; and 2) Th2 cells which target responses against allergens but are not productive against cancer. These are induced by increased IL-4 and IL-10 and can secrete IL-4, IL-5, IL-6, IL-9 and IL-13.

The disclosure provides methods and systems for identifying and selecting tumor antigens, e.g., stimulatory and/or inhibitory antigens. In some embodiments, one or more selected antigen is a stimulatory antigen. A stimulatory antigen may be selected based on the measured immune response to the antigen using a method of the disclosure. A stimulatory antigen may be selected if the antigen produces an immune response that stimulates the expression and/or release of one or more of any cytokine associated with a beneficial response, as shown for example, in Table 2. In some embodiments, the cytokine comprises one or more of IL-2, IFN-gamma and TNF-alpha. A stimulatory antigen may be selected if the antigen produces an immune response that inhibits the expression and/or release of one or more of any of the cytokines associated with a deleterious response, as shown for example, in Table 2. In some embodiments, the cytokine comprises one or more of TGF-beta and IL-10.

In some embodiments, a stimulatory antigen is selected if the level of one or more of the expressed or secreted cytokines associated with a beneficial response is at least 20%, 40%, 60%, 80%, 100%, 120%, 140%, 160%, 180%, 200% or more, higher than a control level indicates lymphocyte stimulation. In some embodiments, a stimulatory antigen is selected if the level of one or more of the expressed or secreted cytokines associated with a beneficial response is at least 1, 2, 3, 4 or 5 standard deviations greater than the mean of a control level indicates lymphocyte stimulation. In some embodiments, a stimulatory antigen is selected if the level of one or more of the expressed or secreted cytokines associated with a beneficial response is at least 1, 2, 3, 4 or 5 median absolute deviations (MADs) greater than a median response level to a control indicates lymphocyte stimulation. In some embodiments, a control is a negative control, for example, a clone expressing Neon Green (NG).

In some embodiments, a stimulatory antigen is selected if the level of one or more of the expressed or secreted cytokines associated with a deleterious response is at least 20%, 40%, 60%, 80%, 100%, 120%, 140%, 160%, 180%, 200% or more, lower than a control level indicates lymphocyte inhibition and/or suppression. In some embodiments, a stimulatory antigen is selected if the level of one or more of the expressed or secreted cytokines associated with a deleterious response is at least 1, 2, 3, 4 or 5 standard deviations lower than the mean of a control level indicates lymphocyte inhibition and/or suppression. In some embodiments, a stimulatory antigen is selected if the level of one or more of the expressed or secreted cytokines associated with a deleterious response is at least 1, 2, 3, 4 or 5 median absolute deviations (MADs) lower than a median response level to a control that indicates lymphocyte inhibition and/or suppression. In some embodiments, a control is a negative control, for example, a clone expressing Neon Green (NG).

In some embodiments, one or more selected antigen is an inhibitory antigen. An inhibitory antigen may be de-selected based on a measured immune response to the antigen using a method of the disclosure. An inhibitory antigen may be selected if the antigen produces an immune response that stimulates the expression and/or release of one or more cytokines associated with a deleterious response, as shown for example, in Table 2. In some embodiments, the cytokine comprises one or more of TGF-beta and IL-10. An inhibitory antigen may be selected if the antigen produces an immune response that inhibits the expression and/or release of one or more of any cytokine associated with a beneficial response, as shown for example, in Table 2. In some embodiments, the cytokine comprises one or more of IL-2, IFN-gamma and TNF-alpha.

In some embodiments, an inhibitory antigen is selected if the level of one or more of the expressed or secreted cytokines associated with a deleterious response is at least 20%, 40%, 60%, 80%, 100%, 120%, 140%, 160%, 180%, 200% or more, higher than a control level indicates lymphocyte stimulation. In some embodiments, an inhibitory antigen is selected if the level of one or more of the expressed or secreted cytokines associated with a deleterious response is at least 1, 2, 3, 4 or 5 standard deviations greater than the mean of a control level indicates lymphocyte stimulation. In some embodiments, an inhibitory antigen is selected if the level of one or more of the expressed or secreted cytokines associated with a deleterious response is at least 1, 2, 3, 4 or 5 median absolute deviations (MADs) greater than a median response level to a control that indicates lymphocyte stimulation. In some embodiments, a control is a negative control, for example, a clone expressing Neon Green (NG).

In some embodiments, an inhibitory antigen is selected if the level of one or more of the expressed or secreted cytokines associated with a beneficial response is at least 20%, 40%, 60%, 80%, 100%, 120%, 140%, 160%, 180%, 200% or more, lower than a control level that indicates lymphocyte inhibition and/or suppression. In some embodiments, an inhibitory antigen is selected if the level of one or more of the expressed or secreted cytokines associated with a beneficial response is at least 1, 2, 3, 4 or 5 standard deviations lower than the mean of a control level that indicates lymphocyte inhibition and/or suppression. In some embodiments, an inhibitory antigen is selected if the level of one or more of the expressed or secreted cytokines associated with a beneficial response is at least 1, 2, 3, 4 or 5 median absolute deviations (MADs) lower than a median response level to a control that indicates lymphocyte inhibition and/or suppression. In some embodiments, a control is a negative control, for example, a clone expressing Neon Green (NG).

### Production of Tumor Antigens

A tumor antigen suitable for use in any method or composition of the disclosure may be produced by any available means, such as recombinantly or synthetically (see, e.g., Jaradat Amino Acids 50:39-68 (2018); Behrendt et al., J. Pept. Sci. 22:4-27 (2016)). For example, a tumor antigen may be recombinantly produced by utilizing a host cell system engineered to express a tumor antigen-encoding nucleic acid. Alternatively or additionally, a tumor antigen may be produced by activating endogenous genes. Alternatively or additionally, a tumor antigen may be partially or fully prepared by chemical synthesis.

Where proteins are recombinantly produced, any expression system can be used. To give but a few examples, known expression systems include, for example, E. coli, egg, baculovirus, plant, yeast, or mammalian cells.

In some embodiments, recombinant tumor antigen suitable for the present invention are produced in mammalian cells. Non-limiting examples of mammalian cells that may be used in accordance with the present invention include BALB/c mouse myeloma line (NSO/|, ECACC No: 85110503); human retinoblasts (PER.C6, CruCell, Leiden, The Netherlands); monkey kidney CV1 line transformed by SV40 (COS-7, ATCC CRL 1651); human embryonic kidney line (HEK293 or 293 cells subcloned for growth in suspension culture, Graham et al., J. Gen Virol., 36:59,1977); human fibrosarcoma cell line (e.g., HT1080); baby hamster kidney cells (BHK21, ATCC CCL 10); Chinese hamster ovary cells+/−DHFR (CHO, Urlaub and Chasm, Proc. Natl. Acad. Sci. USA, 77:4216, 1980); mouse sertoli cells (TM4, Mather, Biol. Reprod., 23:243-251, 1980); monkey kidney cells (CV1 ATCC CCL 70); African green monkey kidney cells (VERO-76, ATCC CRL-1 587); human cervical carcinoma cells (HeLa, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci., 383:44-68, 1982); MRC 5 cells; FS4 cells; and a human hepatoma line (Hep G2).

In some embodiments, the present disclosure provides recombinant tumor antigen produced from human cells. In some embodiments, the present disclosure provides recombinant tumor antigen produced from CHO cells or HT1080 cells.

Typically, cells that are engineered to express a recombinant tumor antigen may comprise a transgene that encodes a recombinant tumor antigen described herein. It should be appreciated that the nucleic acids encoding recombinant tumor antigen may contain regulatory sequences, gene control sequences, promoters, non-coding sequences and/or other appropriate sequences for expressing the recombinant tumor antigen. Typically, the coding region is operably linked with one or more of these nucleic acid components.

The coding region of a transgene may include one or more silent mutations to optimize codon usage for a particular cell type. For example, the codons of a tumor antigen transgene may be optimized for expression in a vertebrate cell. In some embodiments, the codons of a tumor antigen transgene may be optimized for expression in a mammalian cell. In some embodiments, the codons of a tumor antigen transgene may be optimized for expression in a human cell.

### Immunogenic Compositions and Uses Thereof

The present disclosure provides compositions (e.g., immunogenic compositions) that include a tumor antigen or tumor antigens identified or selected by methods described herein, nucleic acids encoding the tumor antigens, and methods of using the compositions. In some embodiments, a composition includes tumor antigens that are peptides 8-40 amino acids, 8-60 amino acids, 8-100, 8-150, or 8-200 amino acids in length (e.g., MHC binding peptides, e.g., peptides 23-29, 24-28, 25-27, 8-30, 8-29, 8-28, 8-27, 8-26, 8-25, 8-24, 8-23, 8-22, 8-21, 8-20, 8-15, 8-12 amino acids in length). In some embodiments, a composition includes one or more tumor antigens that are about 70%, 75%, 80%, 85%, 90%, 95%, 96%, 97%, 98%, 99% or 100% of the length of the full-length polypeptides. In some embodiments, a composition includes one or more tumor antigens that are truncated by about 1, 2, 3, 4, 5, 10, 15, 20, 25, 30, 35, 40, 45, 50, or more amino acids, relative to the full-length polypeptides. The compositions can include tumor antigens that are, or that comprise, MHC class I-binding peptides, MHC class II-binding peptides, or both MHC class I and MHC class II-binding peptides. Compositions can include a single tumor antigen, or multiple tumor antigens. In some embodiments, a composition includes a set of two, three, four, five, six, seven, eight, nine, ten, or more tumor antigens. In some embodiments, a composition includes ten, fifteen, twenty, twenty-five, thirty, or more tumor antigens. In some embodiments, the tumor antigens or peptides are provided as one or more fusion proteins. In some embodiments, a composition comprises nucleic acids encoding the tumor antigens or peptides. In some embodiments, the nucleic acids encoding the tumor antigens or peptides are provided as one or more fusion constructs. In some embodiments, an immunogenic composition includes a tumor antigen linked to a carrier protein. Examples of carrier proteins include, e.g., toxins and toxoids (chemical or genetic), which may or may not be mutant, such as anthrax toxin, PA and DNI (PharmAthene, Inc.), diphtheria toxoid (Massachusetts State Biological Labs; Serum Institute of India, Ltd.) or CRM 197, tetanus toxin, tetanus toxoid (Massachusetts State Biological Labs; Serum Institute of India, Ltd.), tetanus toxin fragment Z, exotoxin A or mutants of exotoxin A of Pseudomonas aeruginosa, bacterial flagellin, pneumolysin, an outer membrane protein of Neisseria meningitidis (strain available from the ATCC (American Type Culture Collection, Manassas, Va.)), Pseudomonas aeruginosa Hcp1 protein, E. coli heat labile enterotoxin, shiga-like toxin, human LTB protein, a protein extract from whole bacterial cells, and any other protein that can be cross-linked by a linker. Other useful carrier proteins include high density lipoprotein (HDL), bovine serum albumin (BSA), P40, and chicken riboflavin. Many carrier proteins are commercially available (e.g., from Sigma Aldrich).

The disclosure also provides nucleic acids encoding the tumor antigens. The nucleic acids can be used to produce expression vectors, e.g., for recombinant production of the tumor antigens, or for nucleic acid-based administration in vivo (e.g., DNA vaccination).

In some embodiments, an immunogenic composition may be suitable for administration to a human patient, and vaccine preparation may conform to USFDA guidelines. In some embodiments, an immunogenic composition is suitable for administration to a non-human animal. In some embodiments, an immunogenic composition is substantially free of either endotoxins or exotoxins. Endotoxins include pyrogens, such as lipopolysaccharide (LPS) molecules. An immunogenic composition may also be substantially free of inactive protein fragments. In some embodiments, an immunogenic composition has lower levels of pyrogens than industrial water, tap water, or distilled water. Other components of the immunogenic composition may be purified using methods known in the art, such as ion-exchange chromatography, ultrafiltration, or distillation. In other embodiments, the pyrogens may be inactivated or destroyed prior to administration to a patient. Raw materials for immunogenic compositions, such as water, buffers, salts and other chemicals may also be screened and depyrogenated. All materials in a immunogenic composition may be sterile, and each lot of the composition may be tested for sterility. Thus, in certain embodiments the endotoxin levels in the immunogenic composition fall below the levels set by the USFDA, for example 0.2 endotoxin (EU)/kg of product for an intrathecal injectable composition; 5 EU/kg of product for a non-intrathecal injectable composition, and 0.25-0.5 EU/ml for sterile water.

In some embodiments, an immunogenic composition (e.g., a vaccine and/or a vaccine formulation) comprising a polypeptide contains less than 5%, 2%, 1%, 0.5%, 0.2%, 0.1% of other, undesired unpolypeptides, relative to the amount of desired polypeptides. In some embodiments, an immunogenic composition contains less than 5%, less than 2%, less than 1%, less than 0.5%, less than 0.2%, or less than 0.1% DNA and/or RNA.

Immunogenic compositions can be prepared as formulations suitable for route of administration. Formulations suitable for parenteral administration, such as, for example, by intraarticular (in the joints), intravenous, intramuscular, intradermal, intraperitoneal, intranasal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampoules and vials. Injection solutions and suspensions can be prepared from sterile powders, granules, and tablets of the kind previously described.

Adjuvants

Immunogenic compositions described herein may include an adjuvant. Adjuvants can be used as vaccine delivery systems and/or for their immunostimulatory properties. Vaccine delivery systems are often particulate formulations, e.g., emulsions, microparticles, immune-stimulating complexes (ISCOMs), which may be, for example, particles and/or matrices, and liposomes. Immunostimulatory adjuvants include ISCOMS or may be derived from pathogens and can represent pathogen associated molecular patterns (PAMP), e.g., lipopolysaccharides (LPS), monophosphoryl lipid (MPL), or CpG-containing DNA, which activate cells of the innate immune system. An exemplary adjuvant is Poly-ICLC (Hiltonol, Oncovir Inc).

Adjuvants may also be classified as organic and inorganic. Inorganic adjuvants include aluminum salts such as aluminum phosphate, amorphous aluminum hydroxyphosphate sulfate, and aluminum hydroxide, which are commonly used in human vaccines. Organic adjuvants comprise organic molecules including macromolecules. An example of an organic adjuvant is cholera toxin.

Adjuvants may also be classified by the response they induce, and adjuvants can activate more than one type of immunostimulatory response. In some embodiments, the adjuvant induces the activation of CD4+ T cells. The adjuvant may induce activation of TH1 cells and/or activation of TH17 cells and/or activation of TH2 cells. Alternately, the adjuvant may induce activation of TH1 cells and/or TH17 cells but not activation of TH2 cells, or vice versa. In some embodiments, the adjuvant induces activation of CD8+ T cells. In further embodiments, the adjuvant may induce activation of Natural Killer T (NKT) cells. In some embodiments, the adjuvant induces the activation of TH1 cells or TH17 cells or TH2 cells. In other embodiments, the adjuvant induces the activation of B cells. In yet other embodiments, the adjuvant induces the activation of APCs. These categories are not mutually exclusive; in some cases, an adjuvant activates more than one type of cell.

In certain embodiments, an adjuvant stimulates an immune response by increasing the numbers or activity of APCs such as dendritic cells. In certain embodiments, an adjuvant promotes the maturation of APCs such as dendritic cells. In some embodiments, the adjuvant is or comprises a saponin. In some embodiments, a saponin adjuvant is immunostimulatory. Typically, a saponin is a triterpene glycoside, such as those isolated from the bark of the Quillaja saponaria tree. A saponin extract from a biological source can be further fractionated (e.g., by chromatography) to isolate the portions of the extract with the best adjuvant activity and with acceptable toxicity. Typical fractions of extract from Quillaja saponaria tree used as adjuvants are known as fractions A and C. An exemplary saponin adjuvant is QS-21 (fraction C), which is available from Antigenics. QS-21 is an oligosaccharide-conjugated small molecule. Optionally, QS-21 may be admixed with a lipid such as 3D-MPL or cholesterol.

A particular form of saponins that may be used in vaccine formulations described herein is immunostimulating complexes (ISCOMs). ISCOMs are an art-recognized class of adjuvants, that generally comprise Quillaja saponin fractions and lipids (e.g., cholesterol and phospholipids such as phosphatidyl choline). In certain embodiments, an ISCOM is assembled together with a polypeptide or nucleic acid of interest. However, different saponin fractions may be used in different ratios. In addition, the different saponin fractions may either exist together in the same particles or have substantially only one fraction per particle (such that the indicated ratio of fractions A and C are generated by mixing together particles with the different fractions). In this context, “substantially” refers to less than 20%, 15%, 10%, 5%, 4%, 3%, 2% or even 1%. Such adjuvants may comprise fraction A and fraction C mixed into a ratio of 70-95 A:30-5 C, such as 70 A:30 C to 75 A:25 C; 75 A:25 C to 80 A:20 C; 80 A:20 C to 85 A:15 C; 85 A:15 C to 90 A:10 C; 90 A:10 C to 95 A:5 C; or 95 A:5 C to 99 A:1 C. ISCOMatrix, produced by CSL, and AbISCO 100 and 300, produced by Isconova, are ISCOM matrices comprising saponin, cholesterol and phospholipid (lipids from cell membranes), which form cage-like structures typically 40-50 nm in diameter. Posintro, produced by Nordic Vaccines, is an ISCOM matrix where the immunogen is bound to the particle by a multitude of different mechanisms, e.g., electrostatic interaction by charge modification, incorporation of chelating groups, or direct binding.

In some embodiments, the adjuvant is Matrix-M2 (MM2). In some embodiments, the Matrix-M2 adjuvant comprises saponin fractions purified from Quillaja saponaria (soapbark tree) bark, phosphatidylcholine and cholesterol. In some embodiments, the adjuvant is diluted in normal saline, for example 0.9% saline.

In some embodiments, the adjuvant is a TLR ligand. TLRs are proteins that may be found on leukocyte membranes, and recognize foreign antigens (including microbial antigens). An exemplary TLR ligand is IC-31, which is available from Intercell. IC-31 comprises an anti-microbial peptide, KLK, and an immunostimulatory oligodeoxynucleotide, ODN1a. IC-31 has TLR9 agonist activity. Another example is CpG-containing DNA. Different varieties of CpG-containing DNA are available from Prizer (Coley): VaxImmune is CpG 7909 (a (CpG)-containing oligodeoxy-nucleotide), and Actilon is CpG 10101 (a (CpG)-containing oligodeoxy-nucleotide).

In some embodiments, the adjuvant is a nanoemulsion. One exemplary nanoemulsion adjuvant is Nanostat Vaccine, produced by Nanobio. This nanoemulsion is a high-energy, oil-in-water emulsion. This nanoemulsion typically has a size of 150-400 nanometers, and includes surfactants to provide stability. More information about Nanostat can be found in U.S. Pat. Nos. 6,015,832, 6,506,803, 6,559,189, 6,635,676, and 7,314,624.

In some embodiments, an adjuvant includes a cytokine. In some embodiments, the cytokine is an interleukin such as ILL-1, IL-6, IL-12, IL-17 and IL-23. In some embodiments, the cytokine is granulocyte-macrophage colony-stimulating factor (GM-CSF). The adjuvant may include cytokine as a purified polypeptide. Alternatively, the adjuvant may include nucleic acids encoding the cytokine.

Adjuvants may be covalently bound to antigens (e.g., the polypeptides described above). In some embodiments, the adjuvant may be a protein which induces inflammatory responses through activation of APCs. In some embodiments, one or more of these proteins can be recombinantly fused with an antigen of choice, such that the resultant fusion molecule promotes dendritic cell maturation, activates dendritic cells to produce cytokines and chemokines, and ultimately, enhances presentation of the antigen to T cells and initiation of T cell responses (see Wu et al., Cancer Res 2005; 65(11), pp 4947-4954). Other exemplary adjuvants that may be covalently bound to antigens comprise polysaccharides, synthetic peptides, lipopeptides, and nucleic acids.

The adjuvant can be used alone or in combination of two or more kinds. Adjuvants may be directly conjugated to antigens. Adjuvants may be administered in therapeutically effective amounts, for example, an amount that produces the desired effect (e.g., immunostimulatory effect) for which it is administered. Adjuvants may also be combined to increase the magnitude of the immune response to the antigen. Typically, the same adjuvant or mixture of adjuvants is present in each dose of an immunogenic composition (e.g., a vaccine and/or a vaccine formulation). Optionally, however, an adjuvant may be administered with a first dose of an immunogenic composition and not with subsequent doses (e.g., additional dose(s) or maintenance dose(s)). Alternatively, a strong adjuvant may be administered with the first dose of an immunogenic composition and a weaker adjuvant or lower dose of the strong adjuvant may be administered with subsequent doses. The adjuvant can be administered before the administration of the antigen, concurrent with the administration of the antigen or after the administration of the antigen to a subject (sometimes within 1, 2, 6, or 12 hours, and sometimes within 1, 2, or 5 days). Certain adjuvants are appropriate for human patients, non-human animals, or both.

Additional Components

In addition to the antigens and the adjuvants described above, an immunogenic composition, e.g., a vaccine, a vaccine formulation and/or a pharmaceutical composition, may include one or more additional components.

In certain embodiments, an immunogenic composition may include one or more stabilizers such as sugars (such as sucrose, glucose, or fructose), phosphate (such as sodium phosphate dibasic, potassium phosphate monobasic, dibasic potassium phosphate, or monosodium phosphate), glutamate (such as monosodium L-glutamate), gelatin (such as processed gelatin, hydrolyzed gelatin, or porcine gelatin), amino acids (such as arginine, asparagine, histidine, L-histidine, alanine, valine, leucine, isoleucine, serine, threonine, lysine, phenylalanine, tyrosine, and the alkyl esters thereof), inosine, or sodium borate.

In certain embodiments, an immunogenic composition includes one or more buffers such as a mixture of sodium bicarbonate and ascorbic acid. In some embodiments, an immunogenic composition may be administered in saline (e.g., 0.9% saline), such as phosphate buffered saline (PBS), or distilled water.

In certain embodiments, an immunogenic composition includes one or more surfactants such as polysorbate 80 (Tween 80), Polyethylene glycol tert-octylphenyl ether t-Octylphenoxypolyethoxyethanol 4-(1,1,3,3-Tetramethylbutyl)phenyl-polyethylene glycol (TRITON X-100); Polyoxyethylenesorbitan monolaurate Polyethylene glycol sorbitan monolaurate (TWEEN 20); and 4-(1,1,3,3-Tetramethylbutyl)phenol polymer with formaldehyde and oxirane (TYLOXAPOL). A surfactant can be ionic or nonionic.

In certain embodiments, an immunogenic composition includes one or more salts such as sodium chloride, ammonium chloride, calcium chloride, or potassium chloride.

In certain embodiments, a preservative is included in an immunogenic composition. In other embodiments, no preservative is used. A preservative is most often used in multi-dose vaccine vials, and is less often needed in single-dose vaccine vials. In certain embodiments, the preservative is 2-phenoxyethanol, methyl and propyl parabens, benzyl alcohol, and/or sorbic acid.

In certain embodiments, an immunogenic composition is a controlled-release formulation.

Dosing Regimens

In some embodiments, an immunogenic composition is administered to a subject according to a dosing regimen or dosing schedule. The amount of antigen in each immunogenic composition dose (e.g., a vaccine, vaccine formulation and/or pharmaceutical composition) is selected to be a therapeutically effective amount, which induces a prophylactic or therapeutic response, as described above, in either a single dose or over multiple doses. Preferably, a dose is without significant adverse side effects in typical immunogenic compositions. Such amount will vary depending upon which specific antigen is employed. Generally, it is expected that a single dose will comprise about 100 to about 1500 μg total peptide. In some embodiments, a total volume of a single dose is 0.5 mL to 1.0 mL. In some embodiments, a single dose will comprise more than one antigen, for example, 2, 3, 4, 5 or more.

In some embodiments, a dosing regimen comprises an initial dose of an immunogenic composition and at least one additional dose of the immunogenic composition. In some embodiments, after an initial dose is administered, an additional dose is administered about 3 weeks following the initial dose. In some embodiments, an additional dose is administered about 6 weeks following the initial dose. In some embodiments, an additional dose is administered about 12 weeks following the initial dose. In some embodiments, and an additional dose is administered about 24 weeks following the initial dose.

In some embodiments, the dosing regimen comprises administration of different immunogenic compositions, e.g., 2, 3, 4, 5, 6, 7, 8, or more different immunogenic compositions comprising antigens. In some embodiments, each dose comprises administering different immunogenic compositions, e.g., in succession. In some embodiments, each dose comprises administering the same set of different immunogenic compostions. For example, a dosing regimen can include an initial dose of 2, 3, 4, 5, 6, 7, 8, or more different immunogenic compositions, and at least one additional dose of the 2, 3, 4, 5, 6, 7, 8, or more different immunogenic compositions. In some embodiments, an immunogenic composition comprises one antigen. In some embodiments, an immunogenic composition comprises 2, 3, 4, 5, 6, 7, 8, 9, 10, or more antigens.

In some embodiments, a dosing regimen can include an initial dose of 2, 3, 4, 5, 6, 7, 8, or more different immunogenic compositions (e.g., where each immunogenic composition can separately include 2, 3, 4, 5, 6, 7, 8, 9, 10, or more antigens), and at least one additional dose of the 2, 3, 4, 5, 6, 7, 8, or more different immunogenic compositions. For example, a dosing regimen can include an initial dose of 4 different immunogenic compositions, where each immunogenic composition comprises 1, 2, 3, 4 or 5 different antigens. In some embodiments, such dosing regimen further includes at least 1 (e.g., at least 2, 3, 4, 5, 6, or more) additional doses of the 4 different immunogenic compositions. In some embodiments, a second dose is administered about 1, 2, 3, 4, or 5 weeks after the initial dose; a third dose is administered about 1, 2, 3, 4, or 5 weeks after the second dose; a fourth dose is administered about 2, 4, 6, 8, 10, 12, 14, 16, or 18 weeks after the third dose; and a fifth dose is administered about 2, 4, 6, 8, 10, 12, 14, 16, or 18 weeks after the fourth dose.

### Uses

In some embodiments, tumor antigens are used in diagnostic assays. For these assays, compositions including the tumor antigens can be provided in kits, e.g., for detecting antibody reactivity, or cellular reactivity, in a sample from an individual.

In some embodiments, tumor antigen compositions are used to induce an immune response in a subject. In some embodiments, the subject is a human. In some embodiments, the subject is a non-human animal. The tumor antigen compositions can be used to raise antibodies (e.g., in a non-human animal, such as a mouse, rat, hamster, or goat), e.g., for use in diagnostic assays, and for therapeutic applications. For an example of a therapeutic use, a tumor antigen discovered by a method described herein may be a potent T cell and/or B cell antigen. Preparations of antibodies may be produced by immunizing a subject with the tumor antigen and isolating antiserum from the subject. Methods for eliciting high titers of high affinity, antigen-specific antibodies, and for isolating the tumor antigen-specific antibodies from antisera, are known in the art. In some embodiments, the tumor antigen compositions are used to raise monoclonal antibodies, e.g., human monoclonal antibodies.

In some embodiments, a tumor antigen composition is used to induce an immune response in a human subject to provide a therapeutic response. In some embodiments, a tumor antigen composition is used to induce an immune response in a human subject that redirects an undesirable immune response. In some embodiments, a tumor antigen composition elicits an immune response that causes the subject to have a positive clinical response described herein, e.g., as compared to a subject who has not been administered the tumor antigen composition. In some embodiments, a tumor antigen composition elicits an immune response that causes the subject to have an improved clinical response, e.g., as compared to a subject who has not been administered the tumor antigen composition. In some embodiments, a tumor antigen composition is used to induce an immune response in a human subject for palliative effect. The response can be complete or partial therapy.

In some embodiments, a tumor antigen composition is used to induce an immune response in a human subject to provide a prophylactic response. The response can be complete or partial protection.

In some embodiments, immunogenicity of a tumor antigen is evaluated in vivo. In some embodiments, humoral responses to a tumor antigen are evaluated (e.g., by detecting antibody titers to the administered tumor antigen). In some embodiments, cellular immune responses to a tumor antigen are evaluated, e.g., by detecting the frequency of antigen-specific cells in a sample from the subject (e.g., by staining T cells from the subject with MHC/peptide tetramers containing the antigenic peptide, to detect antigen-specific T cells, or by detecting antigen-specific cells using an antigen presentation assay such as an assay described herein). In some embodiments, the ability of a tumor antigen or antigens to elicit protective or therapeutic immunity is evaluated in an animal model. In some embodiments, the ability of a tumor antigen or antigens to stimulate or to suppress and/or inhibit immunity is evaluated in an animal model.

### Cancer and Cancer Therapy

The present disclosure provides methods and systems related to subjects having or diagnosed with cancer, such as a tumor. In some embodiments, a tumor is or comprises a hematologic malignancy, including but not limited to, acute lymphoblastic leukemia, acute myeloid leukemia, chronic lymphocytic leukemia, chronic myelogenous leukemia, hairy cell leukemia, AIDS-related lymphoma, Hodgkin lymphoma, non-Hodgkin lymphoma, Langerhans cell histiocytosis, multiple myeloma, or myeloproliferative neoplasms.

In some embodiments, a tumor is or comprises a solid tumor, including but not limited to breast carcinoma, a squamous cell carcinoma, a colon cancer, a head and neck cancer, ovarian cancer, a lung cancer, mesothelioma, a genitourinary cancer, a rectal cancer, a gastric cancer, or an esophageal cancer.

In some particular embodiments, a tumor is or comprises an advanced tumor, and/or a refractory tumor. In some embodiments, a tumor is characterized as advanced when certain pathologies are observed in a tumor (e.g., in a tissue sample, such as a biopsy sample, obtained from a tumor) and/or when cancer patients with such tumors are typically considered not to be candidates for conventional chemotherapy. In some embodiments, pathologies characterizing tumors as advanced can include tumor size, altered expression of genetic markers, invasion of adjacent organs and/or lymph nodes by tumor cells. In some embodiments, a tumor is characterized as refractory when patients having such a tumor are resistant to one or more known therapeutic modalities (e.g., one or more conventional chemotherapy regimens) and/or when a particular patient has demonstrated resistance (e.g., lack of responsiveness) to one or more such known therapeutic modalities.

In some embodiments, the present disclosure provides methods and systems related to cancer therapy. The present disclosure is not limited to any specific cancer therapy, and any known or developed cancer therapy is encompassed by the present disclosure. Known cancer therapies include, e.g., administration of chemotherapeutic agents, radiation therapy, surgical excision, chemotherapy following surgical excision of tumor, adjuvant therapy, localized hypothermia or hyperthermia, anti-tumor antibodies, and anti-angiogenic agents. In some embodiments, cancer and/or adjuvant therapy includes a TLR agonist (e.g., CpG, Poly I:C, etc., see, e.g., Wittig et al., Crit. Rev. Oncol. Hematol. 94:31-44 (2015); Huen et al., Curr. Opin. Oncol. 26:237-44 (2014); Kaczanowska et al., J. Leukoc. Biol. 93:847-863 (2013)), a STING agonist (see, e.g., US20160362441; US20140329889; Fu et al., Sci. Transl. Med. 7:283ra52 (2015); and WO2014189805), a non-specific stimulus of innate immunity, and/or dendritic cells, or administration of GM-CSF, Interleukin-12, Interleukin-7, Flt-3, or other cytokines. In some embodiments, the cancer therapy is or comprises oncolytic virus therapy, e.g., talimogene leherparepvec. (see, e.g., Fukuhara et al., Cancer Sci. 107:1373-1379 (2016)). In some embodiments, the cancer therapy is or comprises bi-specific antibody therapy (e.g., Choi et al., 2011 Expert Opin Biol Ther; Huehls et al., 2015, Immunol and Cell Biol). In some embodiments, the cancer therapy is or comprises cellular therapy such as chimeric antigen receptor T (CAR-T) cells, TCR-transduced T cells, dendritic cells, tumor infiltrating lymphocytes (TIL), or natural killer (NK) cells (e.g., as reviewed in Sharpe and Mount, 2015, Dis Model Mech 8:337-50).

Anti-tumor antibody therapies (i.e., therapeutic regimens that involve administration of one or more anti-tumor antibody agents) are rapidly becoming the standard of care for treatment of many tumors. Antibody agents have been designed or selected to bind to tumor antigens, particularly those expressed on tumor cell surfaces. Various review articles have been published that describe useful anti-tumor antibody agents (see, for example, Adler et al., Hematol. Oncol. Clin. North Am. 26:447-81 (2012); Li et al., Drug Discov. Ther. 7:178-84 (2013); Scott et al., Cancer Immun. 12:14 (2012); and Sliwkowski et al., Science 341:1192-1198 (2013)). The below Table 3 presents a non-comprehensive list of certain human antigens targeted by known, available antibody agents, and notes certain cancer indications for which the antibody agents have been proposed to be useful:

TABLE 3

| | Antibody (commercial or | |
|---|---|---|
| Human Antigen | scientific name) | Cancer indication |
| CD2 | Siplizumab | Non-Hodgkin's Lymphoma |
| CD3 | UCHT1 | Peripheral or Cutaneous T-cell Lymphoma |
| CD4 | HuMax-CD4 | |
| CD19 | SAR3419, MEDI-551 | Diffuse Large B-cell Lymphoma |
| CD19 and CD3 or | Bispecific antibodies such as | Non-Hodgkin's Lymphoma |
| CD22 | Blinatumomab, DT2219ARL | |
| CD20 | Rituximab, Veltuzumab, | B cell malignancies (Non-Hodgkin's |
| | Tositumomab, Ofatumumab, | lymphoma, Chronic lymphocytic leukemia) |
| | Ibritumomab, Obinutuzumab, | |
| CD22 (SIGLEC2) | Inotuzumab, tetraxetan,CAT- | Chemotherapy-resistant hairy cell leukemia, |
| | 8015, DCDT2980S, Bectumomab | Hodgkin's lymphoma |
| CD30 | Brentuximab vedotin | |
| CD33 | Gemtuzumab ozogamicin | Acute myeloid leukemia |
| | (Mylotarg) | |
| CD37 | TRU-016 | Chronic lymphocytic leukemia |
| CD38 | Daratumumab | Multiple myeloma, hematological tumors |
| CD40 | Lucatumumab | Non-Hodgkin's lymphoma |
| CD52 | Alemtuzumab (Campath) | Chronic lymphocytic leukemia |
| CD56 (NCAM1) | Loniotuzumab | Small Cell Lung Cancer |
| CD66e (CEA) | Labetuzumab | Breast, colon and lung tumors |
| CD70 | SGN-75 | Non-Hodgkin's lymphoma |
| CD74 | Milatuzumab | Non-Hodgkin's lymphoma |
| CD138 (SYND1) | BT062 | Multiple Myeloma |
| CD152 (CTLA-4) | Ipilimumab | Metastatic melanoma |
| CD221 (IGF1R) | AVE1642, IMC-A12, MK-0646, | Glioma, lung, breast, head and neck, |
| | R150, CP 751871 | prostate and thyroid cancer |
| CD254 (RANKL) | Denosumab | Breast and prostate carcinoma |
| CD261 (TRAILR1) | Mapatumumab | |
| CD262 (TRAILR2) | HGS-ETR2, CS-1008 | Colon, lung and pancreas tumors and |
| | | haematological malignancies |
| CD326 (Epcam) | Edrecolomab, 17-1A, IGN101, | Colon and rectal cancer, malignant ascites, |
| | Catumaxomab, Adecatumumab | epithelial tumors (breast, colon, lung) |
| CD309 (VEGFR2) | IM-2C6, CDP791 | Epithelium-derived solid tumors |
| CD319 (SLAMF7) | HuLuc63 | Multiple myeloma |
| CD340 (HER2) | Trastuzumab, Pertuzumab, Ado- | Breast cancer |
| | trastuzumab emtansine | |
| CAIX (CA9) | cG250 | Renal cell carcinoma |
| EGFR (c-erbB) | Cetuximab, Panitumumab, | Solid tumors including glioma, lung, breast, |
| | nimotuzumab and 806 | colon, and head and neck tumors |
| EPHA3 (HEK) | KB004, IIIA4 | Lung, kidney and colon tumors, melanoma, |
| | | glioma and haematological malignancies |
| Episialin | Epitumomab | Epithelial ovarian tumors |
| FAP | Sibrotuzumab and F19 | Colon, breast, lung, pancreas, and head and |
| | | neck tumors |
| HLA-DR beta | Apolizumab | Chronic lymphocytic leukemia, non- |
| | | Hodkin's lymphoma |
| FOLR-1 | Farletuzumab | Ovarian tumors |
| 5T4 | Anatumomab | Non-small cell lung cancer |
| GD3/GD2 | 3F8, ch14.18, KW-2871 | Neuroectodermal and epithelial tumors |
| gpA33 | huA33 | Colorectal carcinoma |
| GPNMB | Glembatumumab | Breast cancer |
| HER3 (ERBB3) | MM-121 | Breast, colon, lung, ovarian, and prostate |
| | | tumors |
| Integrin αVβ3 | Etaracizumab | Tumor vasculature |
| Integrin α5β1 | Volociximab | Tumor vasculature |
| Lewis-Y antigen | hu3S193, IgN311 | Breast, colon, lung and prostate tumors |
| MET (HGFR) | AMG 102, METMAB, SCH90015 | Breast, ovary and lung tumors |
| Mucin-1/CanAg | Pemtumomab, oregovomab, | Breast, colon, lung and ovarian tumors |
| | Cantuzumab | |
| PSMA | ADC, J591 | Prostate Cancer |
| Phosphatidylserine | Bavituximab | Solid tumors |
| TAG-72 | Minretumomab | Breast, colon and lung tumors |
| Tenascin | 81C6 | Glioma, breast and prostate tumours |
| VEGF | Bevacizumab | Tumor vasculature |
| PD-L1 | Avelumab | Non-small cell lung cancer, MCC |
| CD274 | Durvalumab | Non-small cell lung cancer |
| IDO enzyme | IDO inhibitors | Multiple |

In some embodiments, a cancer therapy is or comprises immune checkpoint blockade therapy (see, e.g., Martin-Liberal et al., Cancer Treat. Rev. 54:74-86 (2017); Menon et al., Cancers (Basel) 8:106 (2016)), or immune suppression blockade therapy. Certain cancer cells thrive by taking advantage of immune checkpoint pathways as a major mechanism of immune resistance, particularly with respect to T cells that are specific for tumor antigens. For example, certain cancer cells may overexpress one or more immune checkpoint proteins responsible for inhibiting a cytotoxic T cell response. Thus, immune checkpoint blockade therapy may be administered to overcome the inhibitory signals and permit and/or augment an immune attack against cancer cells. Immune checkpoint blockade therapy may facilitate immune cell responses against cancer cells by decreasing, inhibiting, or abrogating signaling by negative immune response regulators (e.g., CTLA-4). In some embodiments, a cancer therapy or may stimulate or enhance signaling of positive regulators of immune response (e.g., CD28).

Examples of immune checkpoint blockade and immune suppression blockade therapy include agents targeting one or more of A2AR, B7-H4, BTLA, CTLA-4, CD28, CD40, CD137, GITR, IDO, KIR, LAG-3, PD-1, PD-L1, OX40, TIM-3, and VISTA. Specific examples of immune checkpoint blockade agents include the following monoclonal antibodies: ipilimumab (targets CTLA-4); tremelimumab (targets CTLA-4); atezolizumab (targets PD-L1); pembrolizumab (targets PD-1); nivolumab (targets PD-1); avelumab; durvalumab; and cemiplimab.

Specific examples of immune suppression blockade agents include: Vista (B7-H5, v-domain Ig suppressor of T cell activation) inhibitors; Lag-3 (lymphocyte-activation gene 3, CD223) inhibitors; IDO (indolemamine-pyrrole-2,3,-dioxygenase-1,2) inhibitors; KIR receptor family (killer cell immunoglobulin-like receptor) inhibitors; CD47 inhibitors; and Tigit (T cell immunoreceptor with Ig and ITIM domain) inhibitors.

In some embodiments, a cancer therapy is or comprises immune activation therapy. Specific examples of immune activators include: CD40 agonists; GITR (glucocorticoid-induced TNF-R-related protein, CD357) agonists; OX40 (CD134) agonists; 4-1BB (CD137) agonists; ICOS (inducible T cell stimulator); CD278 agonists; IL-2 (interleukin 2) agonists; and interferon agonists.

In some embodiments, cancer therapy is or comprises a combination of one or more immune checkpoint blockade agents, immune suppression blockade agents, and/or immune activators, or a combination of one or more immune checkpoint blockade agents, immune suppression blockade agents, and/or immune activators, and other cancer therapies.

As discussed herein, in some embodiments, the present disclosure provides methods and systems related to subjects who do not respond and/or have not responded; or respond and/or have responded (e.g., clinically responsive, e.g., clinically positively responsive or clinically negatively responsive) to a cancer therapy. In some embodiments, subjects respond and/or have responded positively clinically to a cancer therapy. In some embodiments, subjects respond and/or have responded negatively clinically to a cancer therapy. In some embodiments, subjects do not respond and/or have not responded (e.g., clinically non-responsive) to a cancer therapy.

Whether a subject responds positively, responds negatively, and/or fails to respond to a cancer therapy can be measured and/or characterized according to particular criteria. In certain embodiments, such criteria can include clinical criteria and/or objective criteria. In certain embodiments, techniques for assessing response can include, but are not limited to, clinical examination, positron emission tomography, chest X-ray, CT scan, MRI, ultrasound, endoscopy, laparoscopy, presence or level of a particular marker in a sample, cytology, and/or histology. A positive response, a negative response, and/or no response, of a tumor to a therapy can be assessed by ones skilled in the art using a variety of established techniques for assessing such response, including, for example, for determining one or more of tumor burden, tumor size, tumor stage, etc. Methods and guidelines for assessing response to treatment are discussed in Therasse et al., J. Natl. Cancer Inst., 2000, 92(3):205-216; and Seymour et al., Lancet Oncol., 2017, 18:e143-52.

In some embodiments, a responsive subject exhibits a decrease in tumor burden, tumor size, and/or tumor stage upon administration of a cancer therapy. In some embodiments, a non-responsive subject does not exhibit a decrease in tumor burden, tumor size, or tumor stage upon administration of a cancer therapy. In some embodiments, a non-responsive subject exhibits an increase in tumor burden, tumor size, or tumor stage upon administration of a cancer therapy.

In some embodiments, a cancer subject is identified and/or selected for administration of a cancer therapy as described herein. In some embodiments, the cancer therapy is administered to the subject. In some embodiments, upon administration of the cancer therapy, the subject exhibits a positive clinical response to the cancer therapy, e.g., exhibits an improvement based on one or more clinical and/or objective criteria (e.g., exhibits a decrease in tumor burden, tumor size, and/or tumor stage). In some embodiments, the clinical response is more positive than a clinical response to the cancer therapy administered to a cancer subject who is identified (using a method described herein) as a cancer subject who should not initiate, and/or should modify (e.g., reduce and/or combine with one or more other modalities), and/or should discontinue the cancer therapy, and/or should initiate an alternative cancer therapy.

Methods described herein can include preparing and/or providing a report, such as in electronic, web-based, or paper form. The report can include one or more outputs from a method described herein, e.g., a set of stimulatory and/or inhibitory antigens described herein. In some embodiments, a report is generated, such as in paper or electronic form, which identifies the presence or absence of one or more tumor antigens (e.g., one or more stimulatory and/or inhibitory and/or suppressive tumor antigens, or tumor antigens to which lymphocytes are not responsive, described herein) for a cancer patient, and optionally, a recommended course of cancer therapy. In some embodiments, the report includes an identifier for the cancer patient. In one embodiment, the report is in web-based form.

In some embodiments, additionally or alternatively, a report includes information on prognosis, resistance, or potential or suggested therapeutic options. The report can include information on the likely effectiveness of a therapeutic option, the acceptability of a therapeutic option, or the advisability of applying the therapeutic option to a cancer patient, e.g., identified in the report. For example, the report can include information, or a recommendation, on the administration of a cancer therapy, e.g., the administration of a pre-selected dosage or in a pre-selected treatment regimen, e.g., in combination with one or more alternative cancer therapies, to the patient. The report can be delivered, e.g., to an entity described herein, within 7, 14, 21, 30, or 45 days from performing a method described herein. In some embodiments, the report is a personalized cancer treatment report.

In some embodiments, a report is generated to memorialize each time a cancer subject is tested using a method described herein. The cancer subject can be reevaluated at intervals, such as every month, every two months, every six months or every year, or more or less frequently, to monitor the subject for responsiveness to a cancer therapy and/or for an improvement in one or more cancer symptoms, e.g., described herein. In some embodiments, the report can record at least the treatment history of the cancer subject.

In one embodiment, the method further includes providing a report to another party. The other party can be, for example, the cancer subject, a caregiver, a physician, an oncologist, a hospital, clinic, third-party payor, insurance company or a government office.

In some embodiments, an immunogenic composition described herein (e.g., an immunogenic composition comprising one or more stimulatory antigens described herein) is administered in combination with one or more cancer therapies. Combination therapy refers to those situations in which a subject or population of subjects is simultaneously exposed to two or more therapeutic agents (e.g., an immunogenic composition and a cancer therapy). In some embodiments, the two or more therapies may be administered simultaneously (e.g., concurrently). In some embodiments, such therapies may be administered sequentially (e.g., all “doses” of a first therapeutic agent are administered prior to administration of any doses of a second therapeutic agent).

In some embodiments, “administration” of combination therapy may involve administration of one or more agents or modalities to a subject receiving the other agents or modalities in the combination. For clarity, combination therapy does not require that individual agents be administered together in a single composition (or even necessarily at the same time), although in some embodiments, two or more agents, or active moieties thereof, may be administered together in a combination composition, or even in a combination compound (e.g., as part of a single chemical complex or covalent entity).

All publications, patent applications, patents, and other references mentioned herein are incorporated by reference in their entirety. In addition, the materials, methods, and examples are illustrative only and not intended to be limiting. Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

The disclosure is further illustrated by the following examples. The examples are provided for illustrative purposes only. They are not to be construed as limiting the scope or content of the disclosure in any way.

### EXAMPLES

### Example 1. Clinical Evaluation of GEN 009

A Phase 1/2a Study to Evaluate the Safety, Tolerability, Immunogenicity, and Antitumor Activity of GEN 009 Adjuvanted Vaccine in Adult Patients with Selected Solid Tumors

### Study Phase: 1/2a

Number of Patients: Up to 99 evaluable patients.

### Study Design Overview

This first-in-human, open-label, multicenter, Phase 1/2a study of GEN 009 is conducted in adult patients with the following tumor types:
Melanoma (cutaneous)
Non-small cell lung cancer (NSCLC)
Squamous cell carcinoma of the head and neck (SCCHN) (oral, oropharyngeal, hypopharyngeal, or laryngeal)
Urothelial carcinoma (bladder, ureter, urethra, or renal pelvis)
Renal cell carcinoma (RCC) with a clear cell component (Part B only)

GEN 009 is an investigational, personalized adjuvanted vaccine that is being developed for the treatment of patients with solid tumors. A system as described above, and herein called ATLAS (Antigen Lead Acquisition System), is used to identify neoantigens in each patient's tumor that are recognized by their CD4+ and/or CD8+ T cells. ATLAS-identified neoantigens that are recognized by CD4+ and/or CD8+ T cells, and are shown to be stimulatory antigens, are incorporated into a patient's personalized vaccine in the form of synthetic long peptides (SLPs). A personalized vaccine, consisting of 4 to 20 SLPs, is generated for each patient. The SLPs are divided into 4 pools, with each pool containing 1 to 5 SLPs. The 4 pools are administered subcutaneously (SC) in each of the patient's limbs. Collectively, these pools of SLPs are the GEN 009 drug product. If fewer than 4 pools are available due to manufacturing, stability, or other issues, the patient is vaccinated with the available drug product. Each pool of GEN 009 drug product consists of 100 to 1500 μg total peptide administered with 0.45 mg poly-ICLC adjuvant (Hiltonol) per injection.

This study is conducted in 2 parts as follows:

Part A: Schedule Evaluation of GEN 009 Monotherapy in Patients with No Evidence of Disease

In Part A, the safety and immunogenicity of GEN 009 monotherapy is evaluated in patients with cutaneous melanoma, NSCLC, SCCHN, or urothelial carcinoma who have completed treatment with curative intent for their disease (eg, surgical resection, neoadjuvant and/or adjuvant chemotherapy and/or radiation therapy) and have no evidence of disease (NED) by the time of initiating vaccination with GEN 009.

A 5-dose schedule is evaluated (Schedule 1; Days 1, 22, 43, 85 [12 weeks after Day 1], and 169 [24 weeks after Day 1]).

### Patient Screening/Vaccine Manufacture

After informed consent is provided, each potential Part A patient undergoes leukapheresis for collection of peripheral blood mononuclear cells (PBMC). PBMC, along with samples of tumor and saliva (PBMCs from leukapheresis are used for SCCHN patients due to potential malignant contamination), are subjected to next-generation sequencing (NGS) and the ATLAS process to identify potential neoantigens.

Following completion of the ATLAS process, the patient is reevaluated. In order to proceed with vaccine manufacture, a sufficient number of stimulatory antigens for SLP manufacture must have been identified from the ATLAS process, and the patient must continue to meet study eligibility criteria, including NED on a radiographic disease assessment performed within 8 weeks prior to the reevaluation.

Following completion of vaccine manufacture and prior to Study Day 1 (the first dose of GEN 009), the patient is reevaluated again. A disease assessment performed within 12 weeks prior to the second reevaluation and no more than 4 weeks prior to Day 1 vaccination must show NED, and patients must continue to meet eligibility criteria, including recovery from any clinically significant toxicity from prior therapies, in order to receive GEN 009.

Any patient with a recurrence of disease during the screening period for Part A may be considered for Part B if they meet the eligibility criteria for Part B when this arm is actively enrolling.

Part B: GEN 009 in Patients with Advanced or Metastatic Solid Tumors

Part B includes patients with one of 5 tumor types (NSCLC, SCCHN, cutaneous melanoma, urothelial carcinoma or RCC) who enroll in a disease-specific expansion cohort (up to 15 response evaluable patients each). During the screening period, patients receive the tumor type-specific treatments identified below (i.e., PD-1 inhibitor monotherapy or PD-1 inhibitor in combination, per disease-specific standard of care and USPI). Following completion of this screening period therapy and for patients who continue to meet study eligibility and will continue on PD-1 inhibitor therapy, GEN 009 dosing is initiated (starting as soon as the vaccine is available and at the schedule selected in Part A) in combination with the PD-1 inhibitor:
NSCLC: pembrolizumab with chemotherapy (pemetrexed and platinum chemotherapy for non-squamous histologies; carboplatin and either paclitaxel or nab-paclitaxel for squamous NSCLC) during the screening period, followed by pembrolizumab and GEN 009 during the treatment period;
SCCHN: pembrolizumab monotherapy during the screening period, followed by pembrolizumab and GEN 009 during the treatment period;
Cutaneous melanoma: nivolumab monotherapy or nivolumab in combination with ipilimumab during the screening period, followed by nivolumab and GEN 009 during the treatment period;
Urothelial carcinoma: pembrolizumab monotherapy during the screening period, followed by pembrolizumab and GEN 009 during the treatment period;
RCC: nivolumab monotherapy or nivolumab in combination with ipilimumab during the screening period, followed by nivolumab and GEN 009 during the treatment period.

In addition, approximately 15 patients enrolled in one of the above disease-specific cohorts but whose disease progresses during the screening period therapy may be enrolled into a separate relapsed/refractory disease cohort.

Each cohort in Part B is evaluated for safety, immunogenicity, and antitumor activity.

### Patient Screening Vaccine Manufacture

After informed consent is provided, each potential Part B patient undergoes leukapheresis for collection of PBMCs. PBMCs, along with samples of tumor (obtained after the patient's most recent systemic cancer therapy, if applicable, prior to initiation of the PD-1 inhibitor, and not from a previously irradiated lesion) and saliva (PBMCs from leukapheresis will be used for SCCHN patients due to potential malignant contamination in saliva), are subjected to NGS and the ATLAS process. A baseline radiographic disease assessment (DA #1) is performed within 4 weeks prior to initiation of the PD-1 inhibitor (I chemotherapy or ipilimumab, as applicable); scans performed within this timeframe according to standard of care are acceptable.

After collection of tumor and PBMC samples and baseline radiographic assessment, patients in Part B initiate therapy consisting of nivolumab (as monotherapy or with ipilimumab for cutaneous melanoma and RCC) or pembrolizumab (as monotherapy for SCCHN and urothelial carcinoma, or with chemotherapy for NSCLC).

Repeat radiographic disease assessments is performed 6 to 10 weeks after initiation of the PD-1 inhibitor (DA #2) and within 14 days prior to Day 1 of GEN 009 dosing (DA #3); scans performed within these timeframes according to standard of care are acceptable. Patients who have adequate disease control (potentially including patients with minimal disease progression per RECIST v1.1) during these time frames and do not need alternate therapy in the opinion of the investigator and the patient, and who continue to meet study eligibility criteria are dosed with GEN 009. Patients who have progressive disease (PD) on their PD-1 inhibitor-containing regimen prior to vaccination and require alternate therapy may be allowed to continue in the study during their alternate therapy and be vaccinated at an appropriate time in their disease course in the opinion of the Investigator and the Medical Monitor, if the patient continues to meet performance and laboratory eligibility criteria. These patients (i.e., the relapsed/refractory cohort) are assessed separately for objective response rate (ORR). Patients with a complete response (CR) prior to vaccination may be dosed with GEN 009 at the discretion of the Investigator and with agreement from the Sponsor/Medical Monitor pending GEN 009 availability. These patients do not count toward the 15 response-evaluable patients in each cohort. Patients receiving ipilimumab or chemotherapy along with the PD-1 inhibitor must complete these therapies at least 14 days prior to Day 1 of GEN 009 dosing.

### Treatment Period—Parts A and B

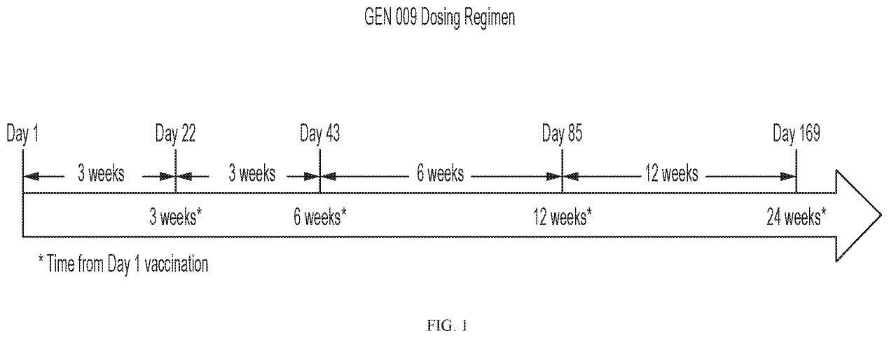

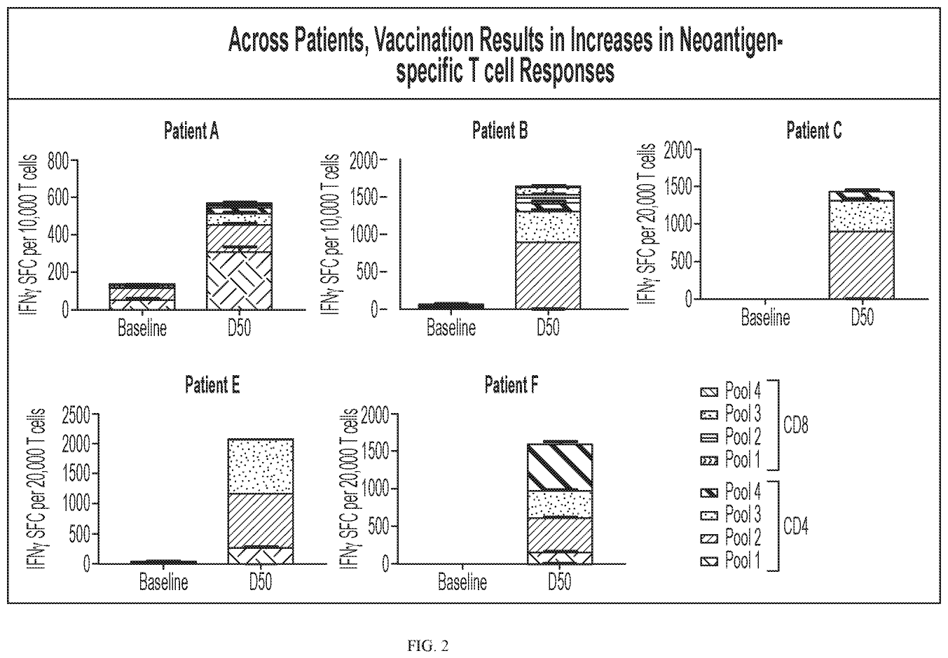

The dosing schedules are outlined in Table 4 and depicted visually in FIG. 1. A 5-dose schedule is evaluated (Schedule 1; Days 1, 22, 43, 85 [12 weeks after Day 1], and 169 [24 weeks after Day 1]).

Patients may continue to receive GEN 009 through Day 169 as long as they are tolerating treatment without recurrence (Part A) or progression of disease (Part B), and do not meet another treatment withdrawal criterion. Patients in Part B with evidence of progression may continue treatment beyond RECIST v1.1 progression if continued treatment is consistent with iRECIST principles, and if the patient and treating investigator believe that alternate treatment is not immediately necessary, and only upon Sponsor/Medical Monitor approval.

TABLE 4
Administration Schedule of GEN 009

| Schedule | Dosing Frequency |
|---|---|
| Schedule 1 | Days 1, 22, 43; |
| | boosters at Day 85 & Day 169 |

### Post Treatment Period—Parts A and B

All patients return 30 days after their last dose of GEN 009 for an end of treatment evaluation. All patients who are alive, not lost to follow-up, and/or who have not withdrawn consent are followed for safety for 1 year after their last dose of GEN 009. Data regarding subsequent therapy and response to those therapies are collected during this follow-up period. Patients who demonstrate immunogenicity at Day 366 are asked to provide an additional blood sample for immunogenicity testing at approximately Day 547 (18 months following Day 1). On-study disease assessments by imaging continue until disease recurrence (Part A), disease progression (Part B), initiation of another systemic anticancer therapy, or study closure.

### Criteria for Evaluation—Parts A and B

### Immunogenicity:

In all parts, patient blood samples are drawn to evaluate the immunogenicity of GEN 009 on Day 29, Day 50, Day 92, Day 176, Day 366 (i.e., after 1 year) and Day 547 (i.e., after 18 months if the Day 366 sample demonstrates immunogenicity). T cell responses in peripheral blood mononuclear cells (PBMCs) is assessed by interferon-gamma (IFN γ)/granzyme B (GrB) FluoroSpot assay or by IFN 7/tumor necrosis factor-alpha (TNF-α) FluoroSpot assay (mean spot-forming cells, fold change, and responder rate per SLP). CD4+ and CD8+ polyfunctional T cell responses in PBMCs is assessed by immune assays such as intracellular cytokine staining. Phenotypes of PBMC cell populations before and after vaccination are assessed by assays such as flow cytometry-based immunophenotyping panels examining regulatory T cells, activation/inhibition markers, and potentially other cell populations.

### Clinical Activity:

Patients are assessed by CT or MRI for the following clinical endpoints:

Part A: Disease-free survival (DFS). Disease assessment (radiological imaging and for patients with urothelial carcinoma who have not undergone cystectomy, urine cytology; and for patients with tumor potentially visible by cystoscopy [eg, of the urethra, bladder, ureterovesical junction], cystoscopy) will be performed during the screening period (as per study eligibility), then every 12 weeks (starting 12 weeks after Day 1) through Day 337 (i.e., 4 assessments post-Day 1), then every 26 weeks until disease recurrence, initiation of another systemic anticancer therapy, or study closure. Note: PET/CT may be used instead of CT or MRI per agreement of the Medical Monitor and Investigator for patients in Part A.

Part B: Since the GEN 009 vaccine is administered after 3 to 4 months of known active therapy, a traditional response rate and duration of response is difficult to evaluate. In general, the great majority of patients will have defined the course of their disease within those 3 to 4 months, so that any significant change in trajectory after addition of the vaccine likely represents an impact of the vaccine, noting that pseudoprogression could be responsible for a small percentage of responses. In this setting, the patient serves as their own control in an exploratory analysis of RIR, DoR, and PFS. Study-specific disease assessments (radiological imaging) are obtained during screening within 4 weeks prior to initiation of PD-1 inhibitor therapy, 6 to 10 weeks after initiation of PD-1 inhibitor therapy, and within 14 days prior to the first dose of GEN 009. Post first dose, study-specific disease assessment occurs at Day 50 (±3 days) and Day 92 (±3 days). Additionally, throughout the study, standard of care disease assessments are recorded until disease progression, initiation of another systemic anticancer therapy, or study closure. Antitumor activity is also assessed by improvement in tumor growth kinetics (i.e., increase in tumor shrinkage rate or decrease in tumor growth rate) with GEN 009 vs projected rate without GEN 009.

### Statistical Methods:

The primary categorization for data summary and analysis consists of the separate parts of the study. Within Part A, additional categories for summarization consist of all schedules studied, as well as overall for certain data presentations. For Part B, data are analyzed separately for patients with PD prior to GEN 009 dosing. Further categories for data summarization for Part B consist of data for each tumor type, data for those with relapsed/refractory disease, and overall. Select safety presentations may use an overall pool across parts for summarization, as appropriate.

All statistics are expected to be descriptive and include number of patients and number of SLPs, mean, median, standard deviation (SD), and minimum/maximum for continuous variables. Categorical variables are tabulated by number of observations and proportions. Time to event distribution is estimated using Kaplan-Meier techniques. When appropriate, the median along with CI will be provided.

For Immunogenicity Analyses: A positive cellular immune response for a given SLP is determined using statistical and/or empirical criteria. Cellular immune responses to GEN 009 are summarized for each patient by magnitude of response and/or fold change from baseline for each time point. Immune responses are summarized for each tumor type and for all patients combined.

Statistical tests such as Wilcoxon rank sum test are used to compare magnitude of response between tumor types or changes before and after vaccination when applicable.

For Clinical Activity Analyses: For Part A, DFS is summarized using Kaplan-Meier methods. For Part B, RIR is tabulated by frequency distribution, with 2-sided exact 90% CIs. Median time to response and DoR are summarized for those patients with confirmed responses, using Kaplan Meier methods. PFS and overall survival (OS) are similarly summarized. In addition, the rate of patients with PFS and OS of at least 12 months duration is presented with 2-sided 90% CIs. RIR in Part B is summarized as categorical data and by use of shift tables. Improvement in tumor growth kinetics, which is measured by comparing observed tumor growth rate with GEN 009 vs projected tumor growth rate without GEN 009 for each period from Day 1 is summarized by period for each patient. Observed tumor growth rate for each period is calculated as the average percent change in the sum of the longest diameters from earlier time points when imaging was collected; and projected tumor growth rate post Day 1 is a weighted average of observed tumor growth rates prior to Day 1, where time points closer to Day 1 are assigned with heavier weighting.

Clinical activity analyses are descriptive; statistical tests may be used as appropriate to compare changes before and after vaccination or between tumor types. Subgroup analysis of various immunologic parameters, as well as rate of response and time to event endpoints, based on demographic and baseline disease characteristics may be performed as well as exploratory analyses, as appropriate.

For Interim Analysis: For each cohort in Part B (tumor-specific and relapsed/refractory), an ongoing non-binding interim analysis is planned for the initial response evaluable patients' responses.

### Example 2. Immunogenicity of GEN 009 Vaccine

As described in Example 1, GEN-009-101 is a first-in-human phase 1/2a study testing ATLAS platform feasibility, safety, immunogenicity and clinical activity in selected solid tumors. After next-generation sequencing of patient tumors and cytokine-based ATLAS assessment using autologous T cells and APCs, up to 20 stimulatory synthetic long peptides (SLPs), corresponding to ATLAS-identified, patient-specific stimulatory antigens (neoantigens), were used in each personalized vaccine. For each patient, SLPs were divided into 4 pools, each comprising 1 to 5 SLPs. For each patient, the GEN 009 vaccine comprised the 4 pools of SLPs. GEN 009 was administered with poly-ICLC on Day 1 (week 0), Day 22 (week 3), Day 43 (week 6) with booster vaccinations on Day 85 (week 12 after Day 1 vaccination) and Day 169 (week 24 after Day 1 vaccination). PBMCs were collected for immunogenicity assessments from whole blood drawn at Day 1 vaccination (just prior to vaccination) and at Day 29, Day 92, and Day 176 (i.e., one week following vaccinations on Day 22, Day 85, and Day 169), and also via a leukapheresis procedure at initial patient screening (baseline) and at either Day 50, Day 92, or Day 176 (i.e., one week following vaccinations on Day 43, Day 85, or Day 169). Plasma was also obtained from these samples.

### Ex Vivo FluoroSpot Assay

The cellular immune response to GEN 009 was monitored by examining T cell responses using a dual-color FluoroSpot assay. The ex vivo FluoroSpot assay simultaneously detects release of interferon gamma (IFN-γ) and Granzyme B (GrB) from PBMCs, or T cell subsets enriched from PBMCs, following stimulation with peptide antigens for a duration of approximately 2 days. This method varies the traditional Enzyme-linked ImmunoSpot (ELISpot) assay by replacing the colorimetric detection with fluorescence detection, enabling quantification of individual, peptide-reactive T cells that secrete multiple analytes of interest in a high-throughput format. In general, the method detects effector T cell responses.

For each patient, complete PBMC populations, or CD4+ or CD8+ T cells enriched from PBMCs, were stimulated with overlapping peptides spanning either the unique individual SLPs or all SLPs included in each of the 4 pools of SLPs for that patient to determine the frequency of antigen-specific T cells. PBMCs or T cells enriched from PBMCs were combined with overlapping peptides spanning patient-specific SLPs in pre-conditioned, polyvinylidene difluoride membrane-bound 96-well plates, and incubated at 37° C. for 44±4 hours. Development of immune response-induced fluorescent spots was facilitated by addition of detection antibodies (anti-IFN-γ monoclonal antibody and biotinylated anti-Granzyme B monoclonal antibody) followed by anti-BAM-490 and SA-550 fluorescent antibodies.

### In Vitro Stimulated FluoroSpot Assay

The in vitro stimulated (IVS) FluoroSpot assay simultaneously detects release of interferon gamma (IFN-γ) and tumor necrosis factor alpha (TNF-α) from T cell subsets enriched from PBMCs, following in vitro stimulation (IVS) with peptide antigens for a duration of approximately 10 days in culture. The method is aimed at generating increased polyfunctional, peptide-reactive T cells over the course of the culture period. In general, the method detects memory T cell responses.

For each patient, CD4+ or CD8+ T cells enriched from PBMCs were expanded in culture for 10 days with overlapping peptides spanning the unique individual SLPs or all SLPs included in each of the 4 pools of SLPs for that patient in the presence of IL-7. On days 2, 4 and 7, half the culture media was changed and IL-2, IL-15 and IL-21 were added. On day 9, cells were rested in fresh culture media without cytokines. The expanded T cells were then combined with fresh antigen presenting cells and their respective overlapping peptides spanning patient-specific SLPs in pre-conditioned, polyvinylidene difluoride membrane-bound 96-well plates, and incubated at 37° C. for 20±4 hours. Development of immune response-induced fluorescent spots was facilitated by addition of detection antibodies (anti-IFN-γ monoclonal antibody and biotinylated anti-TNF-α monoclonal antibody) followed by anti-BAM-490 and SA-550 fluorescent antibodies.

### Results

Repeated dosing with GEN 009 was well tolerated with only mild local discomfort and no dose-limiting toxicity. ATLAS screening results show inter-patient variability in the number of stimulatory and inhibitory antigens and immune profile. Table 5 summarizes the tumor mutational burden (TMB; mutations/Mb of DNA), the number of ATLAS-identified, patient-specific antigens eliciting stimulatory or inhibitory T cell responses as measured by IFN-γ and/or TNF-α secretion, the number of patient-specific SLPs included in each vaccine, and prior therapies for each patient selected for GEN 009 vaccination. All values were generated prior to GEN 009 vaccination.

TABLE 5
Patients screened and selected for GEN 009 vaccination

| | Tumor | | TMB | Stim | Inhib | SLPs in |
|---|---|---|---|---|---|---|
| Patient | Type | Therapy | (mut/Mb) | Ags | Ags | Vaccine |
| A | SqNSCLC | Surgery, | 0.18 | 6 | 0 | 10 |
| | | Carbo, Etop | | | | |
| B | Urothelial | Surgery, | 0.9 | 16 | 4 | 8 |
| | | Mito, Cis, | | | | |
| | | Gem, Pembro | | | | |
| C | Melanoma | Surgery, | 8.16 | 199 | 41 | 16 |
| | | Pembro, Ipi | | | | |
| E | Urothelial | Surgery, Cis, | 0.88 | 18 | 1 | 13 |
| | | Gem | | | | |
| F | NSCLC | Surgery | 0.94 | 16 | 9 | 11 |
| G | Bladder | Surgery | 2.34 | 24 | 104 | 13 |
| H | Urothelial | Surgery, Cis, | 0.44 | 14 | 4 | 8 |
| | | Gem | | | | |
| K | SCCHN | Cetus, XRT | 3.19 | 15 | 15 | 9 |

As shown in Table 6 below, vaccination with GEN 009 resulted, after the priming series of three vaccinations at Day 1, Day 22 and Day 43, in detectable immune responses (i) in 100% of patients, and (ii) against 90% or more of individual patient-specific peptide antigens (SLPs corresponding to ATLAS-identified, patient-specific stimulatory antigens) across all patients and all FluoroSpot assays. Each number in Table 6 represents aggregate immunogenicity at Day 50 for all patient-specific peptide antigens (SLPs) for a given patient, by a given assay. The FluoroSpot assays are indicated as: ex vivo assay (complete PBMCs), ex vivo assay (enriched T cell subsets), and IVS assay (enriched T cell subsets). Both CD8+ and CD4+ T cell responses were observed. Ten-day in vitro stimulated FluoroSpot assays resulted in a greater proportion of positive and/or broader immune responses than the ex vivo FluoroSpot assays. In aggregate, all patient-specific peptide antigens (SLPs corresponding to ATLAS-identified, patient-specific stimulatory antigens) from the combined patients elicited:

Overall Response Rate: 99% of Peptides Positive

Effector T cell responses (for combined 8 patients A, B, C, E, F, G, H, and K), as detected by ex vivo assays (enriched T cell subsets):

CD4+=51%

CD8+=41%

Memory T cell responses (for combined 8 patients A, B, C, E, F, G, H, and K), as detected by IVS assays (enriched T cell subsets):

CD4+=87%

CD8+=59%
Total CD8+ T cell responses (for combined 8 patients A, B, C, E, F, G, H, and K), as detected by ex vivo and/or IVS assays (enriched T cell subsets)=74%
Total CD4+ T cell responses (for combined 8 patients A, B, C, E, F, G, H, and K), as detected by ex vivo and/or IVS assays (enriched T cell subsets)=92%
Total PBMC (combined CD4+ and CD8+ T cell) responses (for combined 8 patients A, B, C, E, F, G, H, and K), as detected by:
   Any assay: 91%
   ex vivo assays=45%
   IVS: 88%

Tables 6-7 show GEN 009 immunogenicity assays against patient-specific peptide antigens (SLPs), after priming series of three vaccinations at Day 1, Day 22 and Day 43.

TABLE 6

| | | ex vivo | | | | | | | |
|---|---|---|---|---|---|---|---|---|---|
| | Tumor | PBMC | | CD4 | | CD8 | | Total Pos | |
| Patient | Type | Baseline | D50* | Baseline | Day 50* | Baseline | Day 50* | Baseline | Day 50* |
| A | SqNSCLC | 0% | 80% | 0% | 10% | 10% | 20% | 10% | 80% |
| B | Urothelial | 100% | 75% | 88% | 50% | 63% | 50% | 100% | 75% |
| C | Melanoma | 19% | 63% | 19% | 6% | 19% | 38% | 44% | 81% |
| E | Urothelial | 0% | 31% | 0% | 100% | 8% | 69% | 8% | 100% |
| F | NSCLC | 45% | 45% | 55% | 55% | 0% | 45% | 55% | 82% |
| G | Urothelial | 8% | 15% | 8% | 77% | 23% | 15% | 31% | 85% |
| H | Bladder | 88% | 63% | 0% | 38% | 50% | 75% | 88% | 100% |
| K | SCHNCC | 11% | 22% | 11% | 89% | 11% | 11% | 33% | 89% |

TABLE 7

| | | IVS | | | | | | Any assay | |
|---|---|---|---|---|---|---|---|---|---|
| | | PBMC | | CD4 | | CD8 | | Total Pos | |
| Patient | Tumor Type | Baseline | Day 50* | Baseline | Day 50* | Baseline | Day 50* | Baseline | Day 50* |
| A | SqNSCLC | 0% | 100% | 10% | 67% | 0% | 33% | 10% | 100% |
| B | Urothelial | 0% | 75% | 75% | 100% | 50% | 63% | 63% | 100% |
| C | Melanoma | 100% | 100% | 100% | 100% | 69% | 100% | 100% | 100% |
| E | Urothelial | 8% | 69% | 46% | 85% | 0% | 31% | 54% | 85% |
| F | NSCLC | 27% | 82% | 18% | 100% | 55% | 64% | 64% | 100% |
| G | Urothelial | 62% | 100% | 92% | 77% | 23% | 62% | 92% | 100% |
| H | Bladder | 100% | 88% | 50% | 8% | 63% | 63% | 100% | 88% |
| K | SCHNCC | 11% | 78% | 0% | 78% | 11% | 33% | 22% | 78% |

Data are presented as the proportion of peptides defined as positive by the DFR(eq) test (p<0.05) at the indicated time point for each cell type.
*“Day 50” indicates 50 days post initial vaccination.

FIG. 2 shows representative results of in vitro stimulated FluoroSpot assays on CD4+ and CD8+ T cells enriched from PBMCs collected at baseline (prior to vaccination) and at Day 50 from each of 5 patients (patients A, B, C, E, and F). Data are represented as the mean IFN-γ spot forming cells (SFC)+/−SEM per 10,000 or 20,000 T cells, as indicated, for a given patient, for each of the 4 pools of SLPs (each pool comprising 1-5 SLPs) included in that patient's vaccine.

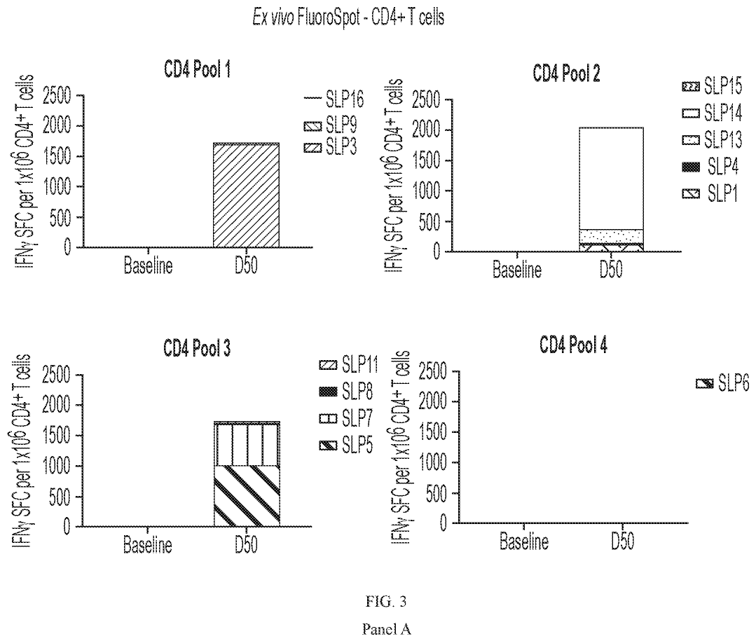

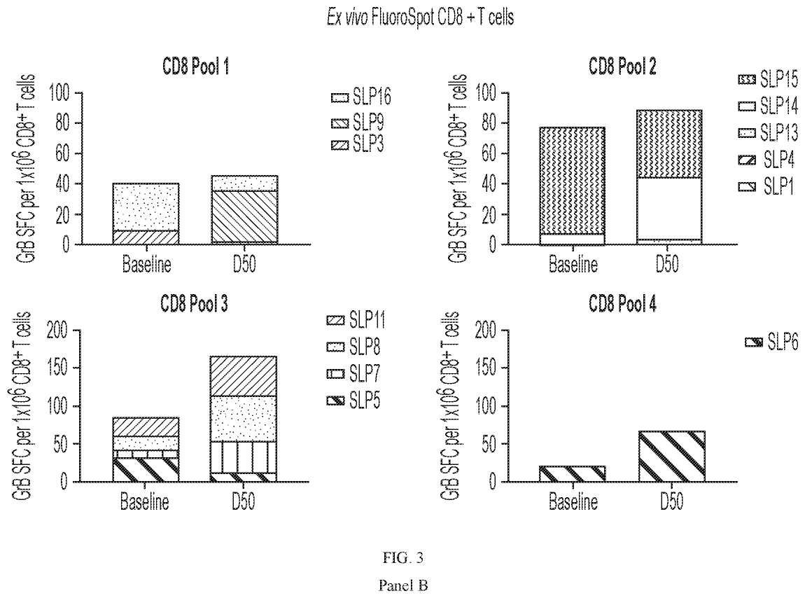

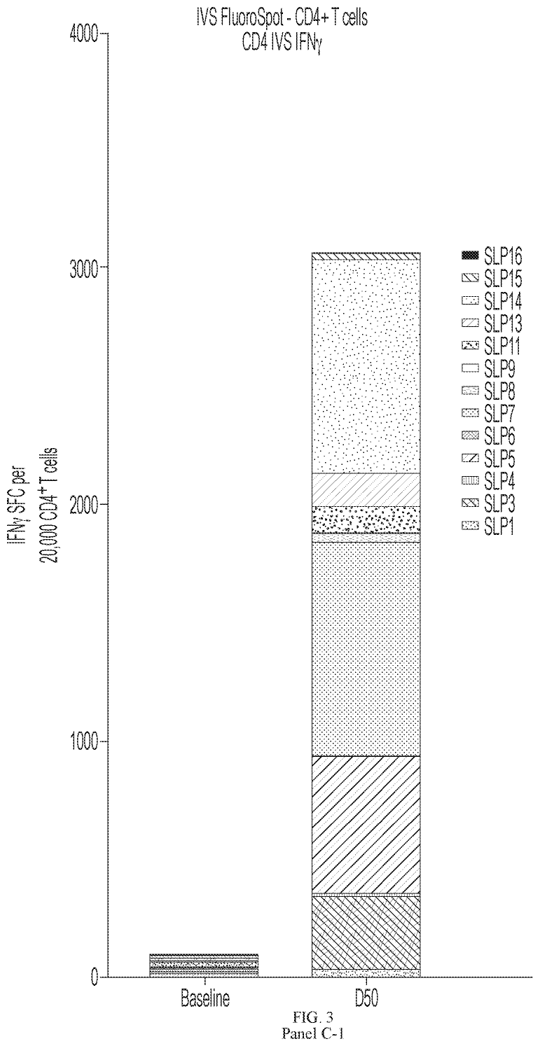

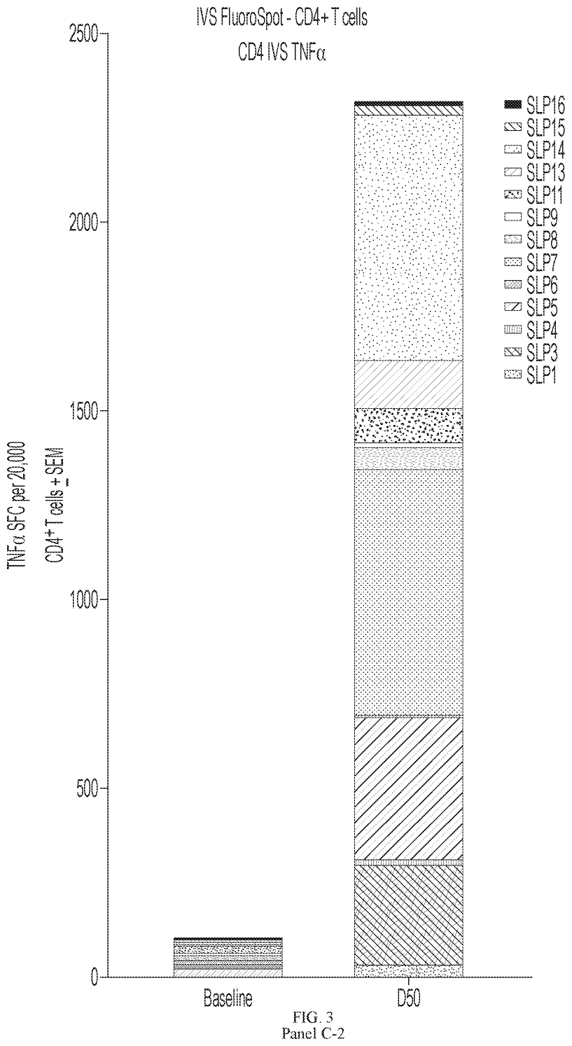

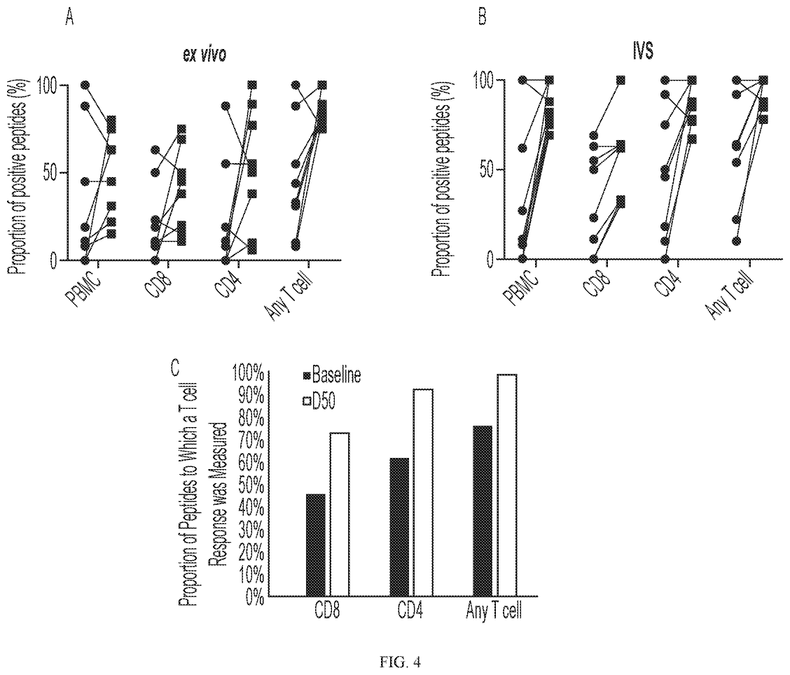

FIG. 3 shows representative results of ex vivo FluoroSpot assays and in vitro stimulated FluoroSpot assays on CD4+ and CD8+ T cells enriched from PBMCs collected at baseline (prior to vaccination) and at Day 50 from a representative patient (patient E). Panels A and B: ex vivo FluoroSpot assays. Data are represented as the mean cytokine spot forming cells (SFC) per million T cells, for each SLP included in the patient's vaccine, as indicated. Panel C: in vitro stimulated FluoroSpot assays. Data are represented as the mean cytokine spot forming cells (SFC) per 20,000 T cells, for each SLP included in the patient's vaccine, as indicated.

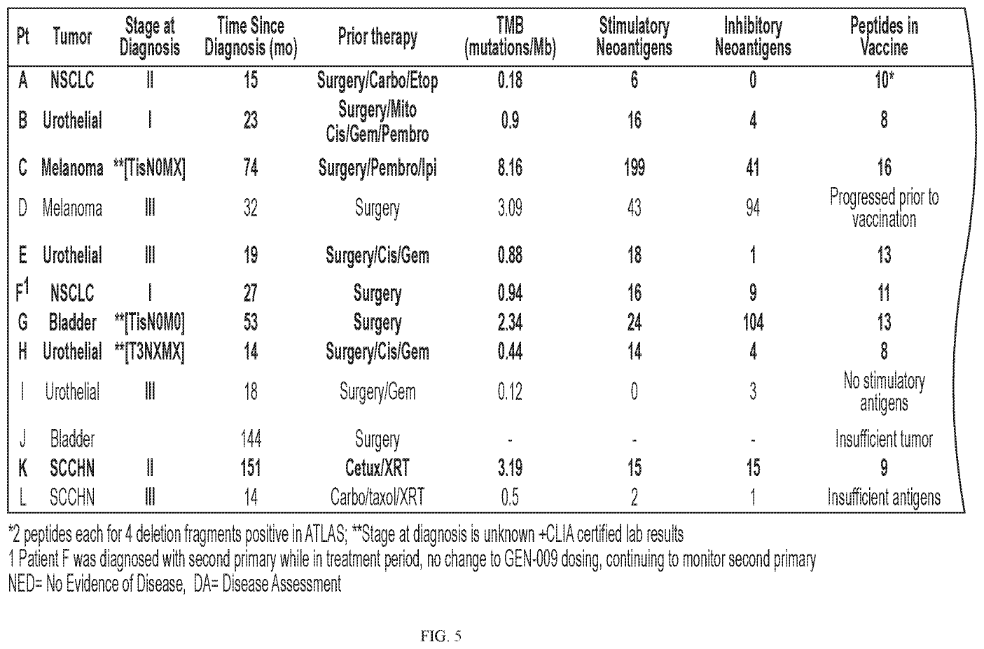

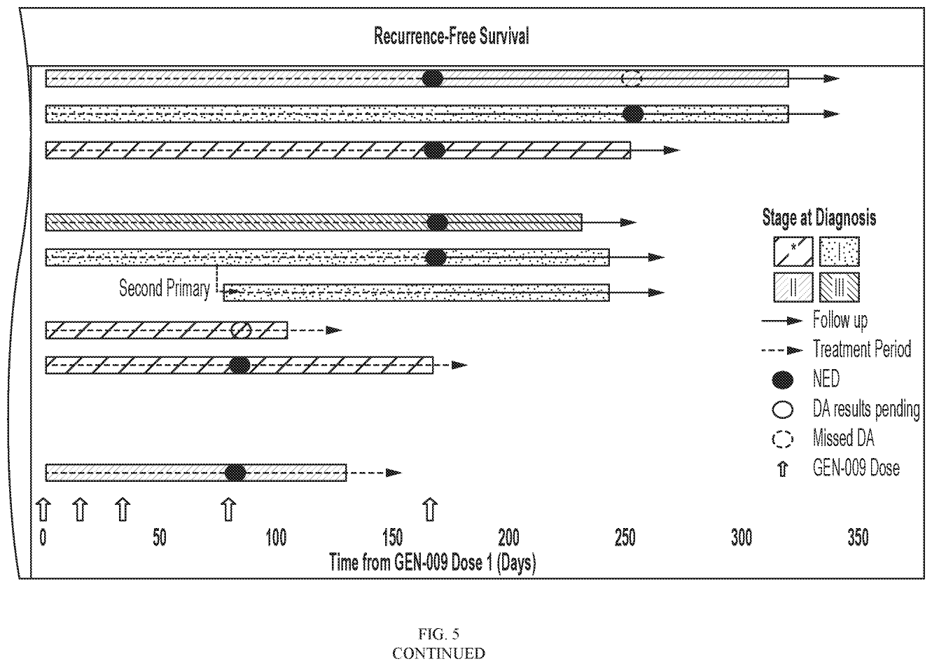

FIG. 4 shows representative summary results of ex vivo FluoroSpot assays and in vitro stimulated FluoroSpot assays on total PBMC or PBMCs depleted of CD4+ or CD8+ T cells collected at baseline (prior to vaccination) and at Day 50 from patients A-H and K. Data are reported as the proportion of peptides positive by the DFR(eq) test. Circles represent baseline, squares represent D50 time point. For results shown in Panel A, 200,000 total PBMC or PBMCs depleted of CD4+ or CD8+ T cells were stimulated with overlapping peptides (OLPs) spanning each immunized SLP in an IFNγ and Granzyme B (GrB) dual color ex vivo fluorospot assay. For results as shown in Panel B, PBMCs depleted of CD4+ or CD8+ T cells were stimulated with OLPs for 10 days followed by an overnight IFNγ and TNFα dual color fluorospot assay. Panel C shows the proportion of SLPs scored positive by any assay for patients A-H, and K.

Data from patients A-L are shown in FIG. 5, including for each patient, the tumor type, stage of cancer at diagnosis, period of time from diagnosis, prior therapies the patient received, the patients calculated tumor mutational burden (TMB), the number of stimulatory and inhibitory neoantigens identified for each patient, and the number of peptides in the vaccine administered. The graph indicates the status of each patient at different points within the example vaccination regimen. The timing of vaccination is indicated by a vertical arrows. The color of the horizontal bars indicate the stage of cancer at diagnosis. A blue horizontal arrow indicates that the patient has not yet completed the vaccination regimen (i.e., is within the dosign period). A black horizontal arrow indicates that the patient has completed the vaccination regimen (i.e., is past the treatment period or post vaccination schedule). A black circle indicates a status of “NED” or no evidence of disease. The graph shows that all patients post vaccination experienced recurrence-free survival for at least 4 months.

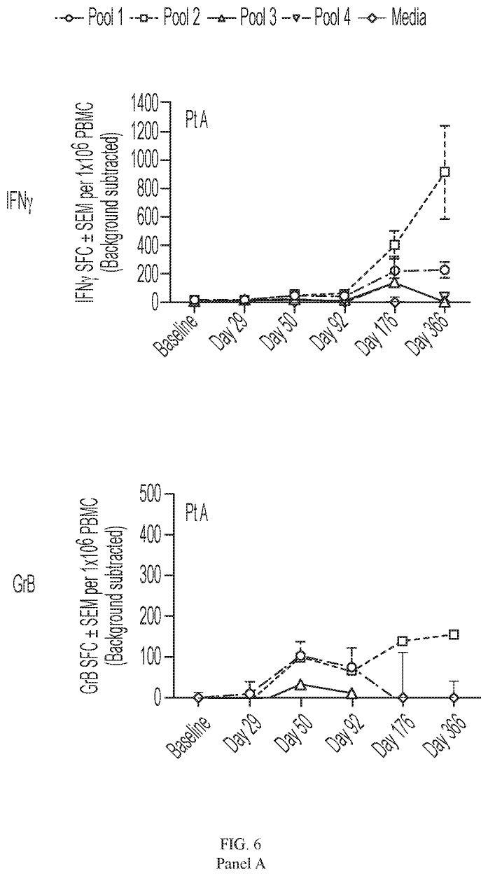

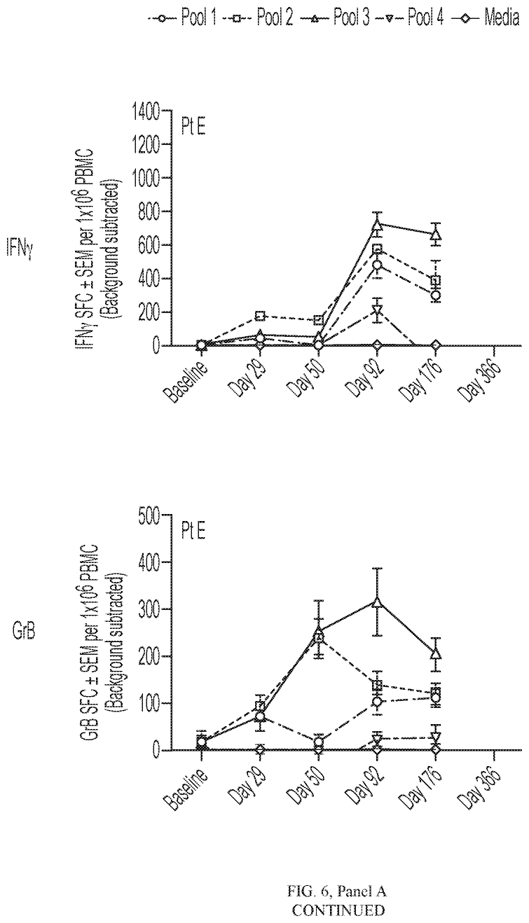

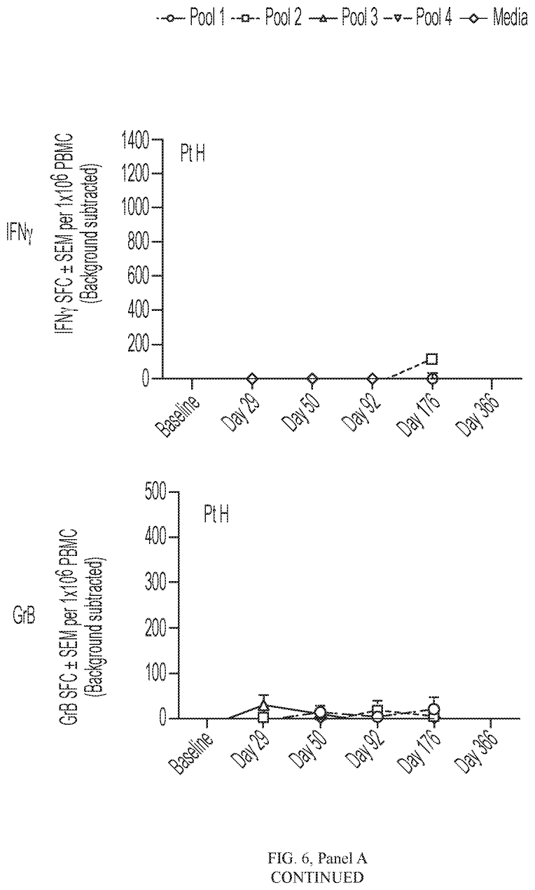

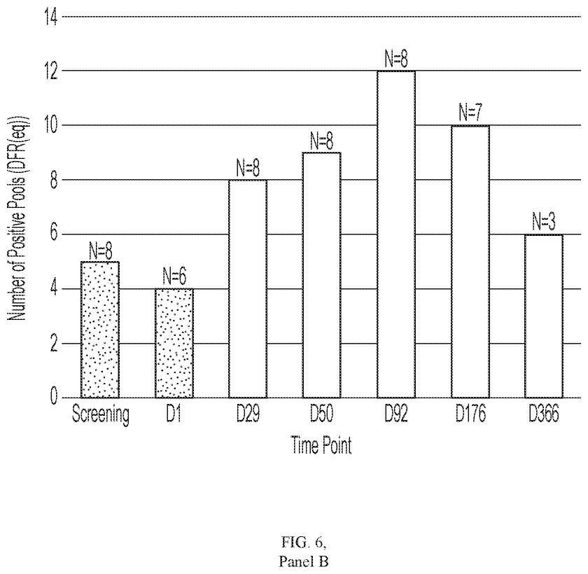

In subsequent follow-up, 7 of 8 vaccinated patients had no disease progression after median follow-up of 14 months (range 8 to 17 months). This outcome compares favorably to the expected relapse rates in these malignancies. The patient who progressed (H) had low immune responses (see FIG. 6, Panel A), but exceeded previous remissions.

FIG. 6 shows representative results of ex vivo dual-analyte FluoroSpot assays on CD4+ and CD8+ T cells enriched from PBMCs of three representative patients (patients A and E; low response patient H). Bulk PBMCs were isolated from the patients at baseline (prior to vaccination) and at the indicated timepoints over the course of their treatment. The secretion of IFNγ and Granzyme B (GrB) was quantified via ex vivo dual-analyte FluoroSpot after stimulation with overlapping peptide pools (OLPs) spanning the patient-specific SLPs used for immunization. In Panel A, data are expressed as mean (±SEM) spot forming cells (SFC) per million PBMCs to each of the four pools. Panel B shows the number of positive pools for each time point.

These results demonstrate that immune responses developed early and were seen at the first sampling post initial vaccination at day 29, and were also found as far out as 12 months, 6 months after completion of vaccination. Peak ex vivo T cell responses occurred at 3 months, after 3 vaccinations.

### REFERENCES

1. Hacohen N, Fritsch E F, Carter T A, Lander E S, Wu C J. Getting personal with neoantigen-based therapeutic cancer vaccines. Cancer Immunol Res. 2013; 1(1):11-5.
2. Heemskerk B, Kvistborg P, Schumacher T N. The cancer antigenome. EMBO J. 2013; 32(2):194-203.
3. Castle J C, Kreiter S, Diekmann J, Lower M, van de Roemer N, de Graaf J, et al. Exploiting the mutanome for tumor vaccination. Cancer Res. 2012; 72(5):1081-91.
4. Lawrence M S, Stojanov P, Polak P, Kryukov G V, Cibulskis K, Sivachenko A, et al. Mutational heterogeneity in cancer and the search for new cancer-associated genes. Nature. 2013; 499(7457):214-8
5. Berd D, Murphy G, Maguire H C, Jr., Mastrangelo M J. Immunization with haptenized, autologous tumor cells induces inflammation of human melanoma metastases. Cancer Res. 1991; 51(10):2731-4.
6. Randazzo M, Terness P, Opelz G, Kleist C. Active-specific immunotherapy of human cancers with the heat shock protein Gp96-revisited. Int J Cancer. 2012; 130(10):2219-31.
7. Senzer N N, Kaufman H L, Amatruda T, Nemunaitis M, Reid T, Daniels G, et al. Phase II clinical trial of a granulocyte-macrophage colony-stimulating factor-encoding, second-generation oncolytic herpesvirus in patients with unresectable metastatic melanoma. J Clin Oncol. 2009; 27(34):5763-71.
8. Kvistborg P, Philips D, Kelderman S, Hageman L, Ottensmeier C, Joseph-Pietras D, et al. Anti-CTLA-4 therapy broadens the melanoma-reactive CD8+ T cell response. Sci Transl Med. 2014; 6(254):254ra128.
9. Bystryn J C, Zeleniuch-Jacquotte A, Oratz R, Shapiro R L, Harris M N, Roses D F. Double-blind trial of a polyvalent, shed-antigen, melanoma vaccine. Clin Cancer Res. 2001; 7(7):1882-7.
10. Lennerz V, Fatho M, Gentilini C, Frye R A, Lifke A, Ferel D, et al. The response of autologous T cells to a human melanoma is dominated by mutated neoantigens. Proc Natl Acad Sci USA. 2005; 102(44):16013-8.
11. Zhou J, Dudley M E, Rosenberg S A, Robbins P F. Persistence of multiple tumor-specific T-cell clones is associated with complete tumor regression in a melanoma patient receiving adoptive cell transfer therapy. J Immunother. 2005; 28(1):53-62.
12. Tran E, Turcotte S, Gros A, Robbins P F, Lu Y C, Dudley M E, et al. Cancer immunotherapy based on mutation-specific CD4+ T cells in a patient with epithelial cancer. Science. 2014; 344(6184):641-5.
13. Abelin J G, Keskin D B, Sarkizova S, Hartigan C R, Zhang W, Sidney J, et al. Mass Spectrometry Profiling of HLA-Associated Peptidomes in Mono-allelic Cells Enables More Accurate Epitope Prediction. Immunity. 2017; 46(2):315-26.
14. Slingluff C L, Jr. The present and future of peptide vaccines for cancer: single or multiple, long or short, alone or in combination? Cancer J. 2011; 17(5):343-50.
15. Ammi R, De Waele J, Willemen Y, Van Brussel I, Schrijvers D M, Lion E, et al. Poly(I:C) as cancer vaccine adjuvant: knocking on the door of medical breakthroughs. Pharmacol Ther. 2015; 146:120-31.
16. Okada H, Butterfield L H, Hamilton R L, Hoji A, Sakaki M, Ahn B J, et al. Induction of robust type-1 CD8+ T-cell responses in WHO grade 2 low-grade glioma patients receiving peptide-based vaccines in combination with poly-ICLC. Clin Cancer Res. 2015; 21(2):286-94.
17. Rapoport A P, Aqui N A, Stadtmauer E A, Vogl D T, Xu Y Y, Kalos M, et al. Combination immunotherapy after ASCT for multiple myeloma using MAGE-A3/Poly-ICLC immunizations followed by adoptive transfer of vaccine-primed and costimulated autologous T cells. Clin Cancer Res. 2014; 20(5):1355-65.
18. Sabbatini P, Tsuji T, Ferran L, Ritter E, Sedrak C, Tuballes K, et al. Phase I trial of overlapping long peptides from a tumor self-antigen and poly-ICLC shows rapid induction of integrated immune response in ovarian cancer patients. Clin Cancer Res. 2012; 18(23):6497-508.
19. Sharma S, Zhu L, Davoodi M, Harris-White M, Lee J M, St John M, et al. TLR3 agonists and proinflammatory antitumor activities. Expert Opin Ther Targets. 2013; 17(5):481-3.
20. Kimura T, McKolanis J R, Dzubinski L A, Islam K, Potter D M, Salazar A M, et al. MUC1 vaccine for individuals with advanced adenoma of the colon: a cancer immunoprevention feasibility study. Cancer Prev Res (Phila). 2013; 6(1):18-26.
21. Kobayashi Y, Sakura T, Miyawaki S, Toga K, Sogo S, Heike Y. A new peptide vaccine OCV-501: in vitro pharmacology and phase 1 study in patients with acute myeloid leukemia. Cancer Immunol Immunother. 2017; 66(7):851-63.
22. Obara W, Sato F, Takeda K, Kato R, Kato Y, Kanehira M, et al. Phase I clinical trial of cell division associated 1 (CDCA1) peptide vaccination for castration resistant prostate cancer. Cancer Sci. 2017; 108(7):1452-7.
23. Suekane S, Ueda K, Nishihara K, Sasada T, Yamashita T, Koga N, et al. Personalized peptide vaccination as second-line treatment for metastatic upper tract urothelial carcinoma. Cancer Sci. 2017; 108(12):2430-7.
24. Ott P A, Hu Z, Keskin D B, Shukla S A, Sun J, Bozym D J, et al. An immunogenic personal neoantigen vaccine for patients with melanoma. Nature. 2017; 547(7662):217-21.

### EQUIVALENTS

It is to be understood that while the disclosure has been described in conjunction with the detailed description thereof, the foregoing description is intended to illustrate and not limit the scope of the invention, which is defined by the scope of the appended claims. Other aspects, advantages, and modifications are within the scope of the following claims:

We claim:

1. A method of inducing an immune response in a subject, the method comprising administering to the subject an immunogenic composition comprising one or more selected stimulatory antigens (e.g., one or more stimulatory antigens described herein) or immunogenic fragments thereof, wherein the immunogenic composition is administered according to a dosing regimen comprising an initial dose of the immunogenic composition and additional doses of the immunogenic composition, wherein after an initial dose is administered, an additional dose is administered 3 weeks following the initial dose, an additional dose is administered 6 weeks following the initial dose, an additional dose is administered 12 weeks following the initial dose, and an additional dose is administered 24 weeks following the initial dose.

2. The method of claim 1, wherein the immunogenic composition comprises one or more stimulatory antigens selected by:
a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject;
b) contacting the bacterial cells or beads with antigen presenting cells (APCs) from the subject, wherein the APCs internalize the bacterial cells or beads;
c) contacting the APCs with lymphocytes from the subject, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer.

3. The method of claim 1, further comprising:
a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject;
b) contacting the bacterial cells or beads with antigen presenting cells (APCs) from the subject, wherein the APCs internalize the bacterial cells or beads;
c) contacting the APCs with lymphocytes from the subject, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer.

4. The method of claim 1, wherein the immunogenic composition does not comprise a selected inhibitory antigen (e.g., an inhibitory antigen described herein).

5. The method of claim 1, wherein the immunogenic composition does not comprise an inhibitory antigen selected by:
a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject;
b) contacting the bacterial cells or beads with antigen presenting cells (APCs) from the subject, wherein the APCs internalize the bacterial cells or beads;
c) contacting the APCs with lymphocytes from the subject, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more inhibitory antigens, from among the identified tumor antigens (i) one or more tumor antigens that increase level of expression and/or secretion of one or more immune mediators associated with at least one deleterious and/or non-beneficial response to cancer, and/or (ii) one or more tumor antigens that inhibit and/or suppress level of expression and/or secretion of one or more immune mediators associated with at least one beneficial response to cancer.

6. The method of claim 4, further comprising:
a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject;
b) contacting the bacterial cells or beads with antigen presenting cells (APCs) from the subject, wherein the APCs internalize the bacterial cells or beads;
c) contacting the APCs with lymphocytes from the subject, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen; and
f) selecting as one or more inhibitory antigens, from among the identified tumor antigens (i) one or more tumor antigens that increase level of expression and/or secretion of one or more immune mediators associated with at least one deleterious and/or non-beneficial response to cancer, and/or (ii) one or more tumor antigens that inhibit and/or suppress level of expression and/or secretion of one or more immune mediators associated with at least one beneficial response to cancer.

7. A method of inducing an immune response in a subject, the method comprising:
a) obtaining, providing, or generating a library comprising bacterial cells or beads, wherein each bacterial cell or bead of the library comprises a different heterologous polypeptide comprising one or more mutations, splice variants, or translocations expressed in a cancer or tumor cell of a subject;
b) contacting the bacterial cells or beads with antigen presenting cells (APCs) from the subject, wherein the APCs internalize the bacterial cells or beads;
c) contacting the APCs with lymphocytes from the subject, under conditions suitable for activation of lymphocytes by a polypeptide presented by one or more APCs;
d) determining whether one or more lymphocytes are activated by, or not responsive to, one or more polypeptides presented by one or more APCs, e.g., by assessing (e.g., detecting or measuring) a level (e.g., an increased or decreased level, relative to a control), of expression and/or secretion of one or more immune mediators;
e) identifying one or more polypeptides that stimulate, inhibit and/or suppress, and/or have a minimal effect on level of expression and/or secretion of one or more immune mediators, wherein stimulation, inhibition and/or suppression indicate that the polypeptide is a tumor antigen;
f) selecting as one or more stimulatory antigens, from among the identified tumor antigens (i) one or more tumor antigens that have a minimal effect on level of expression and/or secretion of one or more immune mediators, (ii) one or more tumor antigens that increase level of expression and/or secretion of one or more immune mediators associated with at least one beneficial response to cancer; and/or (iii) one or more tumor antigens that inhibit and/or suppress level of expression and/or secretion of one or more immune mediators associated with at least one deleterious and/or non-beneficial response to cancer; and
g) administering to the subject multiple doses of an immunogenic composition comprising one or more of the selected stimulatory antigens, or immunogenic fragments thereof, wherein after an initial dose is administered, a dose is administered 3 weeks following the initial dose, a dose is administered 6 weeks following the initial dose, a dose is administered 12 weeks following the initial dose, and a dose is administered 24 weeks following the initial dose.

8. The method of claim 7, wherein the immunogenic composition does not comprise a selected inhibitory antigen (e.g., an inhibitory antigen described herein).

9. The method of claim 8, wherein one or more of the identified tumor antigens is selected as an inhibitory antigen if (i) the one or more tumor antigens increase level of expression and/or secretion of one or more immune mediators associated with at least one deleterious and/or non-beneficial response to cancer, and/or (ii) the one or more tumor antigens inhibit and/or suppress level of expression and/or secretion of one or more immune mediators associated with at least one beneficial response to cancer.

10. The method of claim 8, further comprising selecting as one or more inhibitory antigens, from among the identified tumor antigens (i) one or more tumor antigens that increase level of expression and/or secretion of one or more immune mediators associated with at least one deleterious and/or non-beneficial response to cancer, and/or (ii) one or more tumor antigens that inhibit and/or suppress level of expression and/or secretion of one or more immune mediators associated with at least one beneficial response to cancer.

11. The method of any one of claims 2-10, wherein the library comprises bacterial cells or beads comprising at least 1, 3, 5, 10, 15, 20, 25, 30, 50, 100, 150, 250, 500, 750, 1000 or more different heterologous polypeptides, or portions thereof.

12. The method of any one of claims 2-11, wherein determining whether one or more lymphocytes are activated by, or not responsive to, one or more tumor antigens comprises measuring a level of one or more immune mediators.

13. The method of any one of claims 2-12, wherein the one or more immune mediators are selected from the group consisting of cytokines, soluble mediators, and cell surface markers expressed by the lymphocytes.

14. The method of any one of claims 12-13, wherein the one or more immune mediators are cytokines.

15. The method of claim 14, wherein the one or more cytokines are selected from the group consisting of TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANK L), MIP3-alpha, and fractalkine.

16. The method of any one of claims 2-15, wherein the one or more immune mediators are soluble mediators.

17. The method of claim 16, wherein the one or more soluble mediators are selected from the group consisting of granzyme A, granzyme B, sFas, sFasL, perforin, and granulysin.

18. The method of any one of claims 2-17, wherein the one or more immune mediators are cell surface markers.

19. The method of claim 18, wherein the one or more cell surface markers are selected from the group consisting of CD107a, CD107b, CD25, CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), and KLRG1.

20. The method of any one of claims 2-19, wherein the lymphocytes comprise CD4+ T cells.

21. The method of any one of claims 2-19, wherein the lymphocytes comprise CD8+ T cells.

22. The method of any one of claims 2-19, wherein the lymphocytes comprise NKT cells, gamma-delta T cells, or NK cells.

23. The method of any one of claims 2-19, wherein the lymphocytes comprise any combination of CD4+ T cells, CD8+ T cells, NKT cells, gamma-delta T cells, and NK cells.

24. The method of any one of claims 2-23, wherein lymphocyte activation is determined by assessing a level of one or more expressed or secreted immune mediators that is at least 20%, 40%, 60%, 80%, 100%, 120%, 140%, 160%, 180%, or 200% higher or lower than a control level.

25. The method of any one of claims 2-23, wherein lymphocyte activation is determined by assessing a level of one or more expressed or secreted immune mediators that is at least one, two, or three standard deviations greater or lower than the mean of a control level.

26. The method of any one of claims 2-23, wherein lymphocyte activating is determined by assessing a level of one or more expressed or secreted immune mediators that is at least 1, 2, 3, 4 or 5 median absolute deviations (MADs) greater or lower than a median response level to a control.

27. The method of any one of claims 2-23, wherein lymphocyte non-responsiveness is determined by assessing a level of one or more expressed or secreted immune mediators that is within 5%, 10%, 15%, or 20% of a control level.

28. The method of any one of claims 2-23, wherein lymphocyte non-responsiveness is determined by assessing a level of one or more expressed or secreted immune mediators that is less than one or two standard deviation higher or lower than the mean of a control level.

29. The method of any one of claims 2-23, wherein lymphocyte non-responsiveness is determined by assessing a level of one or more expressed or secreted immune mediators that is less than one or two median absolute deviation (MAD) higher or lower than a median response level to a control.

30. The method of any one of claims 1-29, wherein a subject exhibits at least one measure or indication of clinical responsiveness to a cancer therapy.

31. The method of any one of claims 1-29, wherein a subject exhibits at least one measure or indication of failure of clinical responsiveness to a cancer therapy.

32. The method of claim 30 or 31, wherein the cancer therapy comprises immune checkpoint blockade therapy.

33. The method of claim 32, wherein the immune checkpoint blockade therapy comprises administration of pembrolizumab, nivolumab, ipilimumab, atezolizumab, avelumab, durvalumab, tremelimumab, or cemiplimab.

34. The method of claim 32 or 33, wherein the immune checkpoint blockade therapy comprises administration of two or more immune checkpoint inhibitors.

35. The method of claim 30 or 31, wherein the cancer therapy comprises immune suppression blockade therapy.

36. The method of claim 35, wherein the immune suppression blockade therapy comprises administration of Vista (B7-H5, v-domain Ig suppressor of T cell activation) inhibitors, Lag-3 (lymphocyte-activation gene 3, CD223) inhibitors, IDO (indolemamine-pyrrole-2,3,-dioxygenase-1,2) inhibitors, or KIR receptor family (killer cell immunoglobulin-like receptor) inhibitors, CD47 inhibitors, or Tigit (T cell immunoreceptor with Ig and ITIM domain) inhibitors.

37. The method of claim 35 or 36, wherein the immune suppression blockade therapy comprises administration of two or more immune suppression inhibitors.

38. The method of claim 30 or 31, wherein the cancer therapy comprises immune activation therapy.

39. The method of claim 38, wherein the immune activation therapy comprises administration of CD40 agonists, GITR (glucocorticoid-induced TNF-R-related protein, CD357) agonists, OX40 (CD134) agonists, 4-1BB (CD137) agonists, ICOS (inducible T cell stimulator, CD278) agonists, IL-2 (interleukin 2) agonists, or interferon agonists.

40. The method of claim 38 or 39, wherein the immune activation therapy comprises administration of two or more immune activators.

41. The method of claim 30 or 31, wherein the cancer therapy comprises adjuvant therapy.

42. The method of claim 41, where the adjuvant therapy comprises administration of a TLR agonist (e.g., CpG or Poly J:C), STING agonist, non-specific stimulus of innate immunity, dendritic cells, GM-CSF, IL-12, IL-7, Flt-3, or other cytokines.

43. The method of claim 30 or 31, wherein the cancer therapy comprises oncolytic virus therapy.

44. The method of claim 43, wherein the oncolytic viral therapy comprises administration of talimogene leherparepvec.

45. The method of claim 30 or 31, wherein the cancer therapy comprises administration of one or more chemotherapeutic agents.

46. The method of claim 30 or 31, wherein the cancer therapy comprises radiation.

47. The method of claim 30 or 31, wherein the cancer therapy comprises surgical excision.

48. The method of claim 30 or 31, wherein the cancer therapy comprises cell-based therapy.

49. The method of claim 48, wherein the cell-based therapy comprises administration of dendritic cells, chimeric antigen receptor T (CAR-T) cells, T cell receptor-transduced cells, tumor infiltrating lymphocytes (TIL), or natural killer (NK) cells.

50. The method of claim 30 or 31, wherein the cancer therapy comprises localized hyperthermia or hypothermia.

51. The method of claim 30 or 31, wherein the cancer therapy comprises administration of one or more anti-tumor antibodies.

52. The method of claim 51, wherein the anti-tumor antibodies comprise bi-specific antibodies.

53. The method of claim 30 or 31, wherein the cancer therapy comprises administration of one or more anti-angiogenic agents.

54. The method of claim 30 or 31, wherein the cancer therapy comprises any combination of immune checkpoint blockade, immune suppression blockade, immune activation, adjuvant, oncolytic virus, chemotherapeutic, radiation, surgical, cell-based, hyperthermia, hypothermia, anti-tumor antibody, and anti-angiogenic therapies.

55. The method of any one of claims 1-54, wherein the subject has or is at risk of cancer, and/or exhibits one or more signs or symptoms of cancer, and/or exhibits one or more risk factors for cancer.

56. The method of claim 55, wherein the cancer is colorectal cancer, melanoma, bladder cancer, or lung cancer (e.g., non-small cell lung cancer).

57. The method of any one of claims 1-56, wherein the immune response comprises activation of one or more lymphocytes.

58. The method of claim 57, wherein the one or more lymphocytes comprise CD4+ T cells.

59. The method of claim 57 or 58, wherein the one or more lymphocytes comprise CD8+ T cells.

60. The method of any one of claims 57-59, wherein the one or more lymphocytes comprise NKT cells, gamma-delta T cells, or NK cells.

61. The method of any one of claims 57-60, wherein the one or more lymphocytes comprise any combination of CD4+ T cells, CD8+ T cells, NKT cells, gamma-delta T cells, and NK cells.

62. The method of any one of claims 1-61, wherein the immune response comprises an increased expression and/or secretion of one or more immune mediators relative to a control.

63. The method of claim 62, wherein the one or more immune mediators are cytokines.

64. The method of claim 63, wherein the cytokines are selected from TRAIL, IFN-gamma, IL-12p70, IL-2, TNF-alpha, MIP1-alpha, MIP1-beta, CXCL9, CXCL10, MCP1, RANTES, IL-1 beta, IL-4, IL-6, IL-8, IL-9, IL-10, IL-13, IL-15, CXCL11, IL-3, IL-5, IL-17, IL-18, IL-21, IL-22, IL-23A, IL-24, IL-27, IL-31, IL-32, TGF-beta, CSF, GM-CSF, TRANCE (also known as RANK L), MIP3-alpha, MCP1, and fractalkine.

65. The method of claim 62, wherein the one or more immune mediators are soluble mediators.

66. The method of claim 65, wherein the one or more soluble mediators are selected from granzyme A, granzyme B, sFas, sFasL, perform, and granulysin.

67. The method of claim 62, wherein the one or more immune mediators are cell surface markers.

68. The method of claim 67, wherein the cell surface markers are selected from CD107a, CD107b, CD25, CD69, CD45RA, CD45RO, CD137 (4-1BB), CD44, CD62L, CD27, CCR7, CD154 (CD40L), KLRG-1, CD71, HLA-DR, CD122 (IL-2RB), CD28, IL7Ra (CD127), CD38, CD26, CD134 (OX-40), CTLA-4 (CD152), LAG-3, TIM-3 (CD366), CD39, PD1 (CD279), FoxP3, TIGIT, CD160, BTLA, 2B4 (CD244), and KLRG1.

69. The method of any one of claims 62-68, wherein a level of one or more expressed or secreted immune mediators that is at least 20%, 40%, 60%, 80%, 100%, 120%, 140%, 160%, 180%, or 200% higher than a control level indicates lymphocyte activation.

70. The method of any one of claims 62-68, wherein a level of one or more expressed or secreted immune mediators that is at least one, two, or three standard deviations higher than the mean of a control level indicates lymphocyte activation.

71. The method of any one of claims 62-68, wherein a level of one or more expressed or secreted immune mediators that is at least 1, 2, 3, 4 or 5 median absolute deviations (MADs) higher or lower than a median response level to a control indicates lymphocyte activation.

72. The method of any one of claims 1-71, wherein the immune response comprises a humoral response and/or a cellular response.

73. The method of claim 72, wherein the humoral response comprises an increase in magnitude of response or fold rise from baseline of antigen specific immunoglobulin G (IgG) levels and/or of antigen specific neutralizing antibody levels.

74. The method of claim 72 or 73, wherein the humoral response comprises a 4-fold or greater rise in IgG titer from baseline.

75. The method of any one of claims 72-74, wherein the humoral response comprises a 2-fold or greater rise in 50% neutralizing antibody titer from baseline.

76. The method of any one of claims 72-75, wherein the cellular response comprises secretion of granzyme B (GrB).

77. The method of any one of claims 72-76, wherein the cellular response comprises an increase in magnitude of response or fold rise from baseline of granzyme B (GrB) levels.

78. The method of any one of claims 72-77, wherein the cellular response comprises an increase in IFN-gamma secretion for T cells.

79. The method of any one of claims 1-78, wherein the selected stimulatory antigens comprise (i) a tumor antigen described herein (e.g., comprising an amino acid sequence described herein), (ii) a polypeptide having an amino acid sequence at least 90%, 91%, 92%, 93%, 94%, 95%, 96%, 97%, 98%, 99% identical to the amino acid sequence of a tumor antigen described herein, and/or (iii) a polypeptide comprising the amino acid sequence of a tumor antigen described herein having at least one deletion, insertion, and/or translocation.

80. The method of any one of claims 1-79, wherein the immunogenic composition comprises an adjuvant.

81. The method of claim 80, wherein the adjuvant comprises poly-ICLC.

82. The method of any one of claims 1-81, wherein the immunogenic composition comprises synthetic stimulatory antigens.

83. The method of claim 82, wherein the synthetic stimulatory antigens are synthetic long peptides (SLPs).

84. The method of claim 82 or 83, wherein the immunogenic composition comprises 1, 2, 3, 4, 5, 6, 7, 8, 9, or 10 SLPs.

85. The method of claim 83 or 84, comprising administering to the subject 2, 3, 4, 5, 6, 7, or 8 immunogenic compositions comprising SLPs.

86. The method of any one of claims 83-85, comprising administering to the subject 4 different immunogenic compositions, each immunogenic composition comprising 1 to 5 different SLPs.

87. The method of any one of claims 83-86, wherein each immunogenic composition comprises about 100 to about 1500 μg total peptide.

88. The method of any one of claims 1-87, further comprising administering to the subject a cancer therapy or combination of therapies.
